(19) United States
(12) Patent Application Publication
(10) Pub. No.: US 2020/0390717 A1
(43) Pub. Date: Dec. 17, 2020

(54) SYNTHETICALLY LETHAL NANOPARTICLES FOR TREATMENT OF CANCERS

(71) Applicant: UNIVERSITY OF IOWA RESEARCH FOUNDATION, Iowa City, IA (US)

(72) Inventors: Aliasger K. Salem, Coralville, IA (US); Kristina Thiel, Iowa City, IA (US); Kimberly Leslie, Iowa City, IA (US); Kareem Ebeid, Coralville, IA (US)

(21) Appl. No.: 16/766,216

(22) PCT Filed: Nov. 20, 2018
(86) PCT No.: PCT/US18/62025

§ 371 Date: May 21, 2020

Related U.S. Application Data
(60) Provisional application No. 62/589,288, filed on Nov. 21, 2017.

(51) Int. Cl. A61K 9/51; A61K 31/337; A61K 31/496; A61K 47/22; A61K 47/10

(52) U.S. Cl. CPC A61K 9/5153; A61K 31/337; B82Y 5/00; A61K 47/22; A61K 47/10; A61K 31/496

(57) ABSTRACT

Disclosed are nanoparticle compositions and methods for treating cancer in a subject in need thereof. The nanoparticle compositions and methods may be utilized to treat cancers in a subject that are characterized by susceptibility to synthetic lethality via administering a combination of agents that induce synthetic lethality.

### CROSS-REFERENCE TO RELATED PATENT APPLICATIONS

The present application claims the benefit of priority under 35 U.S.C. § 119(e) to U.S. Provisional Application No. 62/589,288, the content of which is incorporated herein by reference in its entirety.

### BACKGROUND

The field of the invention relates to nanoparticle compositions and their methods of use for treating cancer in a subject. The field of the invention also relates to nanoparticle compositions and their methods for treating cancers in a subject that are characterized by susceptibility to synthetic lethality via administering a combination of agents that induce synthetic lethality, such as uterine cancers and breast cancers characterized by loss-of-function of the p53 protein and/or the breast cancer 1 (BRCA1) protein.

In particular, uterine serous carcinoma (USC) is one of the most aggressive types of endometrial cancer and is characterized by poor outcomes and mutations in the tumor suppressor p53. Here, the inventors achieved synthetic lethality to paclitaxel (PTX), the frontline treatment for uterine serous carcinoma, in tumors with mutant p53 and enhanced therapeutic efficacy using polymeric nanoparticles. The inventors also identified the optimal nanoparticle formulation through a comprehensive analysis of release profiles, cellular uptake and cell viability.

### SUMMARY

Disclosed are nanoparticle compositions and methods for treating cancer in a subject in need thereof. The nanoparticle compositions and methods may be utilized to treat cancers in a subject that are characterized by susceptibility to synthetic lethality via administering a combination of agents that induce synthetic lethality

In some embodiments, the nanoparticle compositions and methods may be utilized to treat cancers that are characterized by loss-of-function or reduced expression or activity of a tumor suppressor gene such as the p53 protein and/or the breast cancer 1 (BRCA1) protein. Cancers treating by the disclosed nanoparticle compositions and methods may include, but are not limited to, uterine cancers and breast cancers.

The disclosed nanoparticle compositions may comprise one or more of the following as components: (a) one or more cytotoxic and/or chemotherapeutic drugs; (b) biodegradable and/or biocompatible nanoparticles; optionally (c) a surfactant; and optionally (d) liposomes and/or components for forming liposomes. Suitable cytotoxic and/or chemotherapeutic drugs may include but are not limited to cytoskeletal drugs, anti-angiogenic drugs, inhibitors of poly ADP-ribose polymerases 1 and 2 (PARP inhibitors), inhibitors of the p38 mitogen-activated protein kinase (MAPK) pathway, and/or combinations thereof.

Also disclosed herein are methods for treating cancer in a subject in need thereof. The disclosed methods may include administering to a subject in need thereof a composition comprising nanoparticles, which preferably are biodegradable and/or biocompatible, and a combination of agents that induce synthetic lethality.

In particular, the disclosed methods may be utilized to treat cancers characterized by loss-of-function of the p53 protein and/or loss-of-function of the BRCA1 protein, the method comprising administering to the subject a pharmaceutical composition as disclosed herein. Cancers treated by the disclosed methods may include, but are not limited to, cancers selected from cancer of the following: adrenal gland, bladder, bone, bone marrow, brain, breast, cervix, gall bladder, ganglia, gastrointestinal tract, heart, kidney, liver, lung, muscle, ovary, pancreas, parathyroid, prostate, skin, testis, thymus, and uterus. The methods may be utilized to treat uterine cancers such as endometrial cancers, and in particular, uterine serous carcinoma.

### BRIEF DESCRIPTION OF THE DRAWINGS

FIG. 1(a), FIG. 1(b), and FIG. 1(c): Concomitant treatment of PTXs+BIBFs significantly inhibited cell growth only in EC cells with LOF p53 mutations. FIG. 1(a) Three EC cell lines were treated with PTXs and/or BIBFs for 72 h: Ishikawa cells; 5 nM PTXs and 2.5 μM BIBFs, Hec50co cells; 5 nM PTXs and 2.5 μM BIBFs, and KLE cells; 10 nM PTXs and 2.5 μM BIBFs. All combinatorial treatments were concomitant. FIG. 1(b) Sequential and concomitant treatments were also evaluated using Hec50co cells. PTXs and BIBFs doses were the same as in FIG. 1(a). The first treatment was added for 48 h, washed away, and then the second treatment was added for an additional 72 h. The untreated control group was incubated with fresh media for 5 days. FIG. 1(c) Synergy between PTXs and BIBFs was evaluated in Hec50co cells. Left panel represents dose response curves of PTXs, BIBFs or the combination using varied concentrations of PTXs with either 1 μM BIBFs or 100 nM BIBFs for 72 h. Right panel represents combination index (CI) vs. fraction affected (Fa) curve; CI<1 indicates synergy. Cytotoxicity was determined using the MTS assay. Statistical analysis for panels A and B was performed using one-way ANOVA with Tukey post hoc test. Data are expressed as mean ±SEM (n=3). ***p<0.001, *p<0.05.

FIG. 2(a), FIG. 2(b), FIG. 2(c), FIG. 2(d), FIG. 2(e), and FIG. 2(f): PTXp were successfully prepared and microscopically characterized. FIG. 2(a) Schematic illustrating the nanoprecipitation method used for nanoparticle preparation. FIG. 2(b) Scanning electron micrographs of PTXp [b-1 to b-4] and Blankp [b-5 to b-8] showing spherically shaped nanoparticles with smooth surfaces. Scale bar=500 nm [100 nm in the insert]. FIG. 2(b-1) PTXp (75/T), FIG. 2(b-2) PTXp (75/P), FIG. 2(b-3) PTXp (50/T), FIG. 2(b-4) PTXp (50/P), FIG. 2(b-5) Blankp (75/T), FIG. 2(b-6) Blankp (75/P), FIG. 2(b-7) Blankp (50/T), FIG. 2(b-8) Blankp (50/P). FIG. 2(c) Confocal microscopy images of Hec50co cells incubated with 4 different RHDp for 4 h. Blue: nucleus (DAPI), red: plasma membrane (cell mask deep red), green: RHDp. Scale bar=50 μm. FIG. 2(d) Z-stacked confocal image of Hec50co cells incubated with RHDp (75/T) for 24 h , utilizing same dyes as in (c). Scale bar=25 μm. FIG. 2(e) Transmission electron micrographs of PTXp (75/T) showing spherical nanoparticles. Scale bar=500 nm [100 nm in the insert]. FIG. 2(f) Transmission electron micrographs of Hec50co cells showing the uptake of PTXp (75/T) (black arrows) following 24 h incubation. Scale bar=200 nm.

FIG. 3(a), FIG. 3(b), FIG. 3(c), FIG. 3(d), and FIG. 3(e): PTXp (75/T) exhibited highest cell killing and uptake against Hec50co cells, in addition to slower drug release. FIG. 3(a) Cytotoxicity associated with the use of different PTXp formulations against three EC cell lines after 72 h of incubation. PTX dose: 5 nM in both Ishikawa and Hec50co cells, and 10 nM in KLE cells. Doses were selected based on the sensitivity of each cell line to PTX, in a way that ˜75% cell viability is achieved with PTXs (see FIG. 7). FIG. 3(b) Dose response curve of different PTXp formulations against the three EC cell lines after 72 h of incubation. In both experiments FIG. 3(a) and FIG. 3(b), PTXp (75/T) and PTXp (75/P) were prepared on the first day, stored overnight at 4° C., and then PTXp (50/T) and PTXp (50/P) were prepared on the second day, when all the treatments were initiated. FIG. 3(c) Cytotoxicity associated with the use of different Blankp formulations against three EC cell lines after 72 h of incubation. Doses of the Blankp were equivalent to 5 nM and 100 nM in the PTXp formulation. FIG. 3(d) Flow cytometry analysis for uptake studies of different RHDp formulations against three EC cell lines after 6 h of incubation in serum free media. Upper panels show histograms of different treatments. Lower panels show median fluorescence intensity of these histograms. FIG. 3(e) Release studies of different PTXp formulations in 1% v/v Tween 80 solution in phosphate buffered saline. Cytotoxicity in FIG. 3(a), FIG. 3(b) and FIG. 3(c) was determined using the MTS assay. Statistical analysis was performed using one-way ANOVA with Tukey post hoc test. Data are expressed as mean±SEM (n=3). ***p<0.001, **p<0.01.

FIG. 4(a), FIG. 4(b), FIG. 4(c), FIG. 4(d), and FIG. 4(e): BIBFs induced synthetic lethality to PTXp (75/T) in LOF p53 cells through the abrogation of the G2/M checkpoint. FIG. 4(a) Cell cycle profiles of Hec50co cells treated with either 1 μM BIBFs, 40 nM PTXp (75/T), or the combination of both for 24 h. The percentage of cells in G2/M transition is indicated in red in each plot. FIG. 4(b) Western blot analysis showing the effect of either 1 μM BIBFs, 40 nM PTXp (75/T), or the combination of both on the post translational modification of cell cycle regulators in Hec50co cells following 24 h incubation. * represents a slow migrating band of phosphorylated CDC25C. FIG. 4(c) Cytotoxicity associated with the use of 1 μM BIBFs, 5 nM of PTXp (75/T), or the combination of both against Hec50co cells and GOF Hec50co cells, following 72 h incubation. Cytotoxicity was assessed using MTS assay. FIG. 4(d) and FIG. 4(e) The effect of 1 μM BIBFs on the uptake of different RHDp formulations after 6 h incubation with FIG. 4(d) Hec50co cells, or FIG. 4(e) GOF Hec50co cells, as determined by flow cytometry. Upper panels show histograms of different treatments, while lower panel shows median fluorescence intensity data of these histograms. Statistical analysis was performed using one-way ANOVA with Tukey post hoc test. Data are expressed as mean±SEM (n=3) in FIG. 4(c), FIG. 4(d) and FIG. 4(e). ***p<0.001, **p<0.01.

FIG. 5(a), FIG. 5(b), FIG. 5(c), FIG. 5(d), FIG. 5(e), FIG. 5(f), and FIG. 5(g): The combination of PTXp (75/T) +BIBFp (75/T) demonstrated highest reduction in tumor progression, extended median survival and favorable safety in vivo. FIG. 5(a) Cytotoxicity associated with the use of 100 nM BIBFs or 100 nM BIBFp (75/T) in combination with different PTX concentrations against Hec50co cells, as measured by MTS assays. Indicated treatments involved incubation with cells for 72 h. Statistical analysis was performed using one-way ANOVA with Tukey post hoc test. Data are expressed as mean±SEM (n=3). ***p<0.001, **p<0.01, *p<0.05. FIG. 5(b) Tumor progression curves in athymic NCI-nu/nu mice challenged subcutaneously with 2×10⁶ Hec50co cells in the right flank. Mice were treated with either saline (naive), 5 mg/kg PTXs, 5 mg/kg PTXp (75/T), or the combination therapy of 5 mg/kg PTXp (75/T) and 5 mg/kg BIBFp (75/T). Treatments were administered IV through retro-orbital injections in the venous sinus on days 18, 25, and 32. Statistical analysis was performed using a non-parametric Kruskal-Wallis test. Data are presented as mean±SEM (n=7 for combination group, otherwise, n=5). *p<0.05, ***p<0.001. FIG. 5(c) Representative photographs of tumors (black dotted circles) on day 32 post tumor challenge. FIG. 5(d) Kaplan-Meier survival curves comparing variously treated mice with the naive group. Values of median survival is shown in brackets. Statistical analysis was performed using the Log-rank test with Bonferroni post hoc test. *p<0.05 compared to the naive group. FIG. 5(e) Mice weight change over time during treatments. Mice were weighted on days 18, 25 and 32. Data are presented as mean±SEM. FIG. 5(f) H & E staining of mice organs collected after euthanizing the treated mice (mice were treated as described in FIG. 5(b)). Mice were euthanized when their tumor dimensions reached 2 cm in length or width, or 1 cm in height. Images were captured using 100× lens. Scale bar=40 μm. FIG. 5(g) Intra-tumoral PTX concentration over a 12 h period following single IV (retro-orbital) injection of either 5 mg/kg PTXs or 5 mg/kg PTXp (75/T) quantified using a validated LC-MS/MS method (see Supplementary Information). Statistical analysis was performed using unpaired two-tailed t-test. Data are expressed as mean±SEM (n=3). **p<0.01.

FIG. 6: BIBF target FGFR2 is expressed in three endometrial cancer cell lines: Hec50co, Ishikawa and KLE. Representative western blot depicting FGFR2 expression. β-actin, loading control. Cells were also screened for the presence of FGFR2 activating mutations, which occur in ˜10-16% of endometrial cancers. Previous reports have established that KLE and Ishikawa cells contain WT FGFR2. To confirm the published data and to determine if Hec50co cells contain WT or activated FGFR2, mutational hotspot regions in the third immunoglobulin domain (IIIC) and the transmembrane domain of FGFR2 were sequenced in the three cell lines. No mutations in FGFR2 were detected, indicating that all three cell lines contain WT FGFR2.

FIG. 7: Dose response curves of three EC cell lines. Indicated cells were incubated with soluble forms of either drug alone for 72 h, and cytotoxicity was evaluated using the MTS cell proliferation assay. Data are expressed as mean±SEM (n=3).

FIG. 8: Significantly increased RHD uptake was observed when blood-brain barrier (hCMEC/D3) cells were treated with RHDp (75/T) versus RHDs. Cells were incubated with either 0.01 μg of RHDs or RHDp (75/T) for 6 h in serum free media, and then uptake was evaluated using flow cytometry. Left, representative histograms of different treatments. Right, bar chart summarizing the median fluorescence intensity of each treatment. Statistical analysis was performed using one-way ANOVA with Tukey post hoc test. Data are expressed as mean±SEM (n=3). ***p<0.001.

FIG. 9: PTXp (75/T) was significantly more cytotoxic than PTXs against the PTX-resistant cell line, LLC-PK1-MDR1. Left, LLC-PK1-WT cells. Right, LLC-PK1-MDR1 cells. Cells were incubated with different concentrations of PTXs, PTXp (75/T), Blankp (75/T) for 72 h, and cytotoxicity was evaluated using the MTS cell proliferation assay. Statistical analysis was performed using one-way ANOVA with Tukey post hoc test. Data are expressed as mean±SEM (n=3). ***p<0.001.

FIG. 10(a), FIG. 10(b), FIG. 10(c), FIG. 10(d), FIG. 10(e), and FIG. 10(f): PTXp (75/T)-induced cytotoxicity against Hec50co cells is demonstrated by inhibition of cell proliferation, decreased DNA content, decreased number of viable cells, increased cellular ATP content, increased apoptosis, and increased cells undergoing mitosis. In these set of experiments, cells were incubated with either 5 nM PTXs, 5 nM PTXp (75/T), or Blankp (75/T)=5 nM for 24 h only, in order to maintain sufficient live cells to effectively perform each assay. FIG. 10(a) Cell viability was assessed using the MTS cell proliferation assay. FIG. 10(b) DNA content was estimated using the CyQUANT® direct cell proliferation assay. FIG. 10(c) Viable cell count was evaluated using trypan blue staining. FIG. 10(d) ATP content was estimated using the ATP assay kit. FIG. 10(e) Apoptosis (%) was evaluated using flow cytometry after staining the cells with Annexin V/PI (left panel), and the total percentage of cells in early and late apoptosis was calculated by summing the (%) of cells in both Q1 and Q2 (right panel). FIG. 10(f) Cells undergoing mitosis (rounded cells) were imaged using bright field microscopy utilizing 10× lens. Scale bar=500 μm. Statistical analysis was performed using one-way ANOVA with Tukey's post hoc test. Data are expressed as mean±SEM (n=3). *p<0.05.

FIG. 11: Scanning electron micrograph of BIBFp (75/T) showing spherical nanoparticles with smooth surfaces. Scale bar=1 μm.

FIG. 12(a), FIG. 12(b), FIG. 12(c), FIG. 12(d), FIG. 12(e), and FIG. 12(f): LC-MS/MS method validation for intra-tumoral PTX quantification FIG. 12(a) MS/MS spectra of PTX and fragmentation pattern of PTX with product ions m/z 696.30,569.20, 509.20,387.20 and 286.15, FIG. 12(b) MS/MS spectra of PTX-d5 (IS) with product ions m/z 569.20, 509.20,387.20 and 291.15. FIG. 12(c) & FIG. 12(d) Representative MRM ion- overlay chromatograms of FIG. 12(c) blank tumor homogenate and standard spiked PTX at 1.0 ng/mL, and FIG. 12(d) blank tumor homogenate and IS spiked PTX-d5 at 100 ng/mL. FIG. 12(e) & FIG. 12(f) Calibration curves in FIG. 12(e) neat solution and FIG. 12(f) tumor homogenate.

FIG. 13(a) and FIG. 13(b): Between 10-15% of the total DIRp (75/T) dose accumulated in the tumors of mice 48 h post IV injection. The biodistribution of DIRp (75/T) was assessed in three different murine tumor models. FIG. 13(a) This panel shows the IVIS fluorescence images of DIRp (75/T) in the organs of mice 48 h post injection. In each tumor model, an untreated mouse served as the control. FIG. 13(b) This panel shows a summary of fluorescence intensities of each organ normalized to the total fluorescence intensity of all organs (see methods and materials for details) in the various tumor models.

### DETAILED DESCRIPTION

Disclosed are compositions, kits, and methods for treating cancer in a subject in need thereof, in particular in a subject having a cancer characterized by solid tumors. The compositions, kits, and methods may be further described as follows.

Unless otherwise specified or indicated by context, the terms “a”, “an”, and “the” mean “one or more.” In addition, singular nouns such as “cytotoxic drug,” should be interpreted to mean “one or more cytotoxic drugs,” unless otherwise specified or indicated by context.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The terms “subject,” “patient,” or “host” may be used interchangeably herein and may refer to human or non-human animals. Non-human animals may include, but are not limited to non-human primates, dogs, cats, horses, or other non-human animals.

The terms “subject,” “patient,” or “individual” may be used to refer to a human or non-human animal having or at risk for acquiring a cell proliferative disease or disorder. Subjects who are treated with the compositions disclosed herein may be at risk for cancer or may have already acquired cancer including cancers characterized by solid tumors. Cancers characterized by solid tumors may include, but are not limited to adenocarcinoma, lymphoma, melanoma, myeloma, sarcoma, and teratocarcinoma and particularly cancers of the adrenal gland, bladder, bone, bone marrow, brain, breast, cervix, gall bladder, ganglia, gastrointestinal tract, heart, kidney, liver, lung, muscle, ovary, pancreas, parathyroid, prostate, skin, testis, thymus, and uterus.

Cell proliferative diseases or disorders may include cancers characterized by loss-of-function (LOF) of the p53 protein. In particular, cancers contemplated herein may include uterine cancers that are characterized by LOF of the p53 protein, such as uterine serous carcinoma.

Cell proliferative diseases or disorders may include cancers characterized by loss-of-function (LOF) of the breast cancer 1 (BRCA1) protein. In particular, cancers contemplated herein may include breast cancers that are characterized by LOF of the BRCA1 protein.

The disclosed nanoparticle compositions and methods may comprise and/or utilize one or more of the following as components: (a) one or more cytotoxic and/or chemotherapeutic drugs; (b) biodegradable and/or biocompatible nanoparticles; optionally (c) a surfactant; and optionally (d) liposomes and/or components of liposomes. Suitable cytotoxic and/or chemotherapeutic drugs may include but are not limited to cytoskeletal drugs, anti-angiogenic drugs, inhibitors of poly ADP-ribose polymerases 1 and 2 (PARP inhibitors), inhibitors of the p38 mitogen-activated protein kinase (MAPK) pathway, and/or combinations thereof.

The disclosed compositions and methods include or utilize a cytoskeletal drug. Cytoskeletal drugs are known in the art and may include small molecules that interact with actin or tubulin and may prevent mitosis, for example by stabilizing microtubules comprising tubulin. Cytoskeletal drugs may include, but are not limited to, paclitaxel (PTX)(i.e., brand name Taxol®) or derivatives of PTX such as docetaxel (see also “The Chemistry and Pharmacology of Taxol® and its Derivatives,” Volume 22, 1^{st} Edition, Editors: V. Farina; Authors: H. Timmerman, 1995). Other cytoskeletal drugs may include, but are not limited to demecolcine, vinblastine, colchicine, cytochalasin, latrunculin, jasplakinolid, nocodazole, phalloidin, swinholide, and rotenone.

The disclosed compositions and methods include or utilize an anti-angiogenic drug. Anti-angiogenic drugs are known in the art and may include tyrosine kinase inhibitors that inhibit the activity of one or more receptors selected from the group consisting of vascular endothelial growth factor receptor (VEGFR), fibroblast growth factor receptor (FGFR), platelet-derived growth factor receptor (PDGFR), or any combination thereof. Anti-angiogenic drugs may include, but are not limited to, BIBF-1120 (i.e., nintedanib), sorafenib (e.g., brand name Nexavar®), sunitinib (e.g., brand name Sutent®), and pazopanib (e.g., brand name Votrient®). Preferably, the anti-angiogenic drug of the disclosed compositions and methods inhibits the P-glycoprotein efflux transporter (P-gp).

The disclosed compositions and methods include or utilize inhibitors of poly ADP-ribose polymerases 1 and 2 (PARP inhibitors). PARP inhibitors may include, but are not limited to, BT-888 (Veliparib, XAV-939, A4164 AZD2461, A4159 PJ34 hydrochloride, A4158 AG-14361, A4157 Iniparib (BSI-201), A4156 Rucaparib (AG-014699,PF-01367338), A4154 Olaparib (AZD2281, Ku-0059436), A4153 BMN 673, A8893 Rucaparib (free base), A8808 ME0328, A8601 Tankyrase Inhibitors (TNKS) 49, A8600 Tankyrase Inhibitors (TNKS) 22, A4529 JW 55, A3729 PJ34, A4161 INO-1001, A4531 WIKI4, A4530 NU 1025, A4527 DR 2313, A4526 BYK 49187, A4525 BYK 204165, A3617 MK-4827, A3246 BMN-673 8R,9S, A4163 UPF 1069, A4160 A-966492, A4524 4-HQN, A4528 EB 47, B1163 MK-4827 hydrochloride, B1164 MK-4827 tosylate, B3393 MK-4827 Racemate, A3958 Veliparib dihydrochloride, and combinations thereof.

The disclosed compositions and methods include or utilize inhibitors of the p38 mitogen-activated protein kinase (MAPK) pathway. Inhibitors of the p38 mitogen-activated protein kinase (MAPK) pathway may include, but are not limited to, SB203580, Doramapimod (BIRB 796), SB202190 (FHPI, LY2228820 VX-702, Pamapimod (R-1503, Ro4402257, PH-797804, VX-745, TAK-715, SB239063, Skepinone-L, Losmapimod (GW856553X, Asiatic Acid, BMS-582949, Pexmetinib (ARRY-614), and combinations thereof. In some embodiments of the disclosed methods, a subject in need thereof is administered a dose of an inhibitor of the p38 MAPK pathway that is relatively lower than a dose administered to a subject in conventional treatment methods. For example, in the disclosed methods, as subject may be administered a dose of an inhibitor of the p38 MAPK pathway that is less than about 200, 100, 90, 80, 70, 60, 50, 40, 30, 20, or 10 mg, or a dose within a range bounded by any of these values (e.g., 50-100 mg).

The disclosed compositions and methods include or utilize biodegradable and/or biocompatible nanoparticles. The disclosed nanoparticles typically have an effective diameter of less than 500 μm, and preferably have an effective diameter of less than 400, 300, 200, 150, 100, or 50 μm, or have an effective diameter within a range bounded by any of these values (e.g., an effective diameter within a range of 50-200 μm).

The nanoparticles disclosed herein may comprise a biodegradable polymer as would be understood in the art. The term “biodegradable” describes a material that is capable of being degraded in a physiological environment into smaller basic components such as organic polymers. Preferably, the smaller basic components are innocuous. For example, a biodegradable polymer may be degraded into basic components that include, but are not limited to, water, carbon dioxide, sugars, organic acids (e.g., tricarboxylic or amino acids), and alcohols (e.g., glycerol or polyethylene glycol). Biodegradable polymers that may be utilized to prepare the particles contemplated herein may include materials disclosed in U.S. Pat. Nos. 7,470,283; 7,390,333; 7,128,755; 7,094,260; 6,830,747; 6,709,452; 6,699,272; 6,527,801; 5,980,551; 5,788,979; 5,766,710; 5,670,161; and 5,443,458; and U.S. Published Application Nos. 20090319041; 20090299465; 20090232863; 20090192588; 20090182415; 20090182404; 20090171455; 20090149568; 20090117039; 20090110713; 20090105352; 20090082853; 20090081270; 20090004243; 20080249633; 20080243240; 20080233169; 20080233168; 20080220048; 20080154351; 20080152690; 20080119927; 20080103583; 20080091262; 20080071357; 20080069858; 20080051880; 20080008735; 20070298066; 20070288088; 20070287987; 20070281117; 20070275033; 20070264307; 20070237803; 20070224247; 20070224244; 20070224234; 20070219626; 20070203564; 20070196423; 20070141100; 20070129793; 20070129790; 20070123973; 20070106371; 20070050018; 20070043434; 20070043433; 20070014831; 20070005130; 20060287710; 20060286138; 20060264531; 20060198868; 20060193892; 20060147491; 20060051394; 20060018948; 20060009839; 20060002979; 20050283224; 20050278015; 20050267565; 20050232971; 20050177246; 20050169968; 20050019404; 20050010280; 20040260386; 20040230316; 20030153972; 20030153971; 20030144730; 20030118692; 20030109647; 20030105518; 20030105245; 20030097173; 20030045924; 20030027940; 20020183830; 20020143388; 20020082610; and 0020019661; the contents of which are incorporated herein by reference in their entireties. Typically, the biodegradable nanoparticles disclosed herein are degraded in vivo at a degradation rate such that the nanoparticles lose greater than about 50%, 60%, 70%, 80%, 90%, 95%, or 99% of their initial mass after about 1, 2, 3, 4, 5, 6, 7, or 8 weeks post-administration to a subject in need thereof via one or more of: degradation of the biodegradable polymers of the nanoparticles to monomers: degradation of the biodegradable polymers of the nanoparticles to water, carbon dioxide, sugars, organic acids (e.g., tricarboxylic or amino acids), and alcohols (e.g., glycerol or polyethylene glycol); and degradation of the nanoparticles to release a drug contained in the nanoparticles or any other active agent of the nanoparticles.

Suitable polymers for preparing the nanoparticles may include, but are not limited to, polymers such as polylactides (PLA), including polylactic acid, polyglycolides (PGA), including polyglycolic acid, and co-polymers of PLA and PGA, for example, poly(lactic-co-glycolic acid (PLGA). The concentration of PLA and PGA may be varied, for example, PLGA 75:25 having 75% PLA and 25% PGA, or PLGA 50:50 having 50% PLA and 25% PGA. Other suitable polymers may include, but are not limited to, polycaprolactone (PCL), poly(dioxanone) (PDO), collagen, renatured collagen, gelatin, renatured gelatin, crosslinked gelatin, and their co-polymers. The selected polymer(s) may be of any suitable molecular weight. The polymer of the nanoparticles may be designed to degrade as a result of hydrolysis of polymer chains into biologically acceptable and progressively smaller components (e.g., such as polylactides, polyglycolides, and their copolymers, which may break down eventually into lactic and glycolic acid, enter the Kreb's cycle, be broken down into carbon dioxide and water, and excreted).

The disclosed nanoparticles may comprise a biocompatible polymer as known in the art. Suitable biocompatible polymers may include, but are not limited to silk, elastin, chitin, chitosan, poly(d-hydroxy acid), poly(anhydrides), and poly(orthoesters). More particularly, the biocompatible polymer may comprises polyethylene glycol, poly(lactic acid), poly(glycolic acid), copolymers of lactic and glycolic acid, copolymers of lactic and glycolic acid with polyethylene glycol, poly(E-caprolactone), poly(3-hydroxybutyrate), poly(p-dioxanone), polypropylene fumarate, poly(orthoesters), polyol/diketene acetals addition polymers, poly(sebacic anhydride) (PSA), poly(carboxybiscarboxyphenoxyphenoxy hexone (PCPP) poly[bis (p-carboxypheonoxy) methane] (PCPM), copolymers of SA, CPP and CPM, poly(amino acids), poly(pseudo amino acids), polyphosphazenes, derivatives of poly[(dichloro)phosphazenes] and poly[(organo) phosphazenes], polysulfenamides, poly-hydroxybutyric acid, or S-caproic acid, polylactide-co-glycolide, polylactic acid, polyethylene glycol, and/or combinations thereof.

The disclosed nanoparticles may be prepared by methods known in the art. In some embodiments, the nanoparticles may be formed from a solution or suspension of a polymer in the presence of one or more drugs or cytotoxic and/or chemotherapeutic drugs (e.g., a cytoskeletal drug and/or an anti-angiogenic drug). As such, the nanoparticles may comprise a polymer and one or more drugs as contemplated herein.

The nanoparticles may comprise a suitable concentration of the drug for treating cancer in a subject in need thereof. In some embodiments, the nanoparticles may comprise the drug at concentration value of at least about 0.01, 0.02, 0.05, 0.1, 0.2, 0.5, 1, 2, 5, 10, 20, 30, 40, 50, 100, or 200 μg/mg; or the nanoparticles may comprise the drug at a concentration value of no more than about 200, 100, 50, 20, 10, 5, 2, 1, 0.5, 0.2, 0.1, 0.05, 0.02 μg/mg; or the nanoparticles may comprise the drug within a concentration range bounded by any of the preceding concentration values (e.g. within a concentration range of 30-50 μg/mg).

In particular, the nanoparticles comprise a cytoskeletal drug (e.g., PTX) at a concentration of at least about 5, 10, 20, 30, 40, 50, 100, or 200 μg/mg nanoparticle or within a concentration range bounded by any of these values (e.g., 30-50 μg/mg nanoparticle).

In particular, the nanoparticles comprise an anti-angiogenic drug (e.g., BIBF-1120) at a concentration of at least about 5, 10, 20, 30, 40, 50, 100, or 200 μg/mg nanoparticle or within a concentration range bounded by any of these values (e.g., 30-50 μg/mg nanoparticle).

In some embodiments, the nanoparticles comprise a cytoskeletal drug (e.g., PTX) and an anti-angiogenic drug (e.g., BIBF-1120). The nanoparticles may comprise the cytoskeletal drug (e.g., PTX) and the anti-angiogenic drug (e.g. BIBF-1120) at a suitable molar concentration ratio (e.g., PTX:BIBF-1120). Suitable molar ratios of the cytoskeletal drug (e.g., PTX) and the anti-angiogenic drug (e.g. BIBF-1120) in the nanoparticles may include molar ratios (e.g., PTX:BIBF-1120) selected from the group consisting of 0.05:1, 0.1:1, 0.2:1, 0.3:1, 0.4:1, 0.5:1 0.6:1, 0.7:1, 0.8:1, 0.9:1, 1:1, 1:0.9, 1:0.8, 1:0.7, 1:0.6, 1:0.5, 1:0.4, 1:0.3, 1:0.2, 1:0.1, 1:0.05 or within a molar concentration ratio range bounded by any of these molar concentration ratios (e.g., a molar concentration ratio range of 1:(0.2-0.5)).

The disclosed pharmaceutical compositions may include a surfactant. In some embodiments, the pharmaceutical compositions include a surfactant and are formulated as a suspension of the nanoparticles and/or an emulsion comprising the nanoparticles and any other components of the pharmaceutical compositions as contemplated herein. Surfactants for formulating pharmaceutical suspensions and/or emulsions are known in the art. In some embodiments, the surfactant comprises a water soluble polymer (e.g., polyethylene glycol or polyvinyl alcohol) optionally coupled to a hydrophobic molecule (e.g., a methylated phenyl compound such as a tocopherol, and in particular vitamin E or a derivative thereof). In particular, a suitable surfactant may include a polyethylene glycol coupled to a tocopherol, such as D-α-tocopherol glycol 1000 succinate (i.e., TPGS).

In some embodiments, the surfactant of the disclosed compositions and methods inhibits the P-glycoprotein efflux transporter (P-gp). (See, e.g., Hoosain et al., “Bypassing P-Glycoprotein Drug Efflux Mechanisms: Possible Appilations in Pharacoresistant Schizophrenia Therapy, Biomed Res Int. 2015; 2015: 484963, Published on-line 2015 Sep. 27; the the content of which is incorporated herein by reference in its entirety). As discussed in Hoosain et al., surfactants (and solvents) act by interacting with the polar heads of the lipid bilayers of cells and have the potential to insert themselves between the nonpolar tails of the lipid bilayers, causing increased fluidization of the lipid membrane and P-gp inhibition. Nonionic surfactants such as Tween and Span possess P-gp transporter inhibitory potential and also hydrophobic and thus rendered less toxic. (See, e.g.,, Bansal et al., “Novel formulation approaches for optimising delivery of anticancer drugs based on P-glycoprotein modulation,” Drug Discovery Today. 2009;14(21-22):1067-1074; the content of which is incorporated herein by reference in its entirety). Research has shown that the efficiency of surfactants as P-gp inhibitors is based on their respective chemical structures. Surfactants such as Solutol HS15, Tween 80, and Cremaphore EL, which contain polyethylene glycol on the hydrophilic portions of their structures, display the ability to increase intracellular concentrations of epirubicin in human colorectal carcinoma cells, thereby confirming that these surfactants act as P-gp modulators. (See, e.g., Nieto Montesinos et al., “Delivery of P-glycoprotein substrates using chemosensitizers and nanotechnology for selective and efficient therapeutic outcomes,” Journal of Controlled Release. 2012;161(1):50-61; the content of which is incorporated herein by reference in its entirety). In addition, Tween 80, Cremophor EL, and vitamin E TPGS have been shown to inhibit P-gp. (See, e.g., Rege et al., “Effects of nonionic surfactants on membrane transporters in Caco-2 cell monolayers,” European Journal of Pharmaceutical Sciences. 2002;16(4-5):237-24; the content of which is incorporated herein by reference in its entirety). Tween 80 and Cremophor EL were observed to increase the apical to basolateral permeability of Rhodamine 123, which is a P-gp substrate, within a concentration range of 0-1 mM, whereas vitamin E TPGS inhibited the apical to basolateral permeability of Rhodamine 123 at a concentration of 0.025 mM. (See id.). Additional suitable surfactants for the the disclosed compositions and methods which may act as inhibitors of P-pg may include, but are not limited to polymers that include D-mannose monomers such as xanthan gum, gellan gum, alginates, and/or combinations thereof. (See, e.g., Hunter et al. “Mechanisms of action of nonionic block copolymer adjuvants,” AIDS Research and Human Retroviruses. 1994;10(2):95-98; the content of which is incorporated herein by reference in its entirety).

In some embodiments, the surfactant of the disclosed compositions and methods may include thiol groups that interact with cysteine residues in the P-gp transmembrane channel forming disulfide bondins and blocking efflux through the P-gp transmembrane channel. Additional suitable surfactants for the disclosed compositions and methods may include, but are not limited to, thiomers. (See, e.g., Batrakova, et al, “Pluronic P85 enhances the delivery of digoxin to the brain: in vitro and in vivo studies,” The Journal of Pharmacology and Experimental Therapeutics. 2001;296(2):551-557; the content of which is incorporated herein by reference in its entirety).

In some embodiments, the surfactant of the disclosed compositions and methods changes the microenvironment of cell membranes (e.g., Caco-2 cell membranes) leading to modification in membrane fluidity. Additional suitable surfactants for the disclosed compositions and methods may include, but are not limited to, polyethylene glycol 300, polyethylene glycol 400, polyethylene glycol-poly(ethylene imine), which optionally are functionalized. (See, e.g., Werle M., “Natural and synthetic polymers as inhibitors of drug efflux pumps,” Pharmaceutical Research. 2008;25(3):500-511; the content of which is incorporated herein by reference in its entirety).

In some embodiments, the surfactant of the disclosed compositions and methods results in ATPase inhibition and/or ATPase reduction, as well as membrane fluidization. Additional suitable surfactants for the disclosed compositions and methods may include, but are not limited to, pluronic surfactants such as pluoronic P85. (See, e.g., Hugger Eet al., “Effects of poly(ethylene glycol) on efflux transporter activity in Caco-2 cell monolayers,” Journal of Pharmaceutical Sciences. 2002;91(9):1980-1990; and Johnson et al., “An in vitro examination of the impact of polyethylene glycol 400, pluronic p85, and vitamin E d-a-tocopheryl polyethylene glycol 1000 succinate on p-glycoprotein efflux and enterocyte-based metabolism in excised rat intestine,” The AAPS Journal. 2002;4(4):193-205; the contents of which are incoporated herein by reference in their entireties).

The disclosed pharmaceutical compositions may include liposomes and/or components of liposomes. The use of liposomes in drug delivery systems is known in the art. (See, e.g.,, Alavi et al., “Application of Various Types of Liposomes in Drug Delivery Systems,” Adv. Pharm. Bull. 2017 Apr;7(1):3-9, the content of which is incorporated herein by reference in its entirety).

The disclosed pharmaceutical compositions may include additional components. In some embodiments, the disclosed pharmaceutical compositions further comprise a T-cell stimulatory agent, optionally wherein the nanoparticles of the pharmaceutical composition comprise the T-cell stimulatory agent, and optionally wherein the T-cell stimulatory agent is a TLR agonist which is selected from the group consisting of unmethylated CpG dinucleotide (CpG-ODN), polyribosinic:polyribocytidic acid (Poly I:C), polyadenosine-polyruridylilc acid (poly AU), polyinosinic-polycytidylic acid stabilized with poly-L-lysine and carboxymethylcellulose (Poly-ICLC), bacterial lipopolysaccharides (e.g., monophosphoryl lipid A (MPL)), MUC1 mucin (e.g., Sialyl-Tn (STn)), and imidazoquinolines (e.g., imiquimod and resiquimod), or optionally wherein the T-cell stimulatory agent targets a TNFR costimulatory molecule and is selected from a group consisting of an anti OX40 agonist antibody, an anti CD40 agonist antibody, an anti CD137 agonist antibody.

In some embodiments, the disclosed pharmaceutical compositions further comprise an immune checkpoint inhibitor, optionally wherein the nanoparticles of the pharmaceutical composition comprise the immune checkpoint inhibitor, and optionally wherein the immune checkpoint inhibitor is selected from the group consisting of an anti CTLA-4 antibody (e.g., Ipilimumab or Tremelimumab), an anti PD-1 antibody (MDX-1106, BMS-936558, MK3475, CT-011, AMP-224), an anti PD-L1 antibody (e.g., MDX-1105), an anti IDO-1 antibody, and anti IDO-2 antibody, an anti KIR antibody, an anti CD70 antibody, an anti LAG-3 antibody (e.g., IMP321), an anti B7-H3 antibody (e.g., MGA271), and anti B7-H4 antibody, an anti TIM3 antibody, and combinations thereof.

A specific pharmaceutical composition contemplated herein may comprise the following components: (a) PTX; (b) BIBF-1120; (b) nanoparticles; and (d) TPGS. In this specific pharmaceutical composition, the nanoparticles may comprise PTX, BIBF-1120, or both of PTX and BIBF-1120, at suitable concentrations as disclosed herein and/or at suitable molar ratios as contemplated herein.

Also contemplated herein are methods for treating a subject having cancer. Suitable cancers treated by the disclosed methods may include, but are not limited to, cancers characterized by loss-of-function of the p53 protein and/or loss-of-function of the breast cancer 1 (BRCA1) protein. The methods may include administereing to the subject any pharmaceutical compositions as contemplated herein. Suitable cancers treated by the disclosed methods may include, but are not limited to cancers selected from the group consisting of cancers of the adrenal gland, bladder, bone, bone marrow, brain, breast, cervix, gall bladder, ganglia, gastrointestinal tract, heart, kidney, liver, lung, muscle, ovary, pancreas, parathyroid, prostate, skin, testis, thymus, and uterus. In particular, the disclosed methods may be utilized to treat a cancer of the uterus (e.g., endometrial cancer) such as uterine serous carcinoma (USC).

In the disclosed methods for treating a subject having cancer, in some embodiments the methods may include steps of (a) administering to the subject a cytoskeletal drug (e.g., PTX) that blocks progression of the cancer cells through mitosis; and (b) administering to the subject an anti-angiogenic drug (e.g., BIBF-1120). In the disclosed methods, the cytoskeletal drug (e.g., PTX) may be administered substantially concurrently with the (e.g., BIBF-1120). The term “substantially concurrently” should be defined to mean that the cytoskeletal drug (e.g., PTX) and the anti-angiogenic drug (e.g., BIBF-1120) are administered to the subject within no more than 1 hour of each, and preferably within no more than 30, 20, 10, 5, 4, 3, 2, or 1 minutes of each other, or preferably where the cytoskeletal drug (e.g., PTX) and the anti-angiogenic drug (e.g., BIBF-1120) are present in a single pharmaceutical composition that is administered to the subject.

In the disclosed methods for treating a subject having cancer, the subject may be administered an effective dose of a cytoskeletal drug (e.g., PTX). For example, the cytoskeletal drug (e.g., PTX) may be formulated as nanoparticles comprising the cytoskeletal drug, which are administered to deliver at least about 10, 20, 50, 100, 150, 200, 250 mg of the cytoskeletal drug or higher. In another example, the cytoskeletal drug (e.g., PTX) may be formulated as nanoparticles comprising the cytoskeletal drug, which are administered to deliver no more than about 250, 200, 100, 50, 20, 05 10 mg of the cytoskeletal drug or less. In another example, the cytoskeletal drug (e.g. PTX) may be formulated as nanoparticles comprising the cytoskeletal drug, which are administered to deliver a dose of the cytoskeletal drug within a dose range bounded by any of 10, 20, 50, 100, 150, 200, 250 mg (e.g., a dose range of 50-100 mg).

In the disclosed methods for treating a subject having cancer, the subject may be administered an effective dose of an anti-angiogenic drug (e.g., BIBF-1120). For example, the anti-angiogenic drug (e.g., BIBF-1120) may be formulated as nanoparticles comprising the anti-angiogenic drug, which are administered to deliver at least about 10, 20, 50, 100, 150, 200, 250 mg of the anti-angiogenic drug or higher. In another example, the anti-angiogenic drug (e.g., BIBF-1120) may be formulated as nanoparticles comprising the anti-angiogenic drug, which are administered to deliver no more than about 250, 200, 100, 50, 20, 05 10 mg of the anti-angiogenic drug or less. In another example, the anti-angiogenic drug (e.g., BIBF-1120) may be formulated as nanoparticles comprising the angiogenic drug, which are administered to deliver a dose of the anti-angiogenic drug within a dose range bounded by any of 10, 20, 50, 100, 150, 200, 250 mg (e.g., a dose range of 50-100 mg). In the disclosed methods, where a composition is administered to a subject that comprises an anti-angiogenic drug (e.g., BIBF-1120) and a surfactant where the surfactant inhibits the activity of the P-gp efflux transporter, the dose of the anti-angiogenic drug (e.g., BIBF-1120) may be reduced relative to compositions that do not comprise the surfactant that inhibits the activity of the P-gp efflux transporter.

The methods disclosed herein include methods for treating a subject having a cancer susceptible to synthetic lethality, the methods comprising administering to the subject a composition comprising nanoparticles and one or more cytotoxic and/or chemotherapeutic drugs that induce synthetic lethality. The cancer susceptible to synthetic lethality may be characterized by loss-of-function of a tumor suppressor (e.g., the tumor suppressor is p53 or breast cancer protein 1 (BRCA1)). Cancers treated in the methods may include, but are not limited to, breast cancers, uterine cancers, ovarian cancers, and lung cancers (e.g., non-small cell lung cancers). The cytotoxic and/or chemotherapeutic drugs that are administered in the methods may include, but are not limited to an inhibitor of the poly ADP-ribose polymerase (PARP) 1 or 2 and/or an inhibitor of the p38 mitogen-activated protein kinase (MAPK) pathway.

In particular, the methods disclosed herein may include methods for treating a subject having a cancer characterized by p53 deficiency or downregulation, the methods comprising administering to the subject a pharmaceutical composition comprising nanoparticles, a cytoskeletal drug that block progression of cancers cells through mitosis, and an inhibitor of the p38 MAPK pathway, wherein a dose of the inhibitor of the p38 MAPK pathway of less than about 200, 150, 100, 90, 80, 70, 60, 50, 40, 30, 20, or 10 mg is administered to the subject, or a dose within a range bounded by any of these values (e.g., a dose of 50-100 mg). In the methods, the cancer may be characterized by a loss-of-function mutation in p53 and/or a mutation in p53 that reduces the biological activity of p53.

The compositions disclosed herein may be formulated as pharmaceutical composition for administration to a subject in need thereof. Such compositions can be formulated and/or administered in dosages and by techniques well known to those skilled in the medical arts taking into consideration such factors as the age, sex, weight, and condition of the particular patient, and the route of administration.

The compositions may include pharmaceutical solutions comprising carriers, diluents, excipients, and surfactants as known in the art. Further, the compositions may include preservatives. The compositions also may include buffering agents.

The pharmaceutical compositions may be administered therapeutically. In therapeutic applications, the pharmaceutical compositions are administered to a patient in an amount sufficient to elicit a therapeutic effect (e.g., an immune response to a tumor, which eradicates or at least partially arrests or slows growth of the tumor (i.e., a “therapeutically effective dose”)).

The compositions disclosed herein may be delivered via a variety of routes. Typical delivery routes include parenteral administration (e.g., intratumoral, intravenous, intraperitoneal or otherwise). Formulations of the pharmaceutical compositions may include liquids (e.g., solutions and emulsions). The compositions disclosed herein may be co-administered or sequentially administered with other immunological, antigenic or vaccine or therapeutic compositions, including an adjuvant, or a chemical or biological agent given in combination with an antigen to enhance immunogenicity of the antigen. Additional therapeutic agents may include, but are not limited to, cytokines such as interferons (e.g., IFN-γ) and interleukins (e.g., IL-2).

### ILLUSTRATIVE EMBODIMENTS

The following embodiments are illustrative and should not be interpreted to limit the scope of the claimed subject matter.

Embodiment 1. A pharmaceutical composition comprising as components: (a) a cytoskeletal drug that blocks progression of cells through mitosis; (b) an anti-angiogenic drug; (c) nanoparticles, wherein the nanoparticles comprise the cytoskeletal drug, the anti-angiogenic drug, or both of the cytoskeletal drug and the anti-angiogenic drug; (d) optionally a surfactant; and (e) optionally liposomes and/or components of liposomes.

Embodiment 2. The composition of embodiment 1, wherein the cytoskeletal drug is paclitaxel (PTX) or a derivative thereof.

Embodiment 3. The composition of any of the foregoing embodiments, wherein the anti-angiogenic drug is a tyrosine kinase inhibitor that inhibits a receptor selected from the group consisting of vascular endothelial growth factor receptor (VEGFR), fibroblast growth factor receptor (FGFR), platelet-derived growth factor receptor (PDGFR), or any combination thereof.

Embodiment 4. The composition of any of the foregoing embodiments, wherein the anti-angiogenic drug is BIBF-1120.

Embodiment 5. The composition of any of the foregoing embodiments, wherein the nanoparticles comprise the cytoskeletal drug at a concentration of at least about 5, 10, 20, 30, 40, 50, 100, or 200 μg/mg nanoparticle or within a concentration range bounded by any of these values.

Embodiment 6. The composition of any of the foregoing embodiments, wherein the nanoparticles comprise the anti-angiogenic drug at a concentration of at least about 5, 10, 20, 30, 40, 50, 100, or 200 μg/mg nanoparticle or within a concentration range bounded by any of these values.

Embodiment 7. The composition of any of the foregoing embodiments, wherein the nanoparticles comprise the cytoskeletal drug and the anti-angiogenic drug at a molar concentration ratio selected from the group consisting of 0.05:1, 0.1:1, 0.2:1, 0.3:1, 0.4:1, 0.5:1 0.6:1, 0.7:1, 0.8:1, 0.9:1, 1:1, 1:0.9, 1:0.8, 1:0.7, 1:0.6, 1:0.5, 1:0.4, 1:0.3, 1:0.2, 1:0.1, 1:0.05 or within a molar concentration ratio range bounded by any of these molar concentration ratios.

Embodiment 8. The composition of any of the foregoing embodiments, wherein the nanoparticles have an average effective diameter of <500 nm, and preferably have an average effective diameter of <400, 300, 200, 150, 100, or 50 nm, or have an average effective diameter within a range bounded by any of these values.

Embodiment 9. The composition of any of the foregoing embodiments, wherein the biodegradable nanoparticles comprise a biodegradable polymer.

Embodiment 10. The composition of any of the foregoing embodiments, wherein the biodegradable polymer of the biodegradable nanoparticles comprises polymerized carbohydrate monomers.

Embodiment 11. The composition of any of the foregoing embodiments, wherein the biodegradable nanoparticles comprise poly(lactic-co-glycolic acid) (PLGA).

Embodiment 12. The composition of any of the foregoing embodiments, wherein the wherein the biodegradable nanoparticles comprise PLGA 75:25 or PLGA 50:50.

Embodiment 13. The composition of any of the foregoing embodiments, wherein the surfactant comprises a water soluble polymer coupled to a hydrophobic molecule.

Embodiment 14. The composition of any of the foregoing embodiments, wherein the surfactant is polyethylene glycol coupled to a tocopherol, preferably D-a-tocopherol glycol 1000 succinate (i.e., TPGS).

Embodiment 15. The composition of any of the foregoing embodiments, wherein one or more of the components of the pharmaceutical composition inhibits the P-glycoprotein (P-gp) efflux transporter.

Embodiment 16. The composition of any of the foregoing embodiments, wherein the anti-angiogenic drug of the pharmaceutical composition (e.g., BIBF-1120) inhibits the P-glycoprotein (P-gp) efflux transporter.

Embodiments 17. The composition of any of the foregoing embodiment, wherein the surfactant of the pharmaceutical composition (e.g., TPGS) inhibits the P-glycoprotein (P-gp) efflux transporter.

Embodiment 18. The composition of any of the foregoing embodiments, wherein the pharmaceutical composition further comprises a T-cell stimulatory agent.

Embodiment 19. The composition of any of the foregoing embodiments, wherein the pharmaceutical composition further comprises an immune checkpoint inhibitor.

Embodiment 20. The composition of any of the foregoing embodiments, comprising: (a) PTX; (b) BIBF-1120; (c) nanoparticles, wherein the nanoparticles comprise PTX, BIBF-1120, or both of PTX and BIBF-1120; and (d) TPGS.

Embodiment 21. A method for treating a subject having a cancer characterized by loss-of-function of the p53 protein, the method comprising administering to the subject the pharmaceutical composition of any of embodiments 1-20.

Embodiment 22. The method of embodiment 21, wherein the cancer is selected from the group consisting of cancers of the adrenal gland, bladder, bone, bone marrow, brain, breast, cervix, gall bladder, ganglia, gastrointestinal tract, heart, kidney, liver, lung, muscle, ovary, pancreas, parathyroid, prostate, skin, testis, thymus, and uterus.

Embodiment 23. The method of embodiment 21 or 22, wherein the cancer is cancer of the uterus such as uterine serous carcinoma (USC).

Embodiment 24. A method for treating a subject having a cancer characterized by loss-of-function of the p53 protein, the method comprising: (a) administering to the subject a cytoskeletal drug that blocks progression of the cancer cells through mitosis, preferably PTX; and (b) administering to the subject an anti-angiogenic drug, preferably BIBF-1120.

Embodiment 25. The method of embodiment 24, wherein the cytoskeletal drug is administered substantially concurrently with the anti-angiogenic drug.

Embodiment 26. The method of embodiment 24 or 25, wherein the cancer is selected from the group consisting of cancers of the adrenal gland, bladder, bone, bone marrow, brain, breast, cervix, gall bladder, ganglia, gastrointestinal tract, heart, kidney, liver, lung, muscle, ovary, pancreas, parathyroid, prostate, skin, testis, thymus, and uterus.

Embodiment 27. The method of any of embodiments 24-26, wherein the cancer is cancer of the uterus such as uterine serous carcinoma (USC).

Embodiment 28. A method for treating a subject having a cancer susceptible to synthetic lethality, the method comprising administering to the subject a composition comprising nanoparticles and one or more cytotoxic and/or chemotherapeutic drugs that induce synthetic lethality.

Embodiment 29. The method of embodiment 28, wherein the cancer is characterized by loss-of-function of a tumor suppressor.

Embodiment 30. The method of embodiment 29, wherein the tumor suppressor is p53 or breast cancer protein 1 (BRCA1).

Embodiment 31. The method of any of embodiments 28-30, wherein the cancer is breast cancer.

Embodiment 32. The method of any of embodiments 28-31, wherein the cancer is breast cancer characterized by loss-of-function of BRCA1 and the one or more cytotoxic and/or chemotherapeutic drugs include an inhibitor of the poly ADP-ribose polymerase (PARP) 1 or 2.

Embodiment 34. The method of embodiment 28, wherein the one or more cytotoxic and/or chemotherapeutic drugs include an inhibitor of the p38 mitogen-activated protein kinase (MAPK) pathway.

Embodiment 35. A method for treating a subject having a cancer characterized by p53 deficiency or downregulation, the method comprising administering to the subject a pharmaceutical composition comprising nanoparticles, a cytoskeletal drug that block progression of cancers cells through mitosis, and an inhibitor of the p38 MAPK pathway, wherein less than about 100 mg of the inhibitor of the p38 MAPK pathway is administered to the subject.

Embodiment 36. The method of embodiment 34, wherein the cancer is characterized by a p53 mutation.

### EXAMPLES

The following examples are illustrative and should not be interpreted to limit the disclosed and claimed subject matter.

### Example 1

### Synthetically Lethal Nanoparticles for Treatment of Endothelial Cancer

Abstract

Uterine serous carcinoma (USC), one of the most aggressive types of endometrial cancer, is characterized by poor outcomes and mutations in the tumor suppressor p53. Our objective was to achieve synthetic lethality to paclitaxel (PTX), the frontline treatment for USC, in tumors with mutant p53 and enhance therapeutic efficacy using polymeric nanoparticles (NPs). First we identified the optimal NP formulation through a comprehensive analysis of release profiles, cellular uptake and cell viability. Not only were paclitaxel-loaded NPs (PTXp) superior to PTX in solution, but combination of PTXp with the antiangiogenic molecular inhibitor, BIBF-1120 (BIBF), promoted synthetic lethality specifically in USC with loss-of-function p53 mutation (LOF p53). In a xenograft model of USC, the combination therapy of BIBF+PTX, delivered as NPs, resulted in marked inhibition of tumor progression and extended survival. Together, our data provide compelling evidence for future studies of BIBF+PTX NPs as a therapeutic opportunity for LOF p53 cancers.

Introduction

Endometrial cancer (EC) arises from the epithelial cells lining the uterus and is considered the most prevalent gynecological malignancy in the USA¹. Over the last five years, both incidence and mortality for EC have substantially increased²⁻⁶, due in large part to the obesity epidemic. Importantly, EC is one of only two common cancers defying the general trend of improvement in incidence and mortality, with survival worse today than in the 1970s⁷. EC is classified into two major subtypes based on clinicopathological properties⁸. Type I EC is characterized by well differentiated cells of endometrioid origin and represents 80% of all cases⁹. This subtype is typically detected at an early stage and is associated with a favorable prognosis⁸. In contrast, type II EC includes mainly uterine serous carcinomas (USC), which comprise poorly differentiated and more aggressive cells and usually portend a poor prognosis⁹. Even though USC represents only 10% of all EC cases, it contributes to 39% of total EC deaths¹⁰. To date, the mainstay therapy for USC is multiple chemotherapies and/or radiotherapy, a standard that has been in place for over two decades^{11,12}. While numerous studies have explored the use of molecular inhibitors as monotherapies, these trials have generally failed to improve survival, suggesting that combinatorial therapies that rationally pair molecular inhibitors with standard chemotherapy may improve outcomes¹³.

Analysis of The Cancer Genome Atlas dataset for EC demonstrated that mutations in TP53 (the gene that encodes p53) predominate in USC, with mutations in 91% of cases as compared to only 11.4% of type I cases¹⁴. It is critically important to note that varying types of p53 mutant proteins exist. Mutations in TP53 are of three basic functional classes (1) truncating, frameshift or splice site loss of function (LOF) mutations that mainly result in protein instability and a p53-null state, (2) missense mutations that often result in gain of oncogenic function (GOF) via changes in DNA binding and protein:protein interactions, and (3) synonymous/silent mutations that are wild-type (WT) equivalent¹⁵.

As the guardian of the genome, p53 controls G1/S and G2/M cell cycle checkpoints to either allow cells to repair damaged DNA or induce apoptosis¹⁶. Activation of cell cycle checkpoints prevents progression into vulnerable phases of the cell cycle during treatment with chemotherapy. For example, paclitaxel (PTX), a widely used anticancer drug, kills dividing cells in mitosis (M) through stabilizing its mitotic-spindle microtubules¹⁷. Enforcing the G2/M checkpoint allows tumor cells to repair DNA before entering M, leading to chemoresistance¹⁸⁻²⁴. In addition to p53, emerging data suggest that p38MAPK can also maintain the G2/M checkpoint²⁵⁻²⁷. Therefore, in cells with LOF p53, p38 is activated as an alternative means to maintain the G2/M checkpoint²⁸.

Work from our group established that the combination of PTX with tyrosine kinase inhibitors (TKIs) induces synergistic cell death specifically in LOF p53 cancer cells due to abrogation of the alternative G2/M checkpoint^{29,30}. Cells arrest in M, cannot re-enter the cell cycle, and die due to mitotic catastrophe^{29,30}. This phenomenon is termed synthetic lethality, a historical genetic observation that in the presence of certain single gene mutations, blocking or mutating a second gene leads to cell death, though neither mutation alone has a phenotype^{31,32}. The concept of synthetic lethality has been explored in several clinical contexts, and the most successful to date is the use of PARP (poly (ADP-ribose) polymerase) inhibitors in tumors with mutations in BRCA³³⁻³⁶. With respect to the synergistic cell death by combination of PTX with TKIs, synthetic lethality means capitalizing on the presence of a p53 mutation to block the compensatory survival pathways activated as a result of the mutation. This approach is a novel application of synthetic lethality for p53 mutations given that the majority of studies have attempted to restore wild-type function³⁷. The advantage of this approach is that it adds a degree of cancer targeting as this combination will pose specific cytotoxicity only in cancer cells with a LOF p53 mutation, sparing the normal cells that do not carry the mutation.

Building on our previous work, herein we have developed an innovative approach to significantly enhance the efficacy of PTX+TKI combinatorial treatment for USC. First, we explored the use of a triple angiokinase molecular inhibitor BIBF-1120 (BIBF, also known as nintedanib) due to its inhibition of multiple tyrosine kinase receptors (vascular endothelial growth factor receptors, platelet derived growth factor receptors and fibroblast growth factor receptors³⁸) and induction of cell death when combined with PTX in USC cells³⁹. BIBF has been tested in several preclinical and clinical scenarios as a single agent or in combination with standard chemotherapy for a wide variety of cancers. Two large phase III trials in ovarian and non-small cell lung cancer demonstrated significantly improved progression-free survival when BIBF was combined either with paclitaxel-containing chemotherapy or with docatexel, which functions similarly to paclitaxel to arrest cells in mitosis^{40,41}. However, adverse effects, in particular gastrointestinal events, were increased in the groups that received nintedanib, indicating that additional strategies to improve the safety of the combinatorial strategy are necessary.

Second, we developed a polymeric nanoparticle (NP) delivery system to improve efficacy and maintain safety of the combinatorial strategy. NPs are well-established to 1) enhance dissolution, which overcomes the reported low water solubility of PTX and BIBF, 2) improve pharmacokinetics, 3) minimize side effects due to decreased off-target effects, and 4) passively target tumors through the enhanced permeability and retention effect (EPR)⁴². This is a phenomenon observed in solid tumors, where excessive angiogenic signals result in the formation of defective “leaky” tumor vasculature, through which NPs <200 nm in size can extravasate to the tumor microenvironment. In addition, the increased tumor mass leads to ineffective lymphatic drainage, which subsequently increases NP retention⁴³. Finally, we investigated the impact of varying NP formulations on major physicochemical properties of the prepared NPs, drug loading, cytotoxicity, cellular uptake and drug release.

Our findings demonstrate the superiority of the NP formulation over the soluble drug both in vitro and in vivo. In addition, the combination of PTX+BIBF in NPs exhibited significant reduction of tumor growth and equivalent safety in vivo when compared to either PTX in NPs or PTX in solution. Importantly, these findings were exclusive to USC cells with LOF p53. Together, these data provide the proof-of-concept evidence that synthetic lethality to PTX through combination with BIBF in NPs is an effective treatment strategy for USC and should be pursued as a personalized approach in patients with LOF p53 mutations.

Materials and Methods

Cell culture. Ishikawa H (Ishikawa, type I EC) and Hec50co EC cells (USC), a subline of Hec50 cells, were kindly provided by Dr. Erlio Gurpide (New York University)^{44,45}, and KLE cells (USC) were purchased from American Type Culture Collection (ATCC, Manassas, Va.). Hec50co cells stably expressing p53 R175H GOF (GOF Hec50co, USC) have been previously described²⁹. Ishikawa and Hec50co cells were cultured in Dulbecco's modified Eagle's medium (Gibco, Invitrogen, Waltham, Mass.) supplemented with 1% Pen/Strep (100 U/mL, Gibco) and 10% fetal bovine serum (FBS, Atlanta Biologicals, Lawrenceville, Ga.). KLE cells were cultured in RPMI-1640 medium (Gibco) supplemented with 1% Pen/Strep and 10% FBS. GOF Hec50co cells were cultured as Hec50co cells with the addition of 0.8 mg/mL G418 to main stable p53 R175H expression (Gibco). All cells were maintained in a humidified incubator (Sanyo Scientific Autoflow, IR direct heat CO₂ incubator) at 37° C. under 5% CO₂ flow. All cell lines were authenticated by CODIS marker testing, and were mycoplasma-free as determined by MycoAlert mycoplasma detection kit (Lonza, Rockland, Me.).

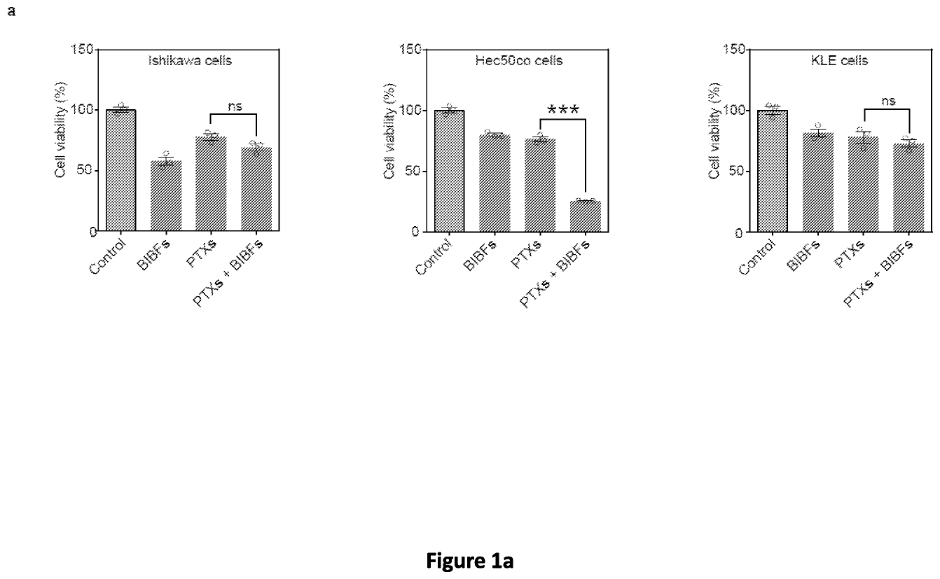

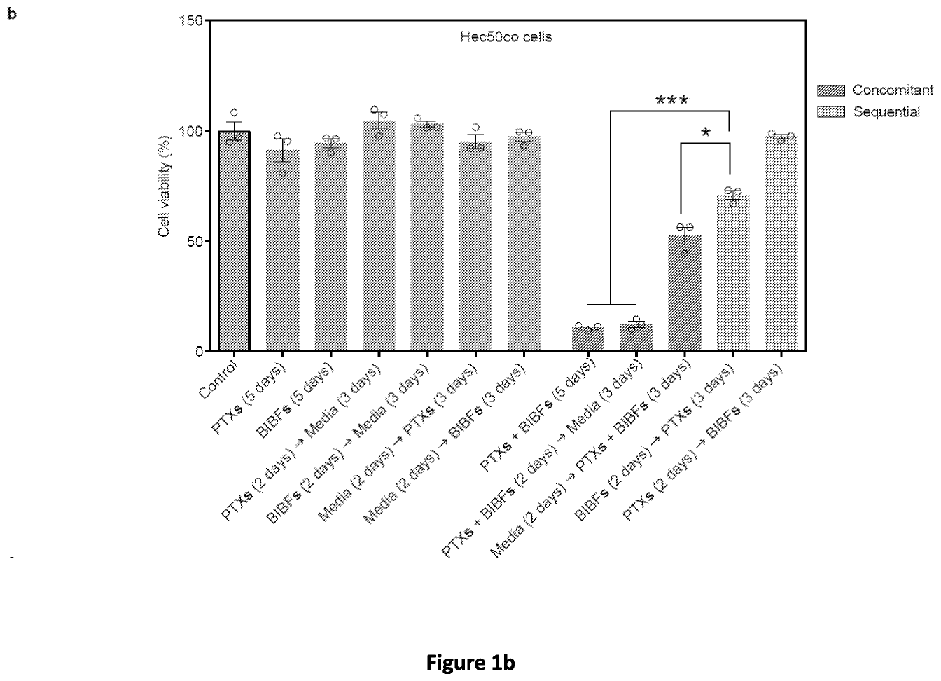

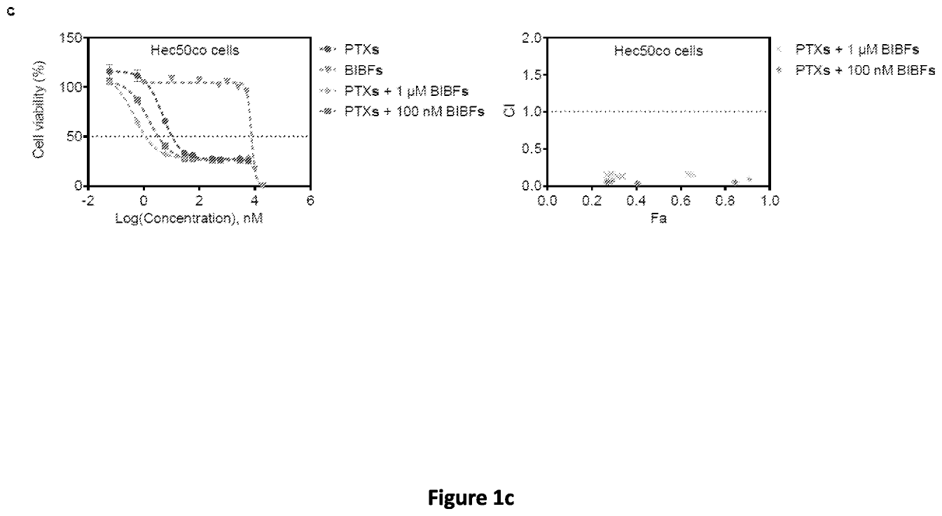

Cell viability assay. Two days (48 h) prior to adding the treatments, Ishikawa, Hec50co and GOF Hec50co cells were plated at a density of 10³ cells/well, while the slower growing KLE cells were plated at 0.5×10⁴cells/well, in 96 well plates. Treatments were added in a volume of 50 μL/well followed by the addition of 150 μL/well of fresh media. The untreated control group was incubated with 200 μL/well of fresh media. Three days (72 h) later, all of the 96 well plate contents were aspirated, replaced by 100 μL of fresh media and 20 μL of MTS tetrazolium compound in each well (CellTiter 96 Aqueous One Solution Reagent, Promega Corporation, Madison, Wisc.). Cells were incubated with MTS reagent at 37° C. with 5% CO₂ for 1-4 h. The absorbance was recorded at 490 nm using a Spectra Max plus 384 Microplate Spectrophotometer (Molecular Devices, Sunnyvale, Calif.). Relative cell viability values were expressed as the percentage of the absorbance from wells containing treated cells compared to the control wells containing untreated cells. Viability of control wells were set to be equal to 100%. The contribution of plain media to the absorbance value was taken into consideration through measuring the absorbance of a cell free well that contained only media and MTS reagent, and subtracting this absorbance value from those in the treated wells. For experiments where both concomitant and sequential administration of PTX and BIBF were evaluated (FIG. 1b), Hec50co cells were seeded at 10³ cells/well for 48 h. The first treatment was added for another 48 h, washed away and then the second treatment was added for an extra 72 h, followed by assessment of viability. The untreated control group was incubated with fresh media for 5 days. Synergy between PTXs and BIBFs was evaluated in Hec50co cells through the establishment of dose response curves of PTXs, BIBFs or the combination using varied concentrations of PTXs and either 1 μM BIBFs or 100 nM BIBFs. As stated above, cells were plated in 96 well plates at a seeding density of 10³ cells/well for 48 h. Different treatments were then added for an additional 72 h, and cytotoxicity was evaluated using MTS cell proliferation assay. Combination index (CI) values were calculated by utilizing the dose response curve data in CompuSyn software (ComboSyn Inc., Paramus, N.J.): a CI<1 indicates synergy.

NP Fabrication and Characterization

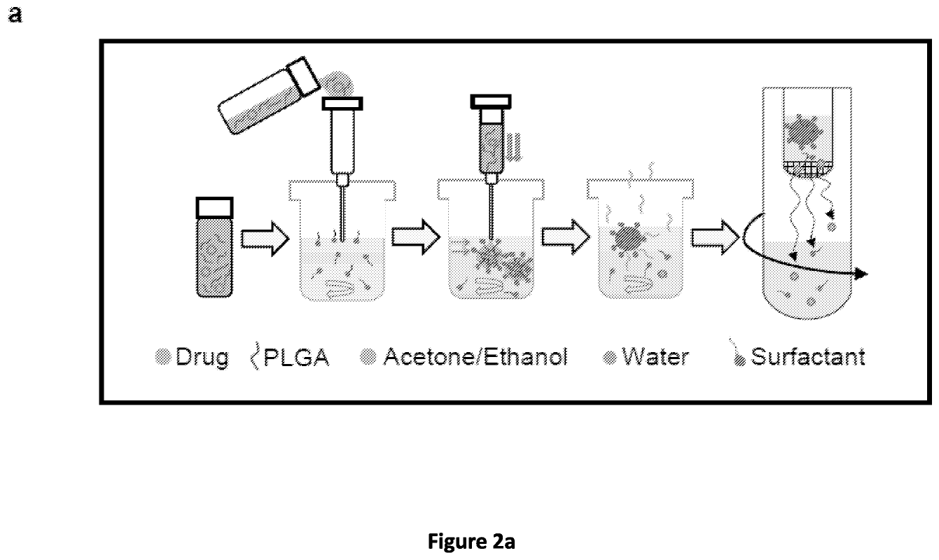

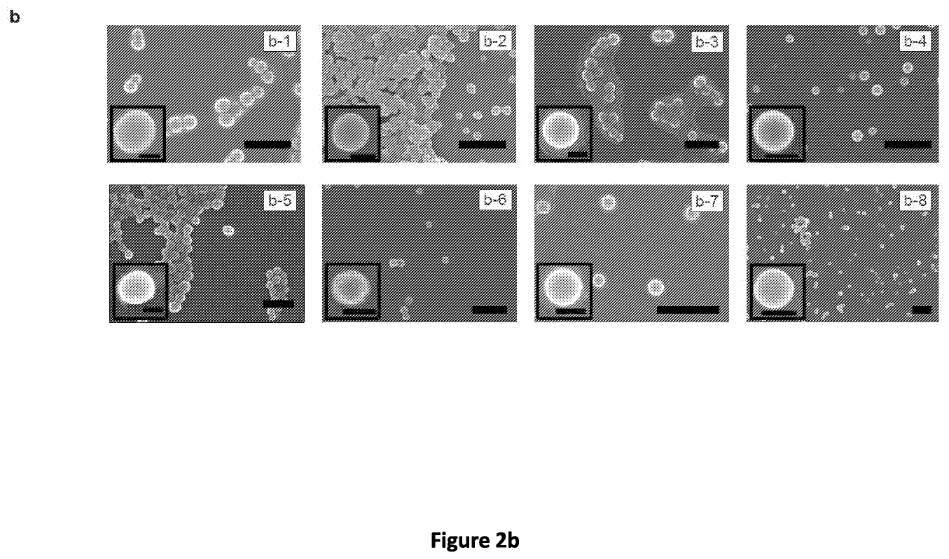

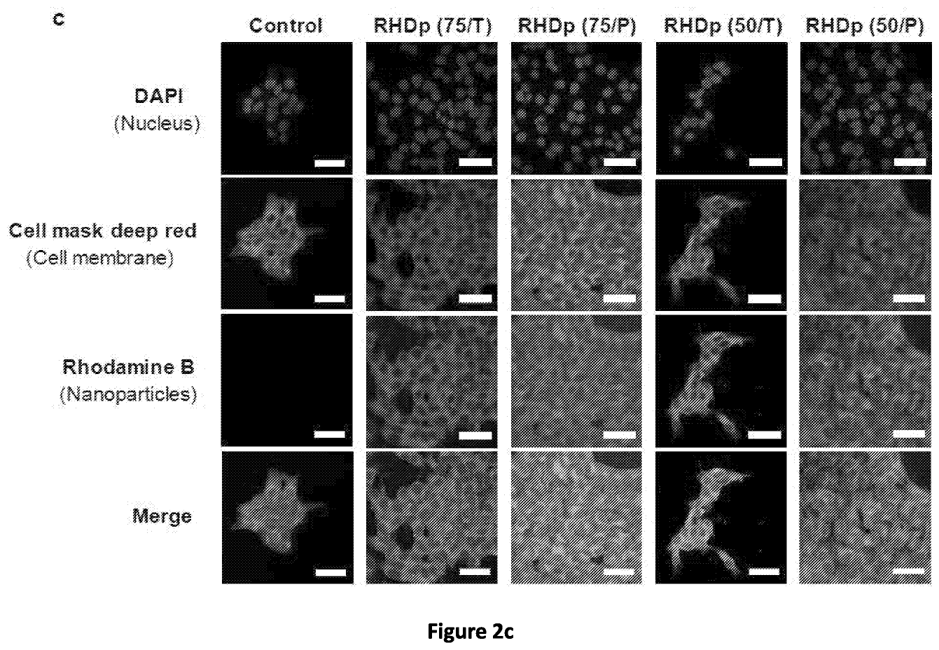

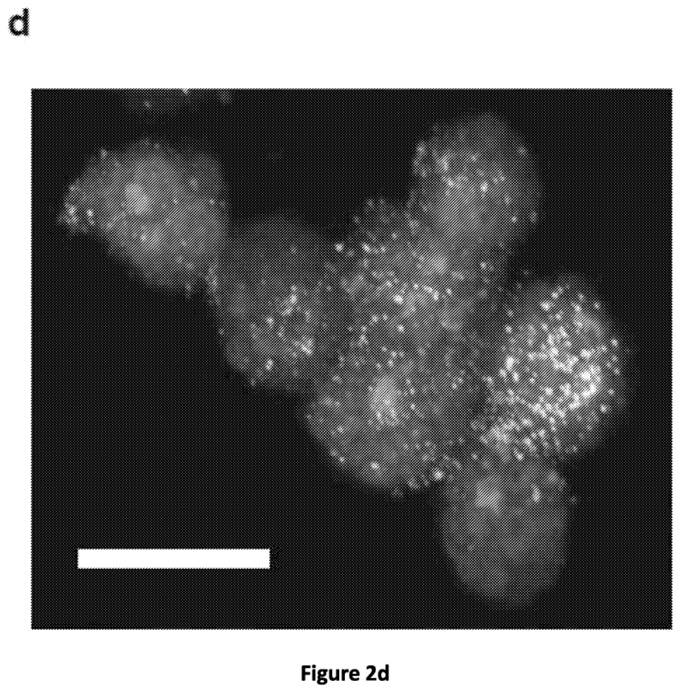

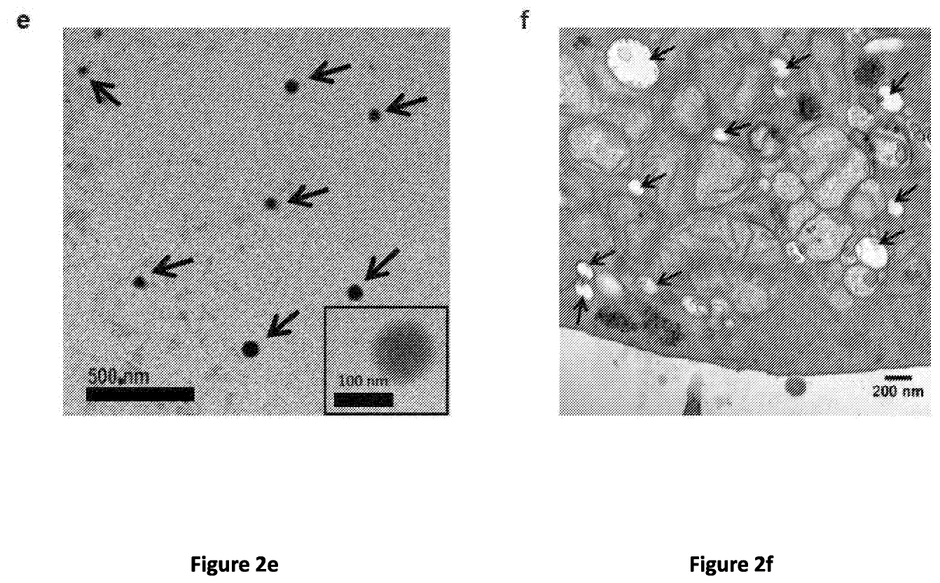

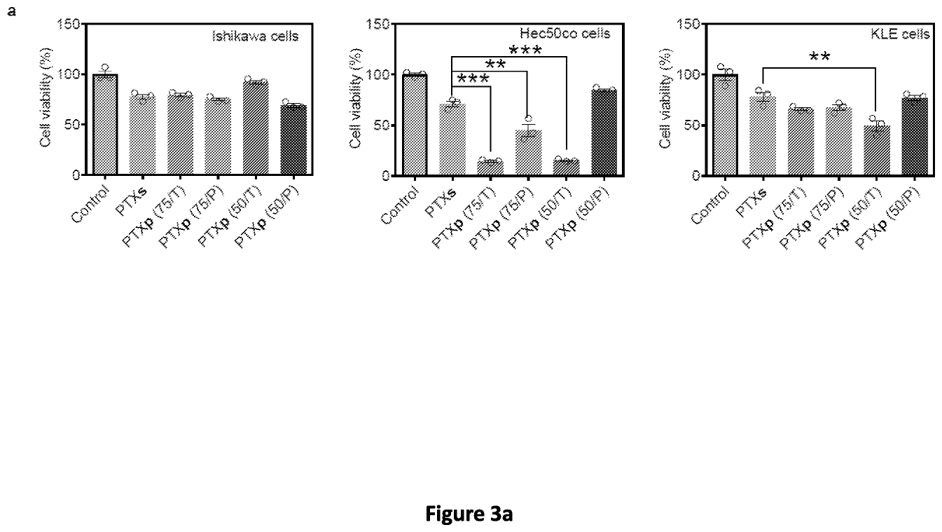

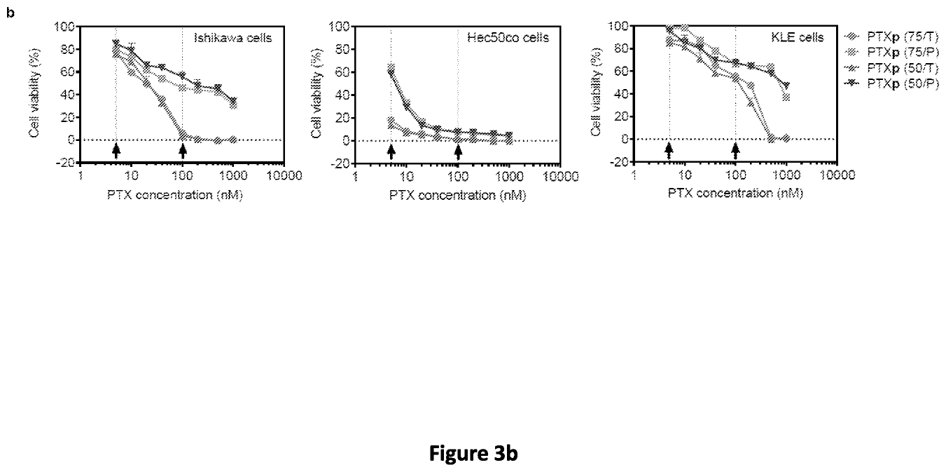

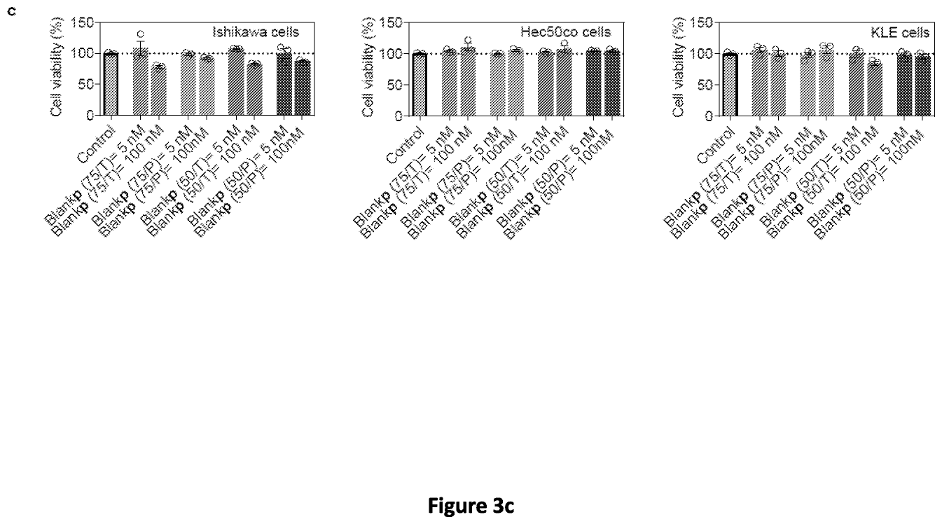

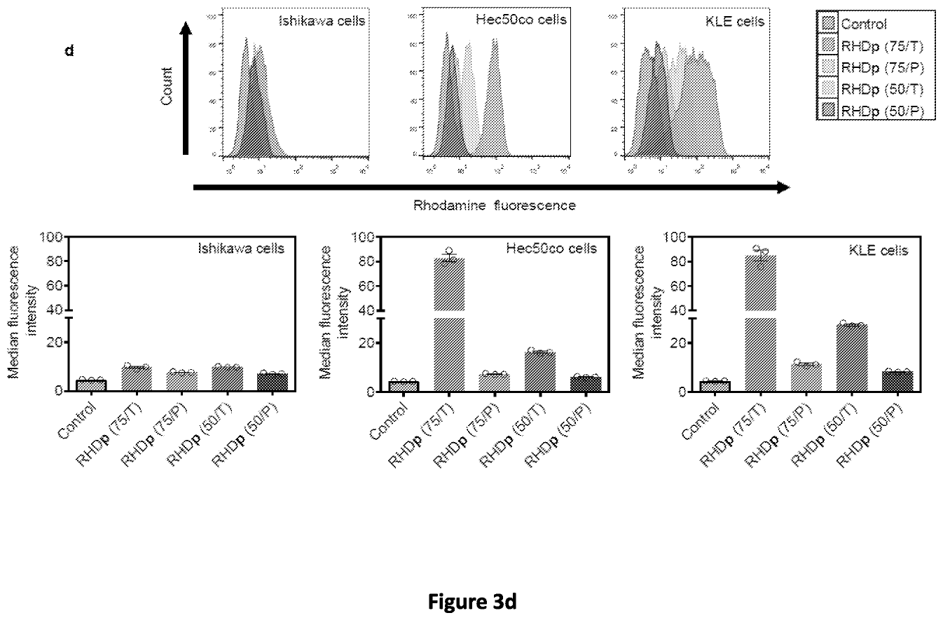

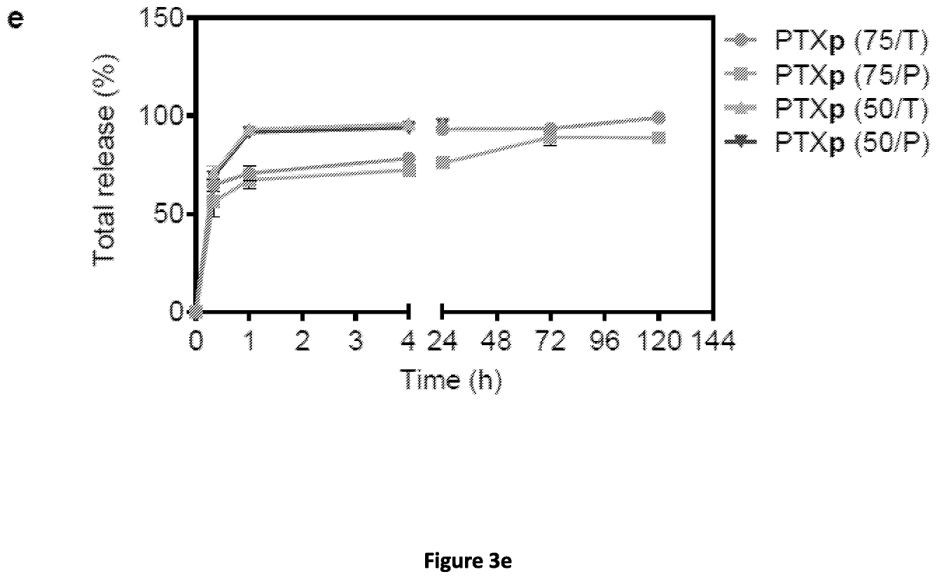

NP fabrication. NPs were prepared using the nanoprecipitation method as diagrammed in (FIG. 2A). Briefly, 5 mg of drug (paclitaxel (PTX) (LC Laboratories, Woburn, MA) or BIBF 1120 (BIBF) (Selleck Chemicals, Houston, Tex.)) and 100 mg of polymer (poly [lactic-co-glycolic acid] (PLGA, 75:25, molecular weight, Mw, of 68 kDa, inherent viscosity of 0.59 dL/g, Durect Corporation, Pelham, Ala.)) or PLGA (50:50, Mw of 24-38 kDa, inherent viscosity of 0.32-0.44 dL/g, Resomer RG 503H, Boehringer Ingelheim KG, Germany)) were dissolved in 4.25 mL acetone (Fisher Scientific, Waltham, Mass.), and 0.75 mL 97% ethanol (Sigma-Aldrich, St. Louis, Mo., USA). This organic phase was added to a 10 mL syringe, with a needle size of G26, placed such that the tip was submerged just below the surface of stirred 50 mL of aqueous solution containing 0.1% w/v surfactant (Poly(vinyl alcohol) (PVA, Mw 8-9 kDa, 80% hydrolyzed, Sigma) or D-α-tocopherol polyethylene glycol 1000 succinate (TPGS, Sigma)) in a 150 mL beaker. The formed suspension was left on the stirrer for 45 min and then the rest of the organic solvent was evaporated under reduced pressure of 40 mbar using Laborota 4000 rotary evaporator (Heidolph, Schwabach, Germany) for 4 h. NPs were then washed with nanopure water and collected using Amicon ultra-15 centrifugal filter units (Mw cut off=100 kDa, EMD Millipore, Billerica, Mass.) at 500×g for 15 min 4 times using an Eppendorf centrifuge 5804 R (Eppendorf, Westbury, N.Y.). NPs were freshly prepared before each experiment. For (FIG. 3a&b) PTXp (75/T) and PTXp (75/P) were prepared on the first day, stored overnight at 4° C., and then PTXp (50/T) and PTXp (50/P) were prepared on the second day, when all the treatments were initiated. This staggered preparation of NPs was necessary as the preparation of each batch takes ˜6-7 h.

Estimation of drug loading and encapsulation efficiency. NPs were dissolved in acetonitrile and drug content was estimated through HPLC-UV for PTX and HPLC-MS for BIBF. PTX content in the NPs was quantified using HPLC-UV (2690 Alliance separation module coupled with 2487 dual λ absorbance detector, Waters, Milford, Mass.). Reverse phase 5 μm C-18 column, 100 A°, 4.5×250 mm (Waters) was utilized in the assay and isocratic elution with a mobile phase of acetonitrile (Fisher Scientific): water (60:40, v/v) at a flow rate of 1 mL/min was used. The detection wavelength was set at 227 nm and the injection volume was 100 μL. BIBF content was determined using HPLC-Mass (Shimadzu Model 2010A liquid chromatograph and mass spectrometer, Shimadzu, Columbia, Md.) using a LC-10AD VP Solvent Delivery system. Synergi 4 μm Polar-RP column, 80 A°, 2×150 mm (Phenomenex Inc, Torrance, Calif.) was used. Isocratic elution was utilized with a mobile phase composed of water+0.1% formic acid (Fisher Scientific): acetonitrile+0.1% formic acid (50:50, v/v), at a flow rate of 0.2 mL/min. Electrospray ionization was used, m/z ratio of 540.5 was utilized, and 25 μL was injected.

Drug loadings and encapsulation efficiencies were calculated from the following formulas.

$$Drug loading ( \frac{μ g of drug}{mg of NPs} ) = \frac{Amount of PTX in NPs ( μ g )}{Total weight of NPs ( mg )}$$

$$Encapsulation efficiency ( \% ) = \frac{Amount of PTX in NPs ( mg )}{Initial amount of PTX ( mg )} \times 100$$

NP size and zeta potential determination. NPs suspension of 0.05 mg/mL was prepared in water. Size and zeta potential were then measured using a Zetasizer Nano ZS particle analyzer (Malvern Instrument Ltd., Westborough, Mass.). NPs size was measured at 173° backscatter detection in disposable polystyrene cuvettes. Zeta potential was measured in a zeta potential folded capillary cell.

Microscopic Evaluation of NPs

Electron microscopy. Surface morphology of prepared NPs was examined using scanning electron microscopy (SEM). Briefly, NPs suspension of 0.05 mg/ml was added onto a silicon wafer mounted on an aluminum SEM stubs using double stick carbon tape. The suspension was allowed to air dry for 24 h. They were then coated with gold and palladium by an argon beam K550 sputter coater (Emitech Ltd., Kent, England). Images were captured using the Hitachi S-4800 scanning electron microscope (Hitachi High-Technologies, Ontario, Canada), operated at 3 kV accelerating voltage.

Confocal laser scanning microscopy. Qualitative cell uptake studies of the prepared NPs were carried out using confocal microscopy. Briefly, rhodamine B (RHD, Sigma) loaded PLGA NPs (RHDp) were prepared by the nanoprecipitation method as described previously, except that the drug was substituted by an equivalent amount of RHD. The RHD content in RHDp was calculated by dissolving the NPs in DMSO and then comparing RHD fluorescence to a constructed calibration curve (data not shown). RHD fluorescence was measured at λex 540 nm and λem 625 nm using a SpectraMax M5 multi-mode microplate reader (Molecular Devices, Sunnyvale, Calif.). Hec50co cells were plated at density of 10⁴ cells in a clear, flat-bottom, 4-chambered glass slides with a lid (Lab-Tek, Nunc, Rochester, N.Y.), and incubated for 48 h (37° C., 5% CO₂). RHD loaded PLGA NPs (RHDp) containing 0.01 μg RHD were then added to each chamber, leaving untreated cells as a control, and incubated with the cells for 4 h (cells in Z stacked confocal image were incubated for 24 h with RHDp (75/T), FIG. 2d). Media was removed and cells were washed twice with Hank's balanced salt solution (Gibco). Cell membranes were stained by adding 0.5 mL of prewarmed cell mask deep red plasma membrane stain solution (Invitrogen) at 5 μg/mL in each chamber, incubated for 5 min at 37° C., washed and replaced by 0.5 ml of fresh media for another 5 min. Media was then aspirated, washed twice with phosphate buffer saline (PBS, Gibco). Then 0.5 mL of 4% paraformaldehyde (Hatfield, Pa., USA) fixative solution was added and incubated for 10 min at 37° C. The specimen was mounted with Vectashield Hardset medium containing DAPI (Vector laboratories, Burlingame, Calif.) for staining the nuclei. The cellular fluorescence was observed using confocal laser scanning microscopy (Carl Zeiss 710, Germany) equipped with Zen 2009 imaging software. The images were processed using Image J open access software, version 1.47 (National Institutes of Health, Md.).

Transmission electron microscopy. The size and shape of PTX loaded NPs (PTXp) prepared from PLGA (75:25) and TPGS surfactant (PTXp (75/T)) were also measured by JEOL JEM-1230 transmission electron microscope (TEM) equipped with a Gatan UltraScan 1000 2k×2k CCD acquisition system ((JEOL USA Inc., Peabody, Mass.). 10 μL of NPs suspension (0.05 mg/mL) was added for 30 secs on a carbon coated, glow discharged 400-mesh TEM copper grid by Auto 306 (BOC Edwards, Crawley, United Kingdom) that was pre-coated with a Formvar 0.5% solution in ethylene dichloride film (Electron Microscopy Sciences, Hatfield, Pa.). Whatman filter paper was then used to remove any excess liquid and the grid was air dried. The TEM images were processed using Image J.

Cellular uptake of PTXp (75/T) was further confirmed through TEM. HEC50co cells were seeded at 10⁶ cells in a 100 mm petri dish for 24 h. PTXp (75/T) at concentration equivalent to 5 nM PTX were then incubated with the cells for another 24 h. Cells were then fixed with 2.5% glutaraldehyde (Electron Microscopy Science, EMS, Hatfield, Pa.) in 0.1 M sodium cacodylate buffer (EMS), pH 7.4, for 30 min, rinsed twice with 0.1 M cacodylate buffer, pH 7.4, for 4 min each. 1% osmium tetroxide (EMS) was then added for 30 min to increase electron density and improve fixation efficiency. Fixed cells were then washed twice with distilled water and stained with 2.5% uranyl acetate (EMS) for 5 min. Dehydration of the sample was performed gradually using 25%, 50%, 75%, 95% ethanol, each for 4 min, and finally twice with 100% ethanol for 5 min each. Dehydrated samples were infiltrated with ethanol: Epon (Ted Pella, Inc., Redding, Calif.) mixture (1:1) for 30 min, and then embedded in Epon at 70° C. for 8 h. Thin nanometer sections of 60-80nm were cut using Leica EM UC6 Ultramicrotome MZ6 (Reichert-Jung, Reichert, Depew, N.Y.), finally these sections were mounted on Formvar-coated 400-mesh TEM copper grid, counter stained with 5% uranyl acetate and Reynold's lead citrate (80 mM lead nitrate (Sigma) in 164 mM sodium citrate buffer (RPI, Mt. Prospect, Ill.)). Sample was then imaged, and then processed using Image J.

Quantitative uptake of NPs by flow cytometry. Quantitative cell uptake was carried out using FACScan flow cytometer (Becton Dickinson, Franklin Lakes, N.J.). Ishikawa. Hec50co and GOF Hec50co cells were plated at density of 10⁵ cells, while KLE cells were plated at density of 0.5×10⁶ cells in 12 well plates. One day (24 h) later, equivalent amount of RHDp containing 0.01 μg RHD was added to each well in serum free media, and untreated cells were used as control. 6 h later, cells were washed with PBS twice, trypsinized, quenched with serum containing media, centrifuged at 230×g for 5 min, resuspended in 300 μL of fresh media and kept on ice untill analysis was performed. Serum free media was used to accelerate the uptake process of these particles and thus differences in the magnitude of NPs uptake would be easily detected over a short period of incubation.

PTX in vitro release. PTX release studies from different formulations were performed by adding PTXp equivalent to 1 μg PTX in 1 mL of 1% v/v aqueous tween 80 solution (Fisher Scientific) in 1.5 mL amber microcentrifuge tube. Samples were incubated at 37° C. in a horizontal shaker at 300 rpm. At each time point, 3 tubes were centrifuged at 20817×g for 20 min at 5° C., the supernatant was discarded, and the amount of drug remaining in particles was estimated by dissolving the pellet in acetonitrile, vortexed for 10 min, and finally analyzed using HPLC. The total amount released at each time point was calculated by subtracting the amount of PTX remaining in the pellet from the original amount of PTX added to each tube.

Cell cycle analysis by flow cytometry. Cells were plated in 100 mm dishes with an equal number of cells in each dish and treated with either 1 μM of soluble BIBF (BIBFs), 40 nM of PTXp (75/T), or combination of both for 24 h. Cells were fixed in 70% ethanol. After washing with PBS, cells were incubated in Krishan's solution (3.8 mM sodium citrate (Fisher Scientific), 0.014 mM propidium iodide (AnaSpec, Fermont, Calif.), 1% NP-40 (Sigma) and 2.0 mg/mL RNase A (Fisher Scientific)) for 30 minutes at 37° C. and analyzed by FacScan flow cytometer as previously described³⁰. The data were subjected to further analysis by CellQuest software version 3.3, which was used to generate DNA histograms indicating the fractions of the cell population in the sub-G1, GO-G1, S or G2/M phase of the cell cycle.

Western blot analysis. As previously described³⁰, cells were plated in 100 mm dishes and were allowed to grow for 24 h prior to adding the treatment. Cells were treated with either 1 μM of BIBFs, 40 nM of PTXp (75/T), or combination of both for 24 h, and then cells were harvested, lysed with extraction buffer (1% Triton X-100 (Sigma), 10 mM Tris-HCl (Sigma) pH 7.4, 5 mM EDTA (Sigma), 50 mM NaCl (Sigma), 50 mM NaF (Fisher Scientific), 20 μg/ml aprotinin (Fisher Scientific), 1 mM PMSF (Fisher Scientific), and 2 mM Na3VO4 (Fisher Scientific)), and subjected to three freeze/thaw cycles as previously described³⁰. Equal amounts of protein (determined by the method of Bradford, BioRad, Hercules, Calif.) were subjected to SDS-PAGE (BioRad) followed by transfer to nitrocellulose membranes (BioScience, San Jose, Calif.). Membranes were probed with primary antibodies against total CDC2 (catalogue no. 9112), phospho-cdc2 Tyr 15 (catalogue no. 9111), CDC25C (catalogue no. 4688) and phospho-histone H3 Ser10 (catalogue no. 3377, Cell Signaling Technology, Danvers, Mass.) followed by incubation with corresponding horseradish peroxidase-conjugated secondary antibody (catalogue no. 7074, Cell Signaling Technology). The signal was visualized by chemiluminescence using ECL Western blotting detection reagents (Pierce, Fisher Scientific).

BIBFs effect on NPs uptake using flow cytometry. The effect of BIBFs on the uptake of different RHDp against Hec50co cells and GOF Hec50co cells was tested. Both cell lines were plated at a seeding density of 10⁵ cell/well in 12 well plate, and then the experiment was carried out as mentioned in section [00125]) with two exceptions: a) RHDp uptake was evaluated in the presence or absence of 1 μM BIBFs, b) the experiment was carried out in serum containing media to mimic the in vivo conditions.

In vivo efficacy studies using mouse xenograft model of LOF p53 USC. Female athymic NCI-nu/nu mice (Charles River, Wilmington, MA) at the age of 6-8 weeks were challenged subcutaneously with 2×10⁶ Hec50co cells in the right flank after isoflurane anesthesia. Once the tumor volumes reached 50 mm³, mice were randomized into four groups, and were then treated with either saline (naïve), 5 mg/kg PTXs in 10% (v/v) Tween 80 solution, 5 mg/kg PTXp (75/T), or the combination therapy of 5 mg/kg PTXp (75/T) and 5 mg/kg BIBFp (75/T). Mice were 5 per group, except the group that received the combination therapy were 7. Treatments were administered IV through retro-orbital injections in the venous sinus on days 18, 25, and 32. The tumor diameters and height were measured using digital caliper. The tumor volumes were calculated from the following formula:

$$Tumor volume ( {mm}^{3} ) = \frac{π}{6} \times {D}_{1} \times {D}_{2} \times H$$

Where D₁ is the first tumor diameter (mm), D₂ is the second tumor diameter (mm), and H is the tumor height.

Mice weights were monitored during the experiment and mice were euthanized once the tumor diameter exceeded 2 cm or tumor height exceeded 1 cm. Sample sizes for this experiment were estimated based on preliminary data in order to have 80% power to detect significant differences between groups. All animal experiments were not blinded, and were carried out in accordance with guidelines and regulations approved by the University of Iowa Institutional Animal Care and Use Committee.

Histological evaluation of the NPs safety. Once tumor-challenged mice were euthanized, heart, lung, liver, spleen and kidney were harvested, fixed in 10% neutral buffered formalin (RPI), and then embedded in paraffin (EM-400, Surgipath, Leica Biosystems Inc., Buffalo Grove, Ill). Sections of 5μm were prepared, stained with H & E (Leica Biosystems Inc.), and imaged using Olympus BX61 microscope (Olympus, Center Valley, Pa.). Finally, images were processed using Cell Sens software (Olympus).

Estimation of PTX intra-tumoral drug concentration. Female athymic NCI-nu/nu mice at the age of 6-8 weeks were challenged subcutaneously with 2×10⁶ Hec50co cells in the right flank after isoflurane anesthesia. Once the tumor volumes reached ˜500 mm³, mice were IV (retro-orbital injection) treated with either 5 mg/kg PTXs or 5 mg/kg PTXp (75/T). Tumors were collected 1, 4 and 12 h post injection, and PTX concentration within the tumor was quantified using a validated LC-MS/MS method (see supplementary information for additional experimental details).

Statistical analysis. All in vitro experiments were repeated at least twice (n=3). Data are expressed as mean±SEM. Statistical analysis was performed using GraphPad prism software for Windows version 6.07 (GraphPad Software, Inc., San Diego, Calif.). One-way analysis of variance (ANOVA) followed by Tukey post hoc test was used to compare between groups. In vivo tumor progression curves were analyzed utilizing the non-parametric Kruskal-Wallis test. Kaplan-Meier survival curves were analyzed using the Log-rank test with the Bonferroni post hoc test. Assessment of statistical differences between groups in the estimation of PTX intra-tumoral drug concentration experiment was carried out using an unpaired two-tailed t-test. Differences were considered significant at p<0.05.

Results and Discussion

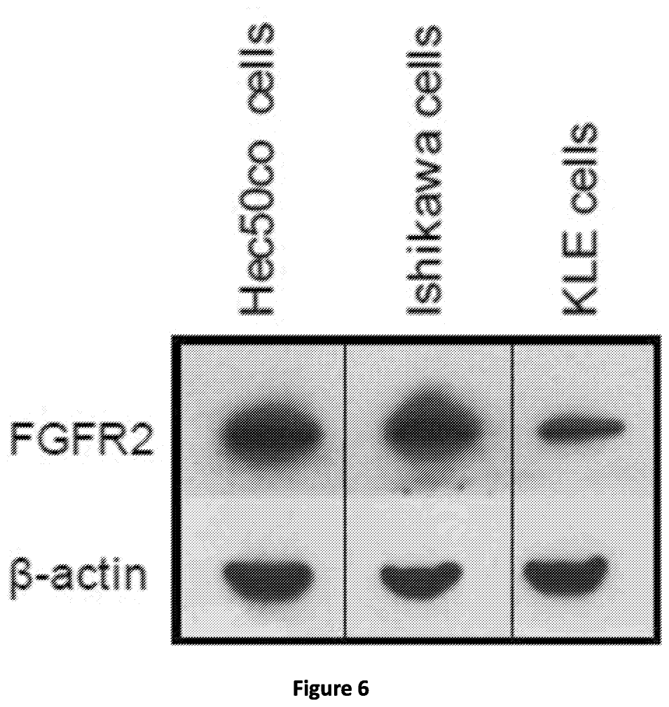

Synthetic lethality to combination therapy of soluble BIBF and PTX in LOF p53 cells. Our first goal was to investigate the involvement of p53 mutational status. on the sensitivity of EC cell lines to the combination therapy of BIBF and PTX. Three different EC cell lines bearing different p53 mutations were utilized in the study: Ishikawa cells (WT p53), Hec50co cells (LOF p53 mutation that results in a p53-null status) and KLE cells (GOF p53 due to R175H mutation). Since endometrial cancer cells have been reported to harbor FGFR2 activating mutations, we screened all cells for FGFR2 expression and mutational status. These cells all express FGFR2 (FIG. 6), and the sequence is wild-type as determined by sequencing mutational hotspot regions in the third immunoglobulin domain and the transmembrane domain. Cells were incubated with either soluble PTX (denoted as “PTXs”) or soluble BIBF (denoted as “BIBFs”) or a combination of both drugs concomitantly. Analysis of cell viability revealed that only Hec50co cells with LOF p53 were sensitive to the combination therapy (FIG. 1a), with a three-fold decrease in viability compared to PTXs or BIBFs as single agents (p<0.001). These data substantiate the dependence of the combination of PTX and BIBF on LOF p53 status.

In the combinatorial setting, most anti-cancer drugs are administered simultaneously, though it has been suggested that sequential or time-staggered administration may improve therapeutic efficacy⁴⁶. Based on the ability of BIBF to abrogate the G2/M checkpoint and induce mitotic catastrophe when combined with PTX in LOF p53 cells²⁹, we hypothesized that pretreatment of Hec50co cells with BIBFs prior to PTXs will enhance cytotoxicity as compared to the concomitant treatment protocol. However, concomitant administration of both drugs (red bars) was superior to sequential administration (orange bars, FIG. 1b). When cells were concomitantly treated with both agents on days 1-5, days 1-2, or days 3-5 of culture, cell viability was reduced to 10.9, 12.4 and 52.5% of control levels, respectively. Thus, synthetic lethality does not require molecular priming with BIBF. All subsequent experiments used concomitant drug administration, which also negated the need to generate NP formulations with different release profiles. Calculation of the combination index demonstrated pronounced synergy between paclitaxel and BIBF at concentration as low as 100 nM (FIG. 1c).

TABLE 1
Characterization of Blankp and PTXp prepared using different PLGA
grades and different surfactants as well as BIBFp (75/T).

| | | | | | Drug |
|---|---|---|---|---|---|
| | Particle | | Zeta | Encapsulation | loading |
| Formula | size | | potential | efficiency | (μg drug/mg |
| abbreviation | (d · nm) | PDI | (mV) | (%) | nanoparticles) |
| Blankp (75/T) | 136.7 ± 2.2 | 0.06 ± 0.04 | −47.9 ± 2.0 | — | — |
| Blankp (75/P) | 173.6 ± 3.8 | 0.05 ± 0.04 | −40.2 ± 4.0 | — | — |
| Blankp (50/T) | 138.0 ± 4.3 | 0.06 ± 0.03 | −48.4 ± 5.7 | — | — |
| Blankp (50/P) | 167.3 ± 3.1 | 0.04 ± 0.01 | −34.2 ± 0.6 | — | — |
| PTXp (75/T) | 140.7 ± 4.0 | 0.18 ± 0.10 | −47.2 ± 3.2 | 56.4 ± 3.7 | 47.0 ± 3.1 |
| PTXp (75/P) | 163.1 ± 4.9 | 0.11 ± 0.05 | −40.1 ± 5.8 | 31.6 ± 3.2 | 26.3 ± 2.7 |
| PTXp (50/T) | 143.1 ± 7.2 | 0.09 ± 0.02 | −52.2 ± 5.5 | 38.9 ± 2.9 | 32.4 ± 2.4 |
| PTXp (50/P) | 147.8 ± 10.5 | 0.07 ± 0.06 | −41.7 ± 6.2 | 25.0 ± 0.9 | 20.8 ± 0.8 |
| BIBFp (75/T) | 109.5 ± 15.1 | 0.09 ± 0.01 | −42.8 ± 5.4 | 49.7 ± 8.3 | 41.4 ± 6.9 |

Data are presented as mean ± SD (n = 3).
75 = PLGA (75:25), 50 = PLGA (50:50), T = TPGS, P = PVA

Preparation and characterization of PTX-loaded NPs. Our next objective was to design a delivery system that would enhance the cytotoxic effect of these drugs both in vitro and in vivo, and overcome the drawbacks of administering soluble drugs in vivo. Polymeric NPs were chosen as our delivery system due to their ability to offer superior drug stability, higher accumulation in the tumor, enhanced tumor regression, and lower systemic side effects as compared to injecting soluble drug⁴⁷. Specifically, biocompatible poly [lactic-co-glycolic acid] (PLGA) NPs were prepared utilizing 1) two different PLGA polymers of different monomer ratios and different molecular weights (Mw, (75:25, Mw=67 kDa and 50:50, Mw=24-38 kDa), and 2) two different surfactants: polyvinyl alcohol (PVA), and D-α-tocopherol polyethylene glycol 1000 succinate (TPGS). PVA is the most commonly used surfactant in NP fabrication based on its superior surfactant characteristics⁴⁸. TPGS is a promising surfactant that has been recently used in NP fabrication, with a distinct ability to inhibit P-glycoprotein (P-gp) efflux transporter in addition to its activity as a surface active agent⁴⁹.

NPs were prepared using a nanoprecipitation method (FIG. 2a), a simple technique capable of producing small nanometer scale particles with narrow size distribution to more easily predict the in vivo behavior of the NPs. PTX was the drug used in this study. We first assessed the impact of the varying formulation parameters on major physicochemical properties of NPs: shape, size, and zeta potential, as well as drug loading, cytotoxicity, cellular uptake, and drug release.

A NP size <200nm potentiates passive targeting to the tumor via the EPR effect and would be expected to show superior cytotoxicity in vivo as compared to the soluble drug⁴³. As shown in Table 1, all of the prepared NPs were less than 175 nm in diameter, with a narrow size distribution and a net negative charge. When TPGS was used as the surfactant, the NPs exhibited smaller hydrodynamic diameters as compared to PVA as the surfactant. PTX loading into NPs (denoted as “PTXp”) did not significantly alter size or zeta potential as compared to blank NPs (“Blankp”). PTXp prepared using PLGA (75:25) and TPGS as a surfactant (“PTXp (75/T)”), exhibited 1.8-times higher encapsulation efficiency (EE) and drug loading (DL) compared to when PVA was used as a surfactant (“PTXp (75/P)”). The same trend was achieved when PLGA (50:50) was used to prepare the NPs, and thus a 1.6-fold higher EE and DL was achieved with TPGS (“PTXp (50/T)”), compared to PVA (“PTXp (50/P)”). These findings are consistent with previously published work⁵⁰. The higher EE and DL that accompanied the use of TPGS are likely due to the increased affinity of PTX for the hydrophobic vitamin E portion of the surfactant that was embedded in the NPs matrix⁵⁰. Higher EE and DL were associated with the use of PLGA (75:25) when compared to PLGA (50:50) when the same surfactant was utilized, which is likely due to the higher lactic acid content and subsequently superior hydrophobic characteristics that PLGA (75:25) attained when compared to the PLGA (50:50). For example, PTXp (75/T) showed a 1.4 fold higher EE and DL when compared to PTXp (50/T).

SEM images demonstrate that all of the prepared formulations were spherical with smooth surfaces, and loading PTX in the NPs (FIG. 2b, panels 1-4) did not affect the integrity or surface morphology as compared to Blankp (FIG. 2b, panels 5-8). There was no significant difference in size between TPGS and PVA prepared NPs (e.g. FIG. 2b, panels 1&2). It should be noted that there is a discrepancy in NP size estimated using zeta sizer (Table 1) or based on SEM images, which is likely due to the fact that the hydrodynamic diameter, as measured by the zeta sizer, tends to overestimate the size of NPs with hydrophilic surfaces. PVA has a higher hydrophilic lipophilic balance value of 18 as compared to 13.2 for TPGS⁵¹, and thus the higher hydrophilic characteristics and higher hydrodynamic diameter values with PVA relative to TPGS was expected.

To evaluate the cellular uptake of the prepared NPs, fluorophore rhodamine B (RHD) was loaded in the NPs (termed “RHDp”) instead of PTX. Confocal microscopy images demonstrate NP uptake by Hec50co cells within 4 h of incubation (FIG. 2c), which was confirmed by a Z-stacked confocal image of RHDp (75/T) incubated with Hec50co cells for 24 h (FIG. 2d).

TEM images of PTXp (75/T) verified the SEM data, as the particles were spherical and less than 175 nm in size (FIG. 2e). Finally, to validate that the confocal microscopy images were detecting NPs and not simply free RHD that had leached out of the NPs, PTXp (75/T) was incubated with Hec50co cells for 24 h, and then cells were processed and imaged using TEM. The TEM image confirmed the cellular uptake of the NPs (black arrows) and their anticipated cytoplasmic distribution (FIG. 2f).

Identification of NP formulation with superior in vitro cytotoxicity, uptake, and drug release. Having established that NPs with various formulations are internalized by EC cells, we next studied the differences in cytotoxicity as compared to PTXs. In Ishikawa (WT p53) and KLE cells (GOF p53), NPs showed comparable decreases in cell viability relative to PTXs, with the exception of PTXp (50/T) which exhibited a 36% enhancement in cytotoxicity in KLE cells as compared to PTXs (p <0.01, FIG. 3a). Interestingly, in Hec50co cells, all NP formulations except PTXp (50/P) promoted a significant decrease in cell viability, with the most notable effects with the TPGS-emulsified NPs (PTXp (75/T) and PTXp (50/T), p<0.001, FIG. 3a). Dose response curves using varied concentrations of the different PTXp formulations validated these findings (FIG. 3b). We also confirmed that the cytotoxicity is not due to the NP formulation by repeating viability studies using Blankp at amounts equivalent to the 5 and 100 nM PTXp doses (FIG. 3c). Together, these data demonstrate that PTXp are specific for LOF p53 cells and have greater therapeutic efficacy in vitro as compared to PTX in solution.

Our data also demonstrate that the surfactant TPGS is far superior to PVA in LOF p53 Hec50co cells. Given that TPGS has been reported to inhibit drug efflux transporters⁵², we hypothesized that the improved cell killing with PTXp (75/T) and PTXp (50/T) is due to the increased intracellular retention of PTX. To test this, we performed flow cytometry cell uptake experiments utilizing RHDp Like PTX⁵³, RHD is a substrate for the P-gp efflux transporter⁵⁴ and can serve as a surrogate for PTX intracellular behavior, with the additional advantage of being detectable using flow cytometry.

The three EC cell lines were incubated with RHDp in serum-free media to maximize uptake of the NPs⁵⁵ and thus facilitate detection of any small difference between uptake of different NP formulations. In all three cell lines, we detected an increase in RHD accumulation with the use of TPGS as surfactant when compared to the use of PVA (FIG. 3d). Interestingly, in Hec50co cells, the median fluorescence intensity of RHDp (75/T) was almost 11.4-times higher than that of RHDp (75/P), and RHDp (50/T) showed a 2.7-times higher fluorescence intensity when compared to RHDp (50/P), supporting our hypothesis that TPGS increases intracellular drug accumulation.

In addition, RHDp (75/T) exhibited a 5.1-fold increase in fluorescence intensity when compared to RHDp (50/T), indicative of a difference in uptake. This increase in uptake could be related to the fact that PLGA (72:25) is more hydrophobic than PLGA (50:50)⁵⁶. Indeed, both Ishikawa and KLE exhibited the same trend in NP uptake, though the magnitude of NP uptake was much higher in KLE cells (similar to Hec50co). Based on these data, we surmise that Hec50co and KLE cells have higher expression of efflux transporter(s) relative to Ishikawa cells. Although KLE cells had higher accumulation of RHDp (75/T) (FIG. 3d), this was not reflected in higher cytotoxicity (FIG. 3a). This could be related to the fact that KLE cells are insensitive to PTX when compared to Hec50co cells (Supplementary Fig. S2). These experiments suggest that the PTXp (75/T) formulation has the best uptake profile.

We also investigated the drug release profile for the different NP formulations and found that NPs with a higher Mw PLGA (75:25) had a slower release profile for PTX compared to the lower Mw (50:50) polymer (FIG. 3e). These data are consistent with previous results that the higher the Mw of the polymer, the longer the polymer chain, the more hydrophobic the polymer, and subsequently the slower the degradation and the release of the loaded drug⁵⁷. In addition, polymers with a higher lactic acid content, like PLGA (75:25), have a higher hydrophobicity and consequently slower interaction with water and slower degradation and drug release⁵⁷.

At the 24 h time point, PTX release was slightly higher from PTXp (75/T) than PTXp (75/P). The apparent accelerated drug release with TPGS could be due to two possibilities. First, PTX has a high affinity for the vitamin E moiety of TPGS, which is expected to be oriented on the surface of the NPs. This would increase the availability of PTX for release as compared to PVA-emulsified NPs⁵⁰. Another possible explanation is based on the reported faster release of docetaxel from TPGS-emulsified PLGA NPs. This study found that TPGS forms pores at the NPs surface, and thus increases the exposed surface area to the release media ⁵⁸.

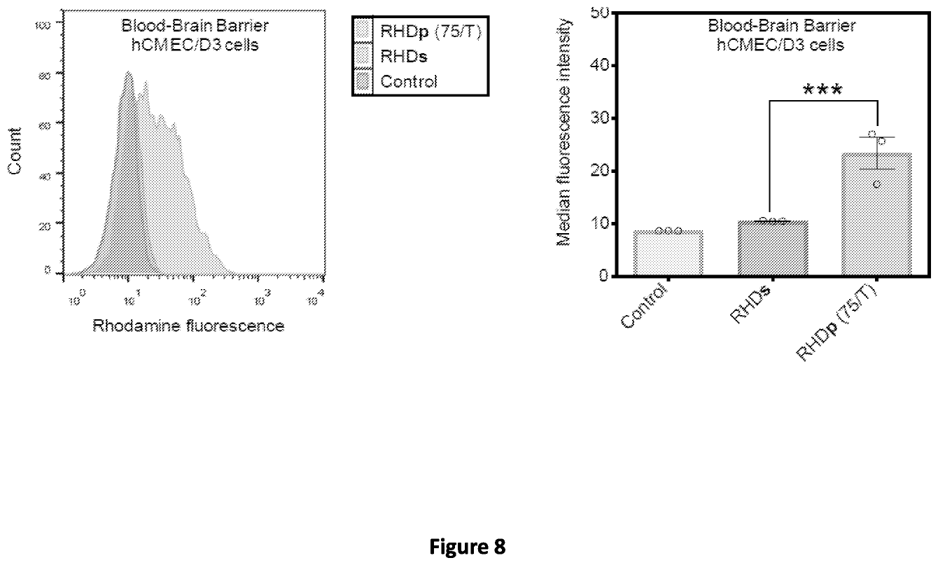

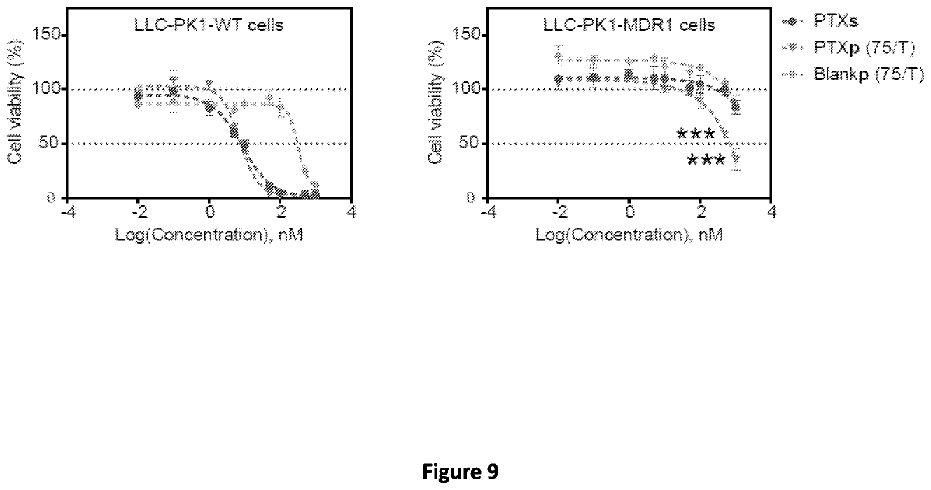

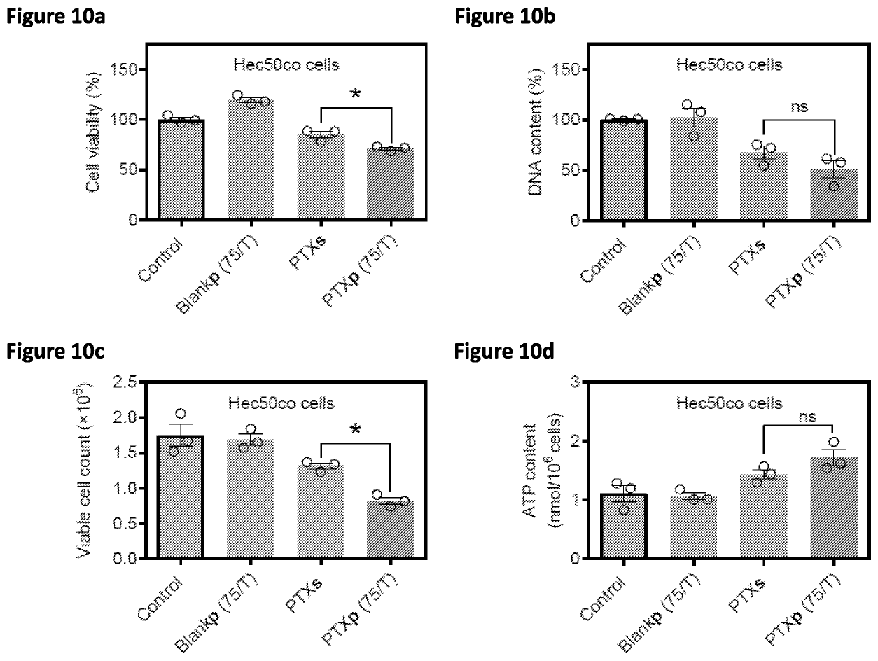

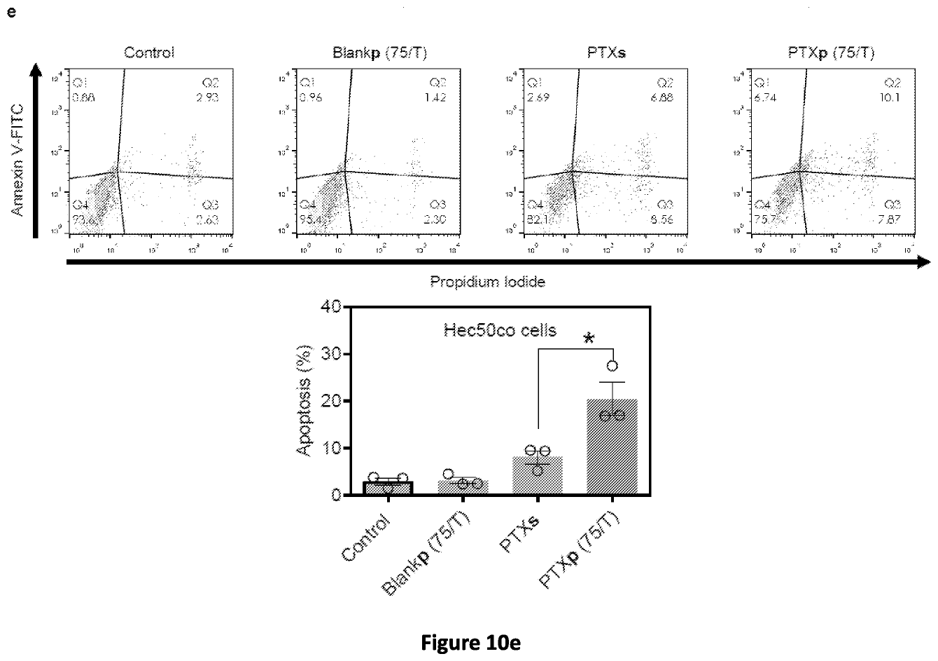

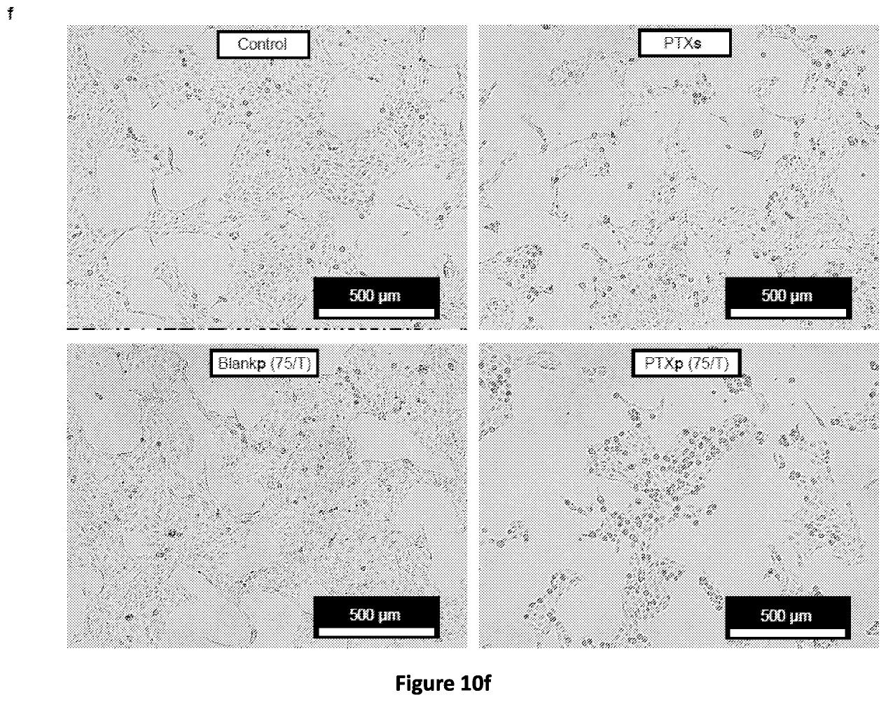

Based on these results, PTXp (75/T) was selected as the optimum formulation. In separate experiments using a well-established efflux transporter blood-brain barrier model, we confirmed that the RHDp (75/T) formulation has a robust uptake profile as compared to RHDs (FIG. 8). We also established that PTXp (75/T) significantly decreased cell viability in a cell model of paclitaxel resistance (FIG. 9). Marked cell death in cells treated with PTXp (75/T) was confirmed using multiple methods, including analysis of viable cell numbers, DNA, ATP content, and apoptosis (FIG. 10). Blank NPs had no effect on any of these parameters.

PTX-loaded NPs induce synthetic lethality when combined with BIBF in USC cells with LOF but not GOF p53. PTXp (75/T) was used in subsequent combinatorial experiments with BIBF. Specifically, we combined BIBFs with the selected formulation, PTXp (75/T), to confirm that NP formulations of PTX do not abrogate synthetic lethal activity, and define the molecular mechanisms underlying the synergy between BIBF and PTX in LOF p53 USC cells.

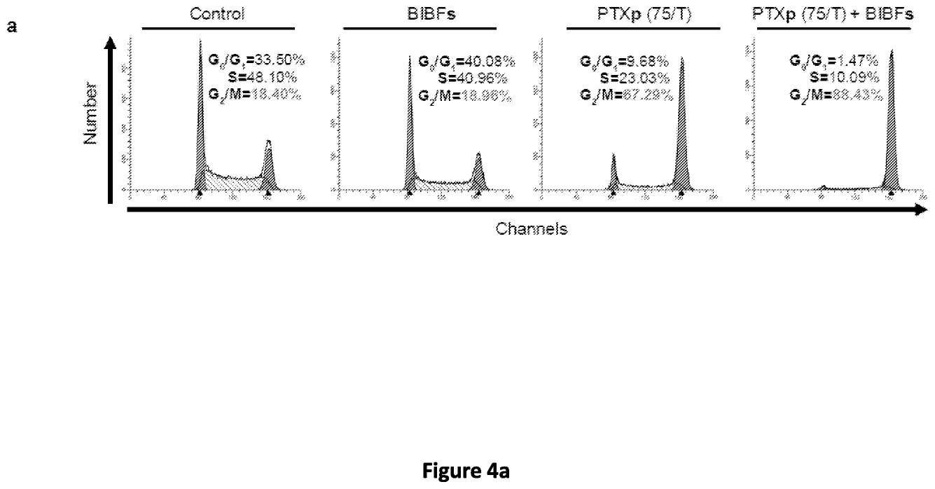

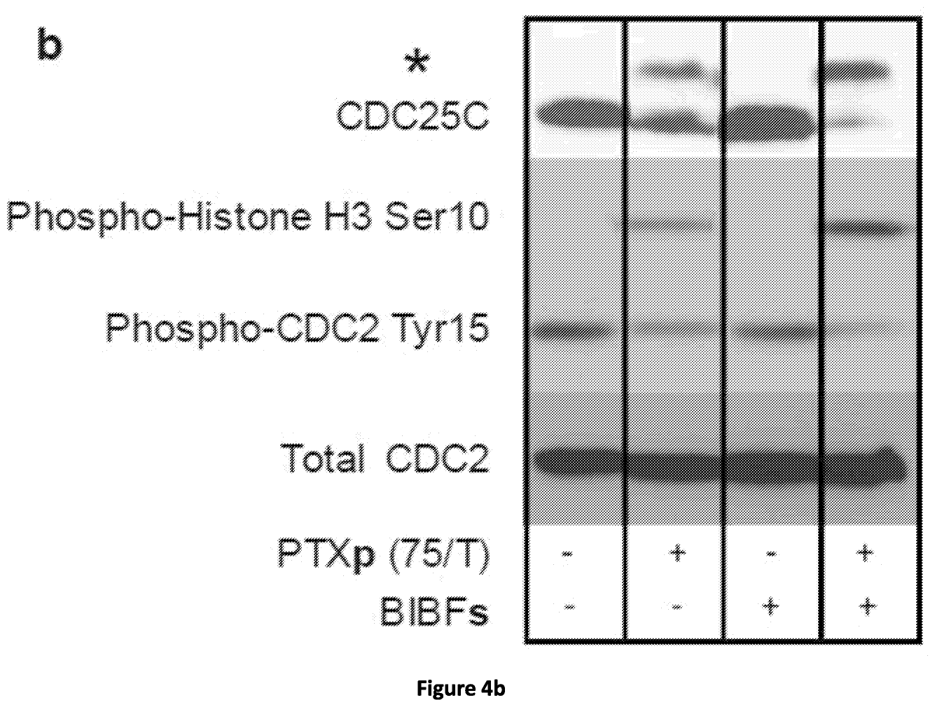

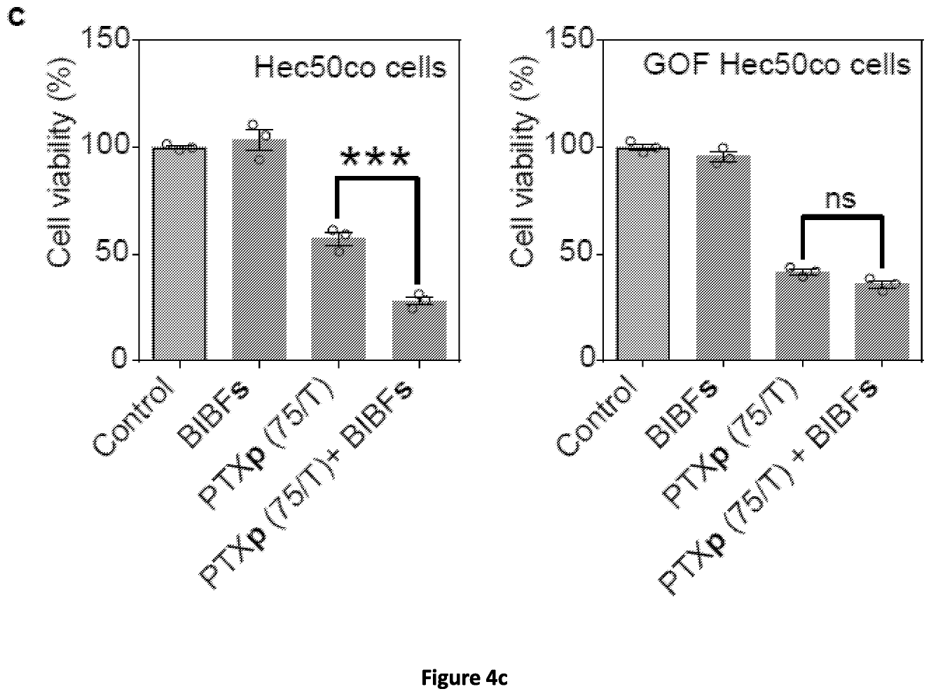

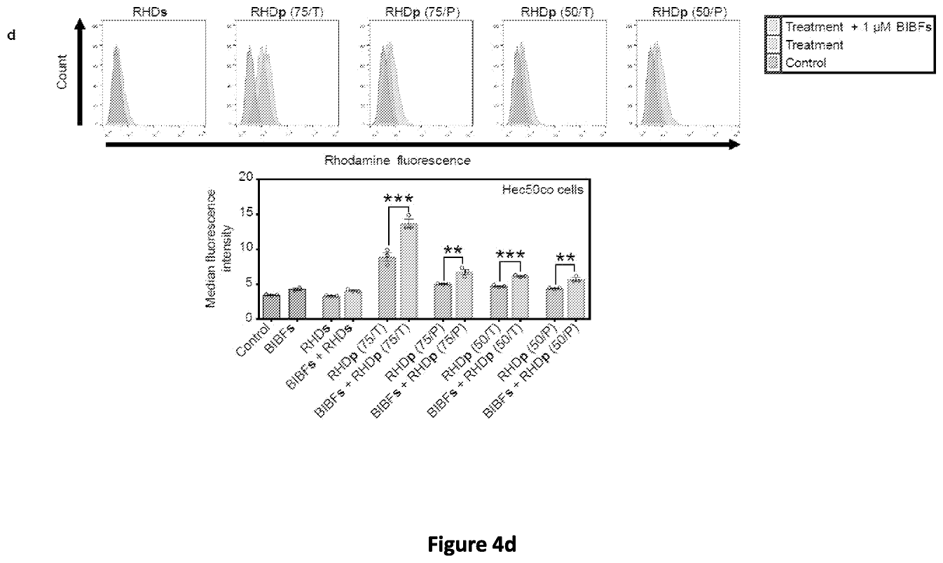

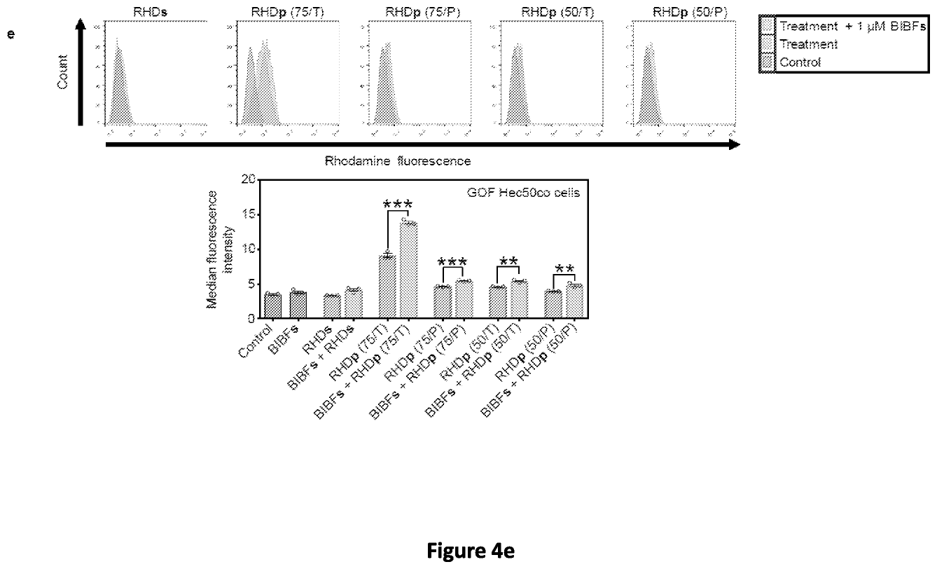

We first explored the effect of the combinatorial treatment on the cell cycle progression in Hec50co cells (FIG. 4a). Consistent with previously published data using drugs in solution²⁹, PTXp (75/T) promoted accumulation of a large proportion of cells in G2/M compared to control treatment (67.3%, FIG. 4a). However, the addition of BIBFs to PTXp (75/T) resulted in nearly all cells accumulating at G2/M. In addition to molecular effects on the G2/M checkpoint, BIBF has been reported to inhibit the P-gp efflux transporter⁵⁹, which would prevent PTX efflux and increase its intracellular concentration and subsequently its effect.

We next evaluated key G2/M regulators to determine if the combinatorial treatment produced the anticipated abrogation of the G2/M checkpoint. Phosphorylation of the kinase CDC2 at Tyr 15 maintains the G2/M checkpoint, whereas dephosphorylation of CDC2 by the phosphatase CDC25C results in entry into M phase. CDC25C is also maintained in an inhibited state through phosphorylation at Ser 216, which is mediated by multiple kinases including p38MAPK. Therefore, abrogation of the G2/M checkpoint requires dephosphorylation of CDC25C at Ser 216 and phosphorylation at 12 different sites (indicated by a slower migrating band) and decreased phosphorylation of CDC2 at Tyr 15. Dual treatment with BIBFs and PTXp (75/T) resulted in decreased CDC2 Tyr 15 and increased CDC25C activation, as noted by a slower migrating band (denoted with a star, FIG. 4b). Finally, we also detected increased phosphorylation of histone H3 at Ser 10, which is a marker for mitosis.

We also established combination treatment of BIBFs and PTXp (75/T) induces synthetic lethality in Hec50co cells. Consistent with the data in FIG. 1, dual treatment produced the most profound decrease in cell viability as compared to either drug alone (FIG. 4c, left panel). We further examined the involvement of p53 status in the mechanism of synthetic lethality by overexpressing p53 GOF mutant in p53-null Hec50co cells (“GOF Hec50co cells”). In contrast to parental Hec50co cells, the GOF Hec50co cells did not show any additional increase in cytotoxicity from the combinatorial treatment over PTXp (75/T) alone (FIG. 4c), demonstrating the requirement for LOF p53 status for the synthetic lethal effect.

The difference in toxicities due to p53 status could be attributed to one of two possibilities. 1) In the absence of p53, cells rely on the p38 pathway to maintain the G2/M checkpoint^{39,60}, and treatment with a TKI like BIBF in combination with PTX will reduce p38 activation^{39,61}. In the GOF p53 cells, cells have evolved an additional mechanism to maintain p38 phosphorylation through increased expression of the upstream kinase MKK3³⁹. Hence, treatment with a TKI is not sufficient to abrogate the G2/M checkpoint as in GOF p53 cells. 2) The change in p53 status is accompanied by a change in the expression of efflux transporter(s) and thus corresponding differences in PTX intracellular accumulation. Given that BIBF has been reported to inhibit P-gp⁵⁹, this change in drug efflux would alter the cytotoxicity of the combination therapy. The relationship between P-gp expression and p53 status has been controversial: Thottassery et al showed that LOF p53 is associated with upregulation of P-gp⁶², whereas Angelis et al showed that there is no correlation⁶³. In addition, a positive correlation between GOF p53 and P-gp overexpression has been reported by Sampath and colleagues™.

We therefore performed a cell uptake experiment to examine if BIBFs increases the accumulation of RHD, a P-gp substrate, and if there is a difference in the magnitude of RHD accumulation. Complete media was used in this experiment to mimic in vivo conditions. Although RHDp (75/T) was already chosen as the optimum formulation, we also tested different formulations. BIBFs significantly enhanced the accumulation of RHD inside both parental and GOF Hec50co cell lines and for all tested formulations (FIG. 4d&e). Consistent with data in FIG. 3, RHDp (75/T) showed the highest accumulation in both cell lines, which supports its choice as the optimum formulation. Moreover, addition of BIBFs to RHDp (75/T) resulted in a 1.5-fold increase in fluorescence intensity in both cell lines, indicating that the magnitude of enhancing the accumulation of the P-gp substrate is the same in both cell lines. These data support the interpretation that the synthetic lethality observed in (FIG. 4c) is likely due to interference with the G2/M checkpoint in LOF p53 cells and not due to variable expression in efflux transporters in cells with different p53 mutational status.

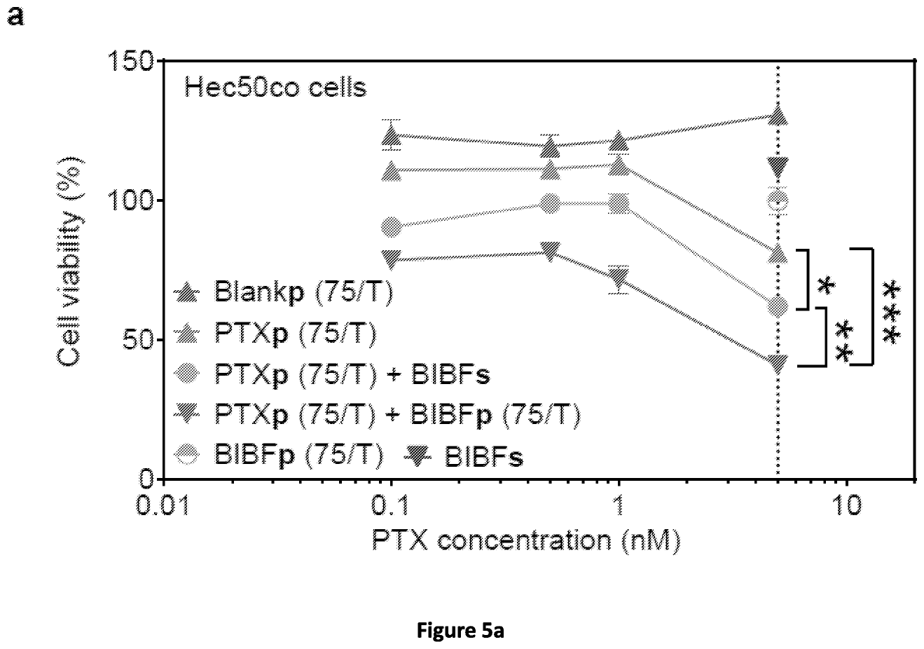

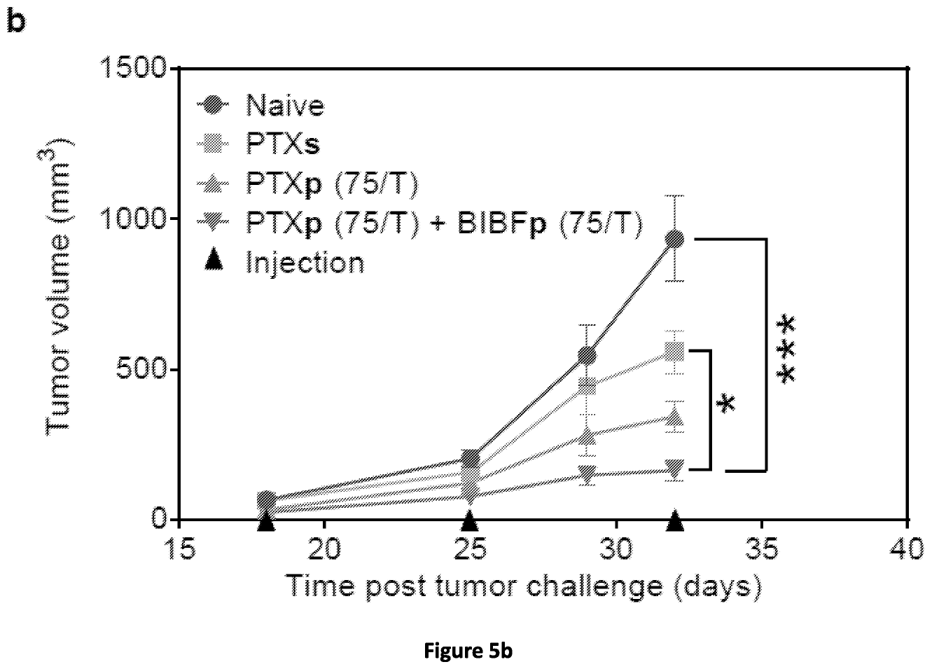

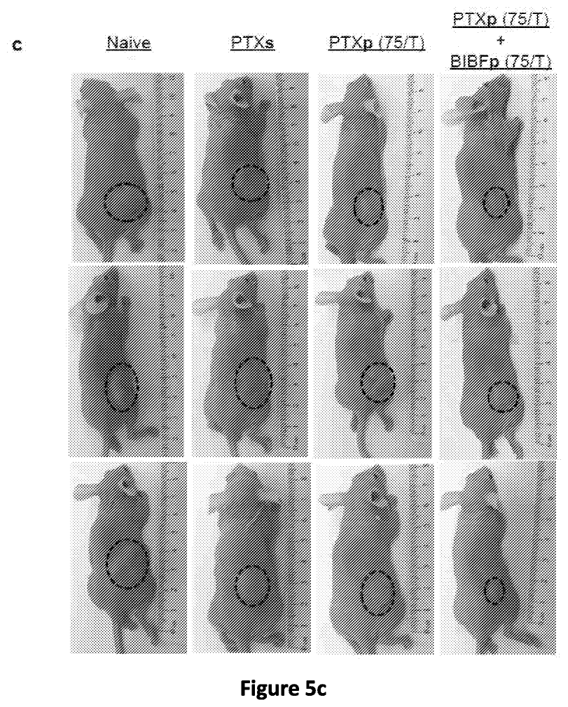

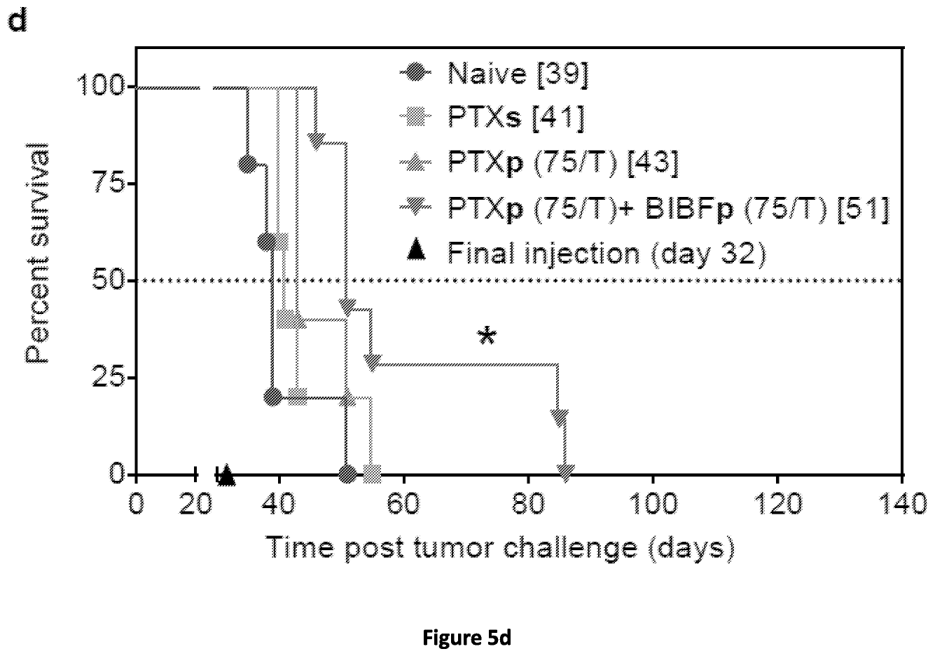

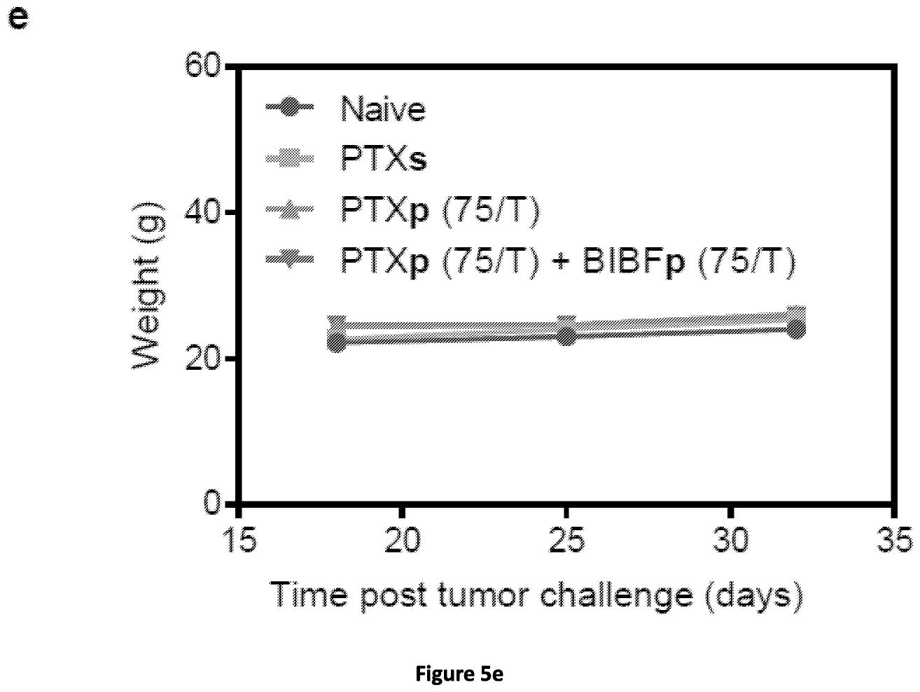

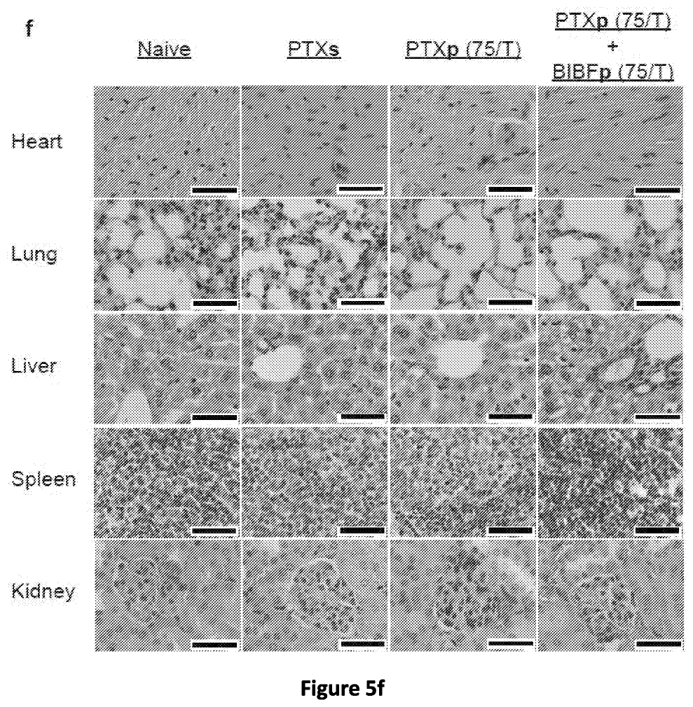

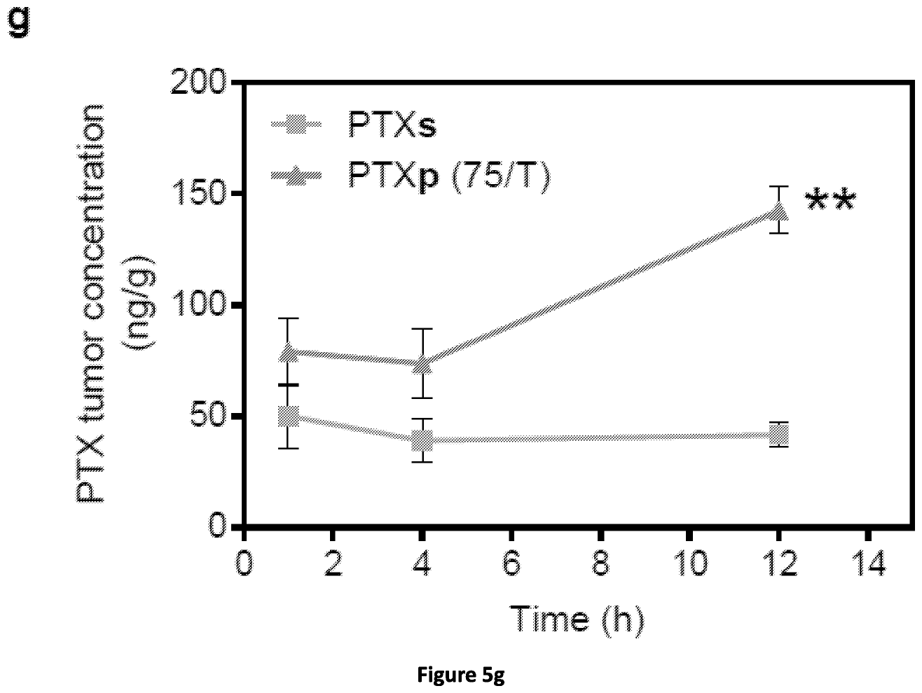

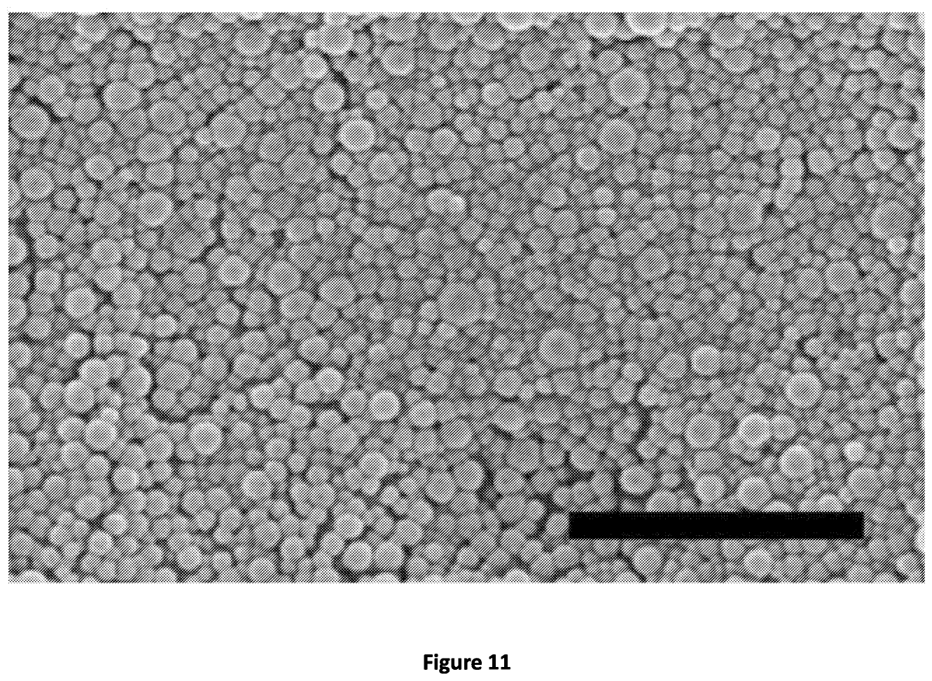

In vivo synthetic lethality and safety of PTXp (75/T) +BIBFp (75/T). After establishing that the combination of BIBFs and PTXp induce synthetic lethality in USC cells with LOF p53, we next expanded to in vivo studies. In order to optimize the therapeutic efficacy of the combinational treatment, we generated PLGA (75/T) NPs loaded with BIBF (denoted as “BIBFp (75/T)”). NP size, zeta potential and encapsulation efficiency are summarized in (Table 1). SEM of the BIBFp (75/T) demonstrated spherical NPs with a smooth surface morphology (FIG. 11). We next confirmed that BIBF administered in NPs does not impact synthetic lethality to PTXp (75/T). The combination therapy of PTXp (75/T) +BIBFp (75/T) promoted a marked decrease in cell viability compared to all other treatments, including PTXp (75/T) +BIBFs (p<0.01, FIG. 5a). Like PTX, BIBF is a reported substrate of P-gp⁶⁵. Thus, loading BIBF in NPs containing TPGS may increase its intracellular accumulation and therapeutic effect.

Studies were extended to an in vivo xenograft model of USC using Hec50co cells. Athymic mice were injected subcutaneously with 2×10⁶ Hec50co cells. Once tumor volumes reached 50 mm³, mice were treated intravenously once per week for 3 weeks with saline (“naive”), PTXs, PTXp (75/T), or the combination therapy of PTXp (75/T) +BIBFp (75/T). PTXp (75/T) alone impeded tumor growth more than PTXs, indicating that delivery of PTX in a NP formulation improve efficacy (FIG. 5b). Treatment with PTXp (75/T) +BIBFp (75/T) was superior to PTXp (75/T) alone, supporting that the combination of BIBF and PTX induces synthetic lethality in vivo. Non-parametric Kruskal-Wallis test demonstrated that only the combination therapy of PTXp (75/T) +BIBFp (75/T) significantly inhibited tumor growth as compared to PTXs (p<0.05) and naive control (p<0.001). Representative images of mice at day 32 are shown in (FIG. 5c).

From a survival perspective, the combination was the only treatment that significantly increased median survival compared to the naive group (FIG. 5d, p<0.05). Specifically, treatment with PTXp (75/T) +BIBFp (75/T) was associated with a median survival of 51 days compared to 43, 41 and 39 days when mice were treated with PTXp (75/T), PTXs or saline, respectively. Thus our combination therapy was able to extend the median survival of the treated mice by 18.6% when compared to those treated with PTXp (75/T) alone. These data are in line with a clinical study of non-small-cell lung cancer (NSCLC) demonstrating that the combination of BIBF and docetaxel (a PTX derivative) extended median survival by 22.3% when compared to docetaxel plus placebo⁴⁰. This effect was only observed in NSCLC patients with adenocarcinoma histology. Since TP53 mutations also predominate in the adenocarcinoma subtype of NSCLC⁶⁶, we speculate that NSCLC patients that responded on this trial may harbor LOF p53, supporting that the concept of synthetic lethality may be applicable to cancers beyond USC.

None of the tested treatments caused significant adverse effects. First, there was no change in animal weight throughout the experiment (FIG. 5e). Second, a full histological analysis using H&E staining showed no signs of necrosis or cell death in the heart, lung, liver, spleen or kidney (FIG. 5f). These data support the in vivo safety of the combination therapy.

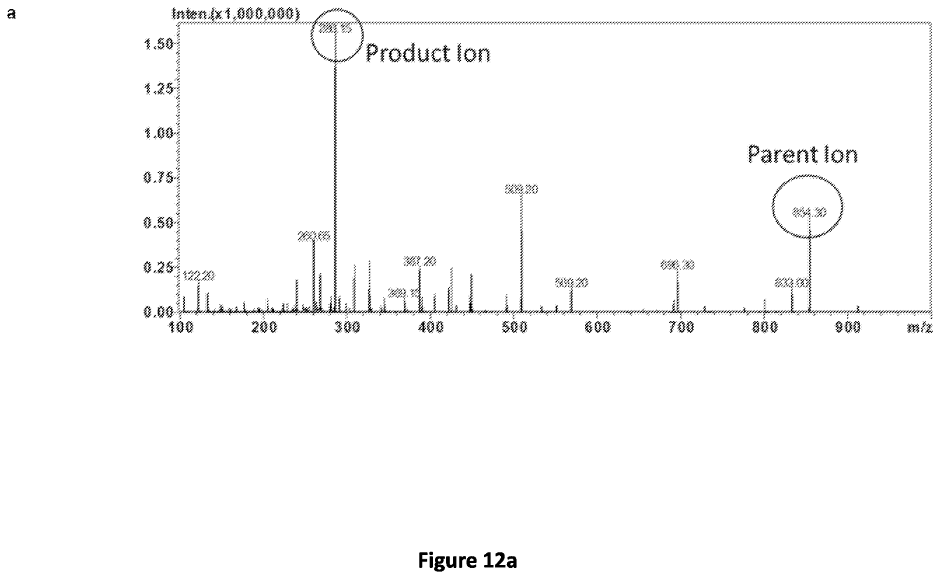

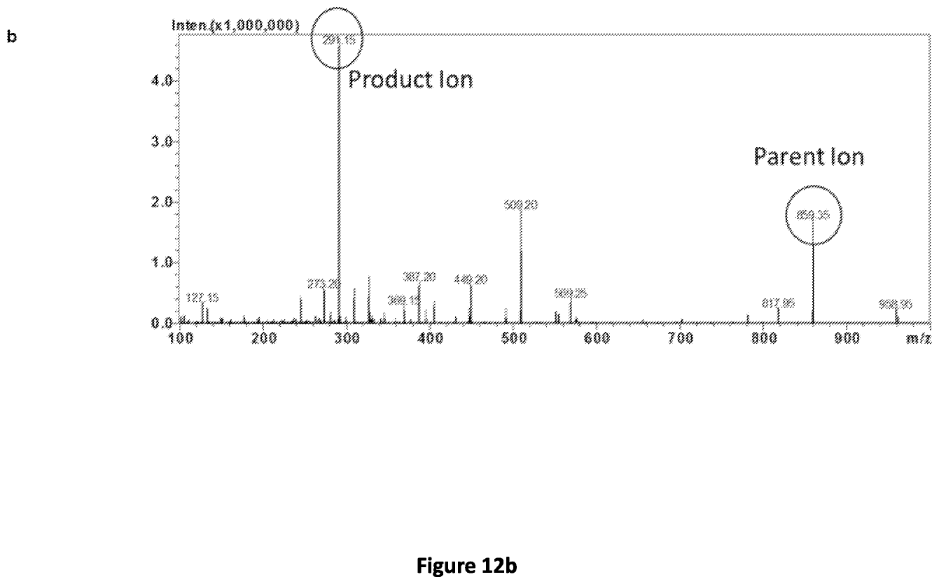

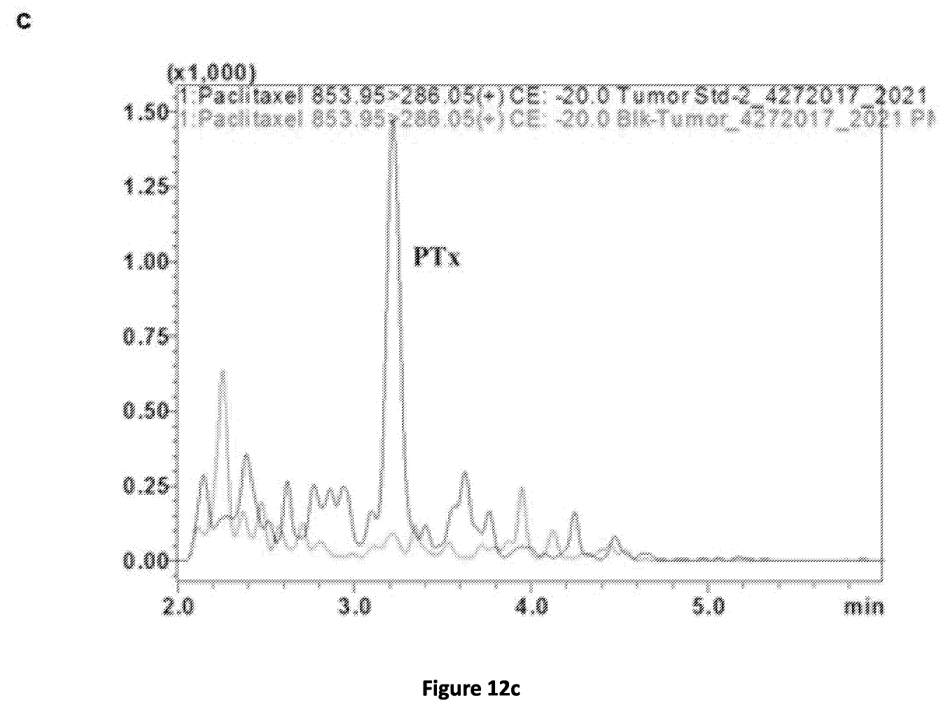

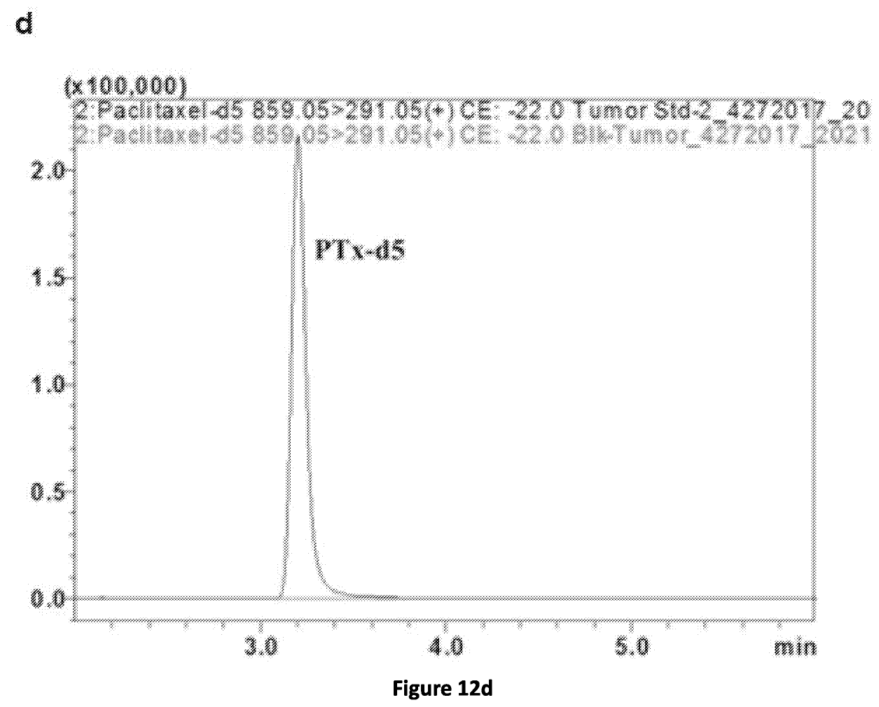

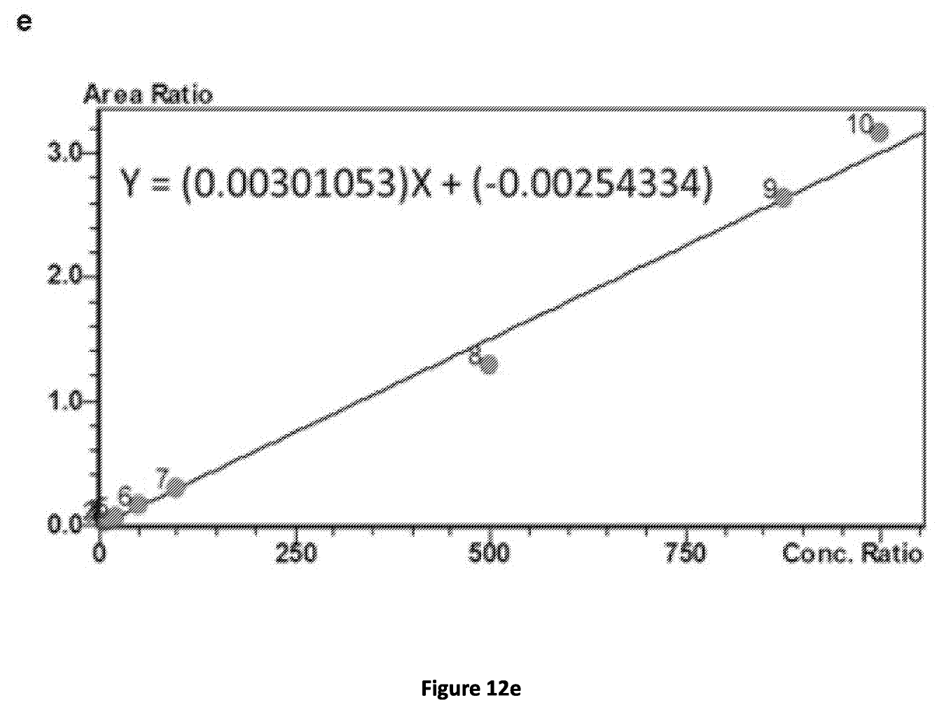

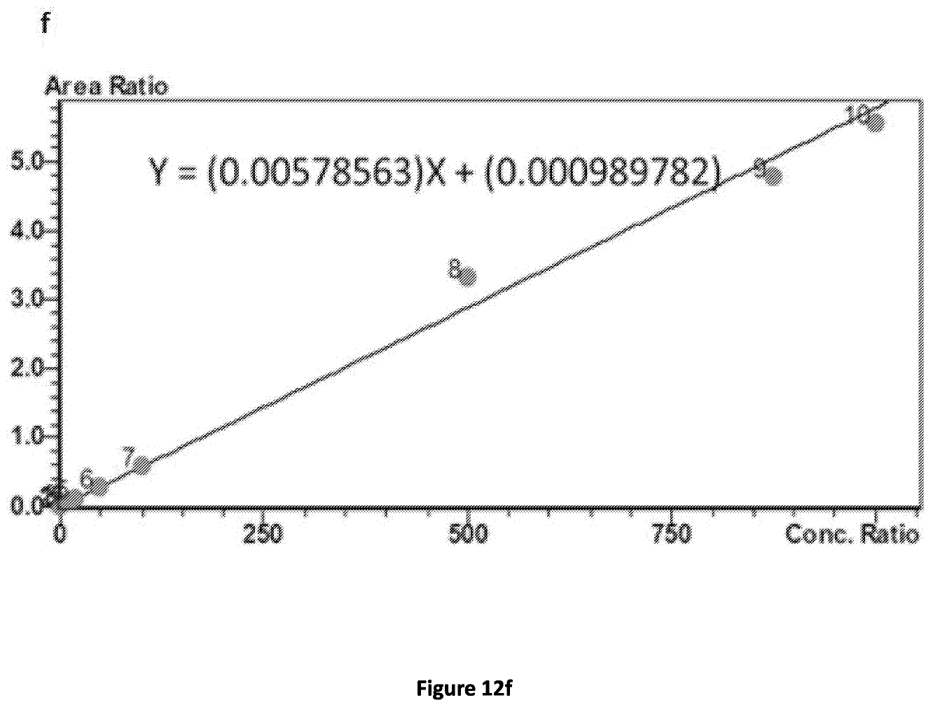

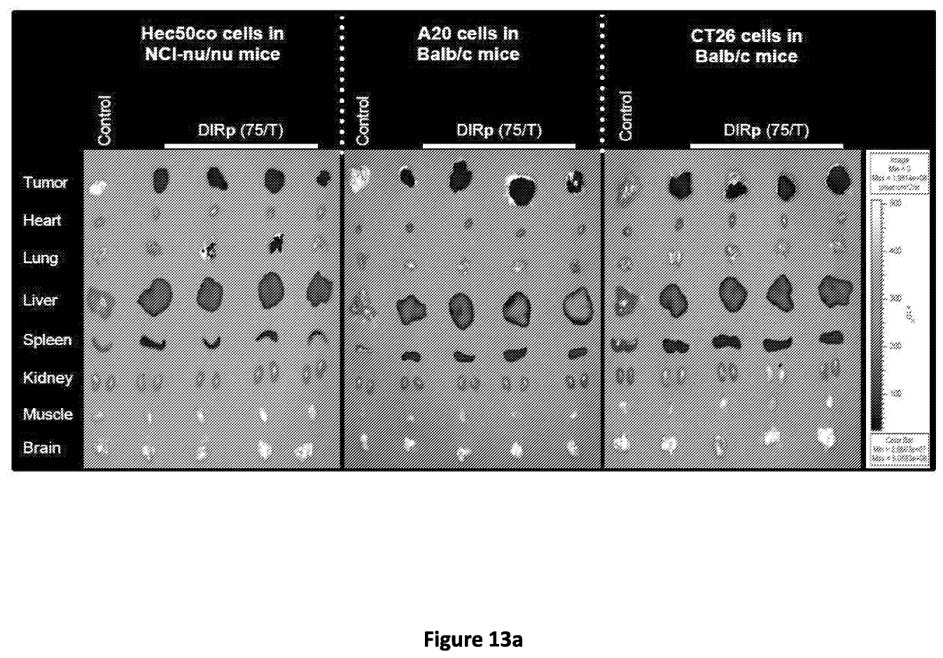

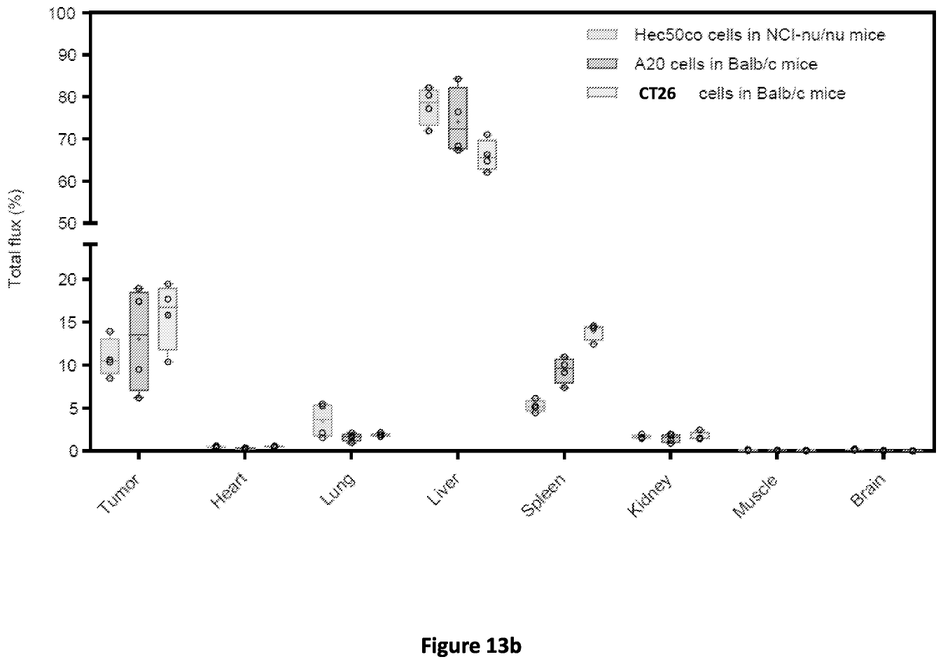

We also analyzed PTX intra-tumoral drug accumulation using a validated LC-MS/MS method (FIG. 12) and found superior accumulation of PTXp (75/T) as compared to PTXs (FIG. 5g). Finally, we examined the biodistribution profile of the near IR fluorescent dye (1,1′-dioctadecyl-3,3,3′,3′-tetramethylindotricarbocyanine iodide, DIR) loaded NPs (DIRp (75/T)) since previous studies have suggested that only a minor fraction of NPs (0.7%) reaches the tumor⁶⁷. In contrast, however, at least 10% of DIRp (75/T) accumulated in tumors (FIG. 13). Together, these data substantiate the potential clinical relevance of our preclinical studies.

Conclusions

The data presented here provide compelling evidence that p53 plays a critical role in response to the combination therapy of PTX and BIBF, such that synthetic lethality only occurs in the setting of LOF p53. Mechanistic studies support that abrogation of the G2/M checkpoint allows cells to prematurely enter M phase, where they undergo cell death through mitotic arrest. Moreover, the specific NP formulation consisting of PLGA at a monomer ratio of 75:25 and TPGS surfactant improves therapeutic efficacy through better drug uptake and accumulation and reduced drug efflux. Together, this conceptual design resulted in enhanced cell killing in vitro and decreased tumor growth in vivo without compromising safety.

In addition to abrogation of the G2/M checkpoint, we considered the possibility that other reported mechanisms of action for BIBF may contribute to the synergy when combined with PTX. BIBF has been shown to inhibit the activity of the drug efflux transporter P-gp⁵⁹. Paclitaxel is a P-gp substrate, which leads to resistance to paclitaxel⁵³. Consistent with this reported mechanism of action for BIBF, we found that, regardless of p53 mutational status, BIBF enhanced accumulation of rhodamine-containing NPs (FIG. 4d&e), and both cell lines exhibited the same magnitude of increase. However, it is important to note that cells with LOF p53 were uniquely sensitive to the combination of BIBF and paclitaxel, supporting that the mechanism of synergy between these two drugs is likely synthetic lethality rather than enhanced drug accumulation.

Another well-established mechanism of action for BIBF is its anti-angiogenic properties³⁸, which is relevant for the in vivo experiments. However, anti-angiogenic activity has been reported after administration of BIBF at a dose of 100 mg/kg orally for five consecutive days³⁸. In our studies, we administered BIBF only once per week, which would likely not be sufficient for an anti-angiogenic effect.

Numerous EC clinical trials have explored the use of many small molecule inhibitors as single agents. To date, only a handful of treatments have improved progression-free survival, with the best results seen with anti-angiogenic agents (bevacizumab⁶⁸, cediranib⁶⁹). However, it should be noted that these treatments only extend tumor-free growth on average by three months, and there is no improvement in overall survival. These data suggest that combinatorial strategies that target specific Achilles' heels in each tumor must be designed in order to improve long-term survival for patients with EC. Data from this study provide the proof-of-concept that synthetic lethality to PTX can be achieved in LOF p53 tumors by the addition of BIBF to the treatment regimen. These findings may extend beyond EC to other cancers types that are typified by TP53 mutations, such as NSCLC and ovarian cancer, where the combination of BIBF with chemotherapy has improved progression-free survival^{40,41}.

### REFERENCES

1. Sorosky, J. I. Endometrial cancer. Obstet Gynecol 120, 383-397, doi:10.1097/AOG.0b013e3182605bf1 (2012).

2. Siegel, R., Naishadham, D. & Jemal, A. Cancer statistics, 2012. CA: A Cancer Journal for Clinicians 62, 10-29, doi:10.3322/caac.20138 (2012).

3. Siegel, R., Naishadham, D. & Jemal, A. Cancer statistics, 2013. CA: A Cancer Journal for Clinicians 63, 11-30, doi:10.3322/caac.21166 (2013).

4. Siegel, R., Ma, J., Zou, Z. & Jemal, A. Cancer statistics, 2014. CA: A Cancer Journal for Clinicians 64, 9-29, doi:10.3322/caac.21208 (2014).

5. Siegel, R. L., Miller, K. D. & Jemal, A. Cancer statistics, 2015. CA: A Cancer Journal for Clinicians 65, 5-29, doi:10.3322/caac.21254 (2015).

6. Siegel, R. L., Miller, K. D. & Jemal, A. Cancer statistics, 2016. CA: A Cancer Journal for Clinicians 66, 7-30, doi:10.3322/caac.21332 (2016).

7. Cancer Facts and FIGS. 2015, <http://www.cancer.org/acs/groups/content/@editorial/documents/document/acspc-044552.pdf>(2015).

8. Banno, K., Yanokura, M., lida, M., Masuda, K. & Aoki, D. Carcinogenic mechanisms of endometrial cancer: involvement of genetics and epigenetics. J Obstet Gynaecol Res 40, 1957-1967, doi:10.1111/jog.12442 (2014).

9. Doll, A. et al. Novel molecular profiles of endometrial cancer-new light through old windows. J Steroid Biochem Mol Biol 108, 221-229, doi:10.1016/j.jsbmb.2007.09.020 (2008).

10. Hamilton, C. A. et al. Uterine papillary serous and clear cell carcinomas predict for poorer survival compared to grade 3 endometrioid corpus cancers. Br J Cancer 94, 642-646, doi:10.1038/sj.bjc.6603012 (2006).

11. Moxley, K. M. & McMeekin, D. S. Endometrial carcinoma: a review of chemotherapy, drug resistance, and the search for new agents. Oncologist 15, 1026-1033, doi:10.1634/theoncologist.2010-0087 (2010).

12. Boruta, D. M., 2nd, Gehrig, P. A., Fader, A. N. & Olawaiye, A. B. Management of women with uterine papillary serous cancer: a Society of Gynecologic Oncology (SGO) review. Gynecol Oncol 115, 142-153, doi:10.1016/j.ygyno.2009.06.011 (2009).

13. Dizon, D. S. et al. A phase II evaluation of nintedanib (BIBF-1120) in the treatment of recurrent or persistent endometrial cancer: An NRG Oncology/Gynecologic Oncology Group Study. Gynecologic oncology 135, 441-445 (2014).

14. Integrated genomic characterization of endometrial carcinoma. Nature 497, 67-73, doi:10.1038/nature12113; http ://www .nature.com/nature/journal/v497/n7447/ab s/nature12113 .html#supplementary-information (2013).

15. Brachova, P., Thiel, K. W. & Leslie, K. K. The consequence of oncomorphic TP53 mutations in ovarian cancer. Int J Mol Sci 14, 19257-19275, doi:10.3390/ijms 140919257 (2013).

16. Agarwal, M. L., Agarwal, A., Taylor, W. R. & Stark, G. R. p53 controls both the G2/M and the G1 cell cycle checkpoints and mediates reversible growth arrest in human fibroblasts. Proceedings of the National Academy of Sciences of the United States of America 92, 8493-8497 (1995).

17. Jordan, M. A. & Wilson, L. Microtubules as a target for anticancer drugs. Nature Reviews Cancer 4, 253-265 (2004).

18. Bartek, J. & Lukas, J. Chk1 and Chk2 kinases in checkpoint control and cancer. Cancer Cell 3, 421-429 (2003).

19. Shiloh, Y. ATM and related protein kinases: safeguarding genome integrity. Nat Rev Cancer 3, 155-168, doi:10.1038/nrc1011 (2003).

20. Kastan, M. B. & Bartek, J. Cell-cycle checkpoints and cancer. Nature 432, 316-323, doi:10.1038/nature03097 (2004).

21. Harper, J. W. & Elledge, S. J. The DNA damage response: ten years after. Mol Cell 28, 739-745, doi:10.1016/j.molce1.2007.11.015 (2007).

22. Jackson, S. P. & Bartek, J. The DNA-damage response in human biology and disease. Nature 461, 1071-1078, doi:10.1038/nature08467 (2009).

23. Reinhardt, H. C. et al. DNA damage activates a spatially distinct late cytoplasmic cell-cycle checkpoint network controlled by MK2-mediated RNA stabilization. Mol Cell 40, 34-49, doi:10.1016/j.molce1.2010.09.018 (2010).

24. Morandell, S. et al. A reversible gene-targeting strategy identifies synthetic lethal interactions between MK2 and p53 in the DNA damage response in vivo. Cell reports 5, 868-877, doi:10.1016/j.celrep.2013.10.025 (2013).

25. O′Neill, T. et al. Determination of substrate motifs for human Chkl and hCdsl/Chk2 by the oriented peptide library approach. J Biol Chem 277, 16102-16115, doi: 10.1074/jbc.M111705200 (2002).

26. Manke, I. A. et al. MAPKAP kinase-2 is a cell cycle checkpoint kinase that regulates the G2/M transition and S phase progression in response to UV irradiation. Mol Cell 17, 37-48, doi:10.1016/j.molce1.2004.11.021 (2005).

27. Boutros, R., Lobjois, V. & Ducommun, B. CDC25 phosphatases in cancer cells: key players? Good targets? Nat Rev Cancer 7, 495-507, doi:10.1038/nrc2169 (2007).

28. Reinhardt, H. C. et al. DNA Damage Activates a Spatially Distinct Late Cytoplasmic Cell-Cycle Checkpoint Network Controlled by MK2-Mediated RNA Stabilization. Molecular Cell 40, 34-49, doi:http://dx.doi.org/10.1016/j.molce1.2010.09.018 (2010).

29. Meng, X. et al. Strategies for Molecularly Enhanced Chemotherapy to Achieve Synthetic Lethality in Endometrial Tumors with Mutant p53. Obstet Gynecol Int 2013, 828165, doi:10.1155/2013/828165 (2013).

30. Meng, X. et al. Induction of mitotic cell death by overriding G2/M checkpoint in endometrial cancer cells with non-functional p53. Gynecol Oncol 128, 461-469, doi:10.1016/j.ygyno.2012.11.004 (2013).

31. Hartwell, L. H., Szankasi, P., Roberts, C. J., Murray, A. W. & Friend, S. H. Integrating genetic approaches into the discovery of anticancer drugs. Science 278, 1064-1068 (1997).

32. Kaelin, W. G. The Concept of Synthetic Lethality in the Context of Anticancer Therapy. Nat Rev Cancer 5, 689-698 (2005).

33. Bryant, H. E. et al. Specific killing of BRCA2-deficient tumours with inhibitors of poly(ADP-ribose) polymerase. Nature 434, 913-917, doi:10.1038/nature03443 (2005).

34. Farmer, H. et al. Targeting the DNA repair defect in BRCA mutant cells as a therapeutic strategy. Nature 434, 917-921, doi:10.1038/nature03445 (2005).

35. Tutt, A. et al. Oral poly(ADP-ribose) polymerase inhibitor olaparib in patients with BRCA1 or BRCA2 mutations and advanced breast cancer: a proof-of-concept trial. Lancet 376, 235-244, doi:10.1016/50140-6736(10)60892-6 (2010).

36. McLornan, D. P., List, A. & Mufti, G. J. Applying synthetic lethality for the selective targeting of cancer. N Engl J Med 371, 1725-1735, doi:10.1056/NEJMra1407390 (2014).

37. Khoo, K. H., Verma, C. S. & Lane, D. P. Drugging the p53 pathway: understanding the route to clinical efficacy. Nat Rev Drug Discov 13, 217-236, doi:10.1038/nrd4236 (2014).

38. Hilberg, F. et al. BIBF 1120: Triple Angiokinase Inhibitor with Sustained Receptor Blockade and Good Antitumor Efficacy. Cancer Research 68, 4774 (2008).

39. Meng, X. et al. Induction of mitotic cell death by overriding G2/M checkpoint in endometrial cancer cells with non-functional p53. Gynecol Oncol 128, 461-469, doi:10.1016/j.ygyno.2012.11.004 (2013).

40. Reck, M. et al. Docetaxel plus nintedanib versus docetaxel plus placebo in patients with previously treated non-small-cell lung cancer (LUME-Lung 1): a phase 3, double-blind, randomised controlled trial. Lancet Oncol 15, 143-155, doi:10.1016/S1470-2045(13)70586-2 (2014).

41. du Bois, A. et al. Standard first-line chemotherapy with or without nintedanib for advanced ovarian cancer (AGO-OVAR 12): a randomised, double-blind, placebo-controlled phase 3 trial. Lancet Oncol 17, 78-89, doi:10.1016/S1470-2045(15)00366-6 (2016).

42. Jain, R. K. & Stylianopoulos, T. Delivering nanomedicine to solid tumors. Nat Rev Clin Oncol 7, 653-664, doi:10.1038/nrclinonc.2010.139 (2010).

43. Acharya, S. & Sahoo, S. K. PLGA nanoparticles containing various anticancer agents and tumour delivery by EPR effect. Adv Drug Deliv Rev 63, 170-183, doi:10.1016/j.addr.2010.10.008 (2011).

44. Albitar, L., Pickett, G., Morgan, M., Davies, S. & Leslie, K. K. Models representing type I and type II human endometrial cancers: Ishikawa H and Hec50co cells. Gynecologic Oncology 106, 52-64, doi:http://dx.doi.org/10.1016/j.ygyno.2007.02.033 (2007).

45. Nishida, M. The Ishikawa cells from birth to the present. Hum Cell 15, 104-117 (2002).

46. Lee, M. J. et al. Sequential application of anticancer drugs enhances cell death by rewiring apoptotic signaling networks. Cell 149, 780-794, doi:10.1016/j.cell.2012.03.031 (2012).

47. Wang, G. et al. Controlled preparation and antitumor efficacy of vitamin E TPGS-functionalized PLGA nanoparticles for delivery of paclitaxel. Int J Pharm 446, 24-33, doi:10.1016/j.ijpharm.2013.02.004 (2013).

48. Mu, L. & Feng, S. S. A novel controlled release formulation for the anticancer drug paclitaxel (Taxol): PLGA nanoparticles containing vitamin E TPGS. J Control Release 86, 33-48 (2003).

49. Collnot, E. M. et al. Vitamin E TPGS P-glycoprotein inhibition mechanism: influence on conformational flexibility, intracellular ATP levels, and role of time and site of access. Mol Pharm 7, 642-651, doi:10.1021/mp900191s (2010).

50. Mu, L. S., P H; Ang, S N; Feng, S S. Study on surfactant coating of polymeric nanoparticles for controlled delivery of anticancer drug. COLLOID AND POLYMER SCIENCE 283, 58-65 (2004).

51. Turk, C. T., Oz, U. C., Serim, T. M. & Hascicek, C. Formulation and optimization of nonionic surfactants emulsified nimesulide-loaded PLGA-based nanoparticles by design of experiments. AAPS PharmSciTech 15, 161-176, doi:10.1208/s12249-013-0048-9 (2014).

52. Duhem, N., Danhier, F. & Preat, V. Vitamin E-based nanomedicines for anti-cancer drug delivery. J Control Release 182, 33-44, doi:10.1016/j.jconre1.2014.03.009 (2014).

53. Szakacs, G., Paterson, J. K., Ludwig, J. A., Booth-Genthe, C. & Gottesman, M. M. Targeting multidrug resistance in cancer. Nat Rev Drug Discov 5, 219-234 (2006).

54. Eytan, G. D., Regev, R., Oren, G., Hurwitz, C. D. & Assaraf, Y. G. Efficiency of P-glycoprotein-mediated exclusion of rhodamine dyes from multidrug-resistant cells is determined by their passive transmembrane movement rate. European journal of biochemistry/FEBS 248, 104-112 (1997).

55. Guarnieri, D., Guaccio, A., Fusco, S. & Netti, P. A. Effect of serum proteins on polystyrene nanoparticle uptake and intracellular trafficking in endothelial cells. Journal of Nanoparticle Research 13, 4295-4309, doi:10.1007/s11051-011-0375-2 (2011).

56. Samadi Moghaddam, M., Heiny, M. & Shastri, V. P. Enhanced cellular uptake of nanoparticles by increasing the hydrophobicity of poly(lactic acid) through copolymerization with cell-membrane-lipid components. Chem Commun (Camb) 51, 14605-14608, doi:10.1039/c5cc06397c (2015).

57. Dinarvand, R., Sepehri, N., Manoochehri, S., Rouhani, H. & Atyabi, F. Polylactide-co-glycolide nanoparticles for controlled delivery of anticancer agents. International Journal of Nanomedicine 6, 877-895, doi:10.2147/IJN.S18905 (2011).

58. Zhu, H. et al. Co-delivery of chemotherapeutic drugs with vitamin E TPGS by porous PLGA nanoparticles for enhanced chemotherapy against multi-drug resistance. Biomaterials 35, 2391-2400, doi:10.1016/j.biomaterials.2013.11.086 (2014).

59. Xiang, Q. F. et al. Effect of BIBF 1120 on reversal of ABCB1-mediated multidrug resistance. Cell Oncol (Dordr) 34, 33-44, doi:10.1007/s13402-010-0003-7 (2011).

60. Reinhardt, H. C., Aslanian, A. S., Lees, J. A. & Yaffe, M. B. p53-Deficient Cells Rely on ATM- and ATR-Mediated Checkpoint Signaling through the p38MAPK/MK2 Pathway for Survival after DNA Damage. Cancer Cell 11, 175-189, doi:http://dx.doi.org/10.1016/j.ccr.2006.11.024 (2007).

61. Rangarajan, S. et al. Novel Mechanisms for the Antifibrotic Action of Nintedanib. Am J Respir Cell Mol Biol 54, 51-59, doi:10.1165/rcmb.2014-04450C (2016).

62. Thottassery, J. V., Zambetti, G. P., Arimori, K., Schuetz, E. G. & Schuetz, J. D. p53-dependent regulation of MDR1 gene expression causes selective resistance to chemotherapeutic agents. Proc Natl Acad Sci U S A 94, 11037-11042 (1997).

63. De Angelis, P. et al. P-glycoprotein is not expressed in a majority of colorectal carcinomas and is not regulated by mutant p53 in vivo. British Journal of Cancer 72, 307-311 (1995).

64. Sampath, J. et al. Mutant p53 cooperates with ETS and selectively up-regulates human MDR1 not MRP1. The Journal of biological chemistry 276, 39359-39367, doi:10.1074/jbc.M103429200 (2001).

65. Nintedanib. Australian Prescriber 39, 62-63, doi:10.18773/austprescr.2016.031 (2016).

66. The Cancer Genome Atlas Research, N. Comprehensive molecular profiling of lung adenocarcinoma. Nature 511, 543-550, doi:10.1038/nature13385: http ://www .nature.com/nature/journal/v511/n7511/abs/nature13385 .html#supplementary-information (2014).

67. Wilhelm, S. et al. Analysis of nanoparticle delivery to tumours. Nature Reviews Materials 1, 16014 (2016).

68. Aghajanian, C. et al. Phase II Trial of Bevacizumab in Recurrent or Persistent Endometrial Cancer: A Gynecologic Oncology Group Study. Journal of Clinical Oncology 29, 2259-2265, doi:doi:10.1200/JCO.2010.32.6397 (2011).

69. Bender, D. et al. A phase II evaluation of cediranib in the treatment of recurrent or persistent endometrial cancer: An NRG Oncology/Gynecologic Oncology Group study. Gynecologic Oncology 138, 507-512, doi:http://dx.doi.org/10.1016/j.ygyno.2015.07.018 (2015).

Supplementary Materials and Methods

Cell culture and mice. LLC-PK1-WT and LLC-PK1-MDR1 cell lines were generously provided by Dr. John Markowitz from the University of Florida. Both cell lines were maintained in DMEM medium containing 10% FBS, 110 mg/mL sodium pyruvate (Gibco) and 1% Pen/Strep. The blood-brain barrier cell line (hCMEC/D3) was purchased from EMD Millipore and maintained according to the manufacturer's protocol. A20 lymphoma and wild type CT26 colon carcinoma cell lines were purchased from ATCC. A20 lymphoma cells were cultured in RPMI-1640 supplemented with 10% FBS, 1 mM sodium pyruvate (Gibco), 10 mM HEPES (Gibco), 50 μM 2-mercaptoethanol (Sigma), and 50 μg/ml gentamicin sulfate (Mediatech, Inc., Va.). CT26 colon cancer cells were cultured in RPMI-1640 supplemented with 10% FBS, 1 mM sodium pyruvate, 10 mM HEPES, and 50 μg/ml gentamicin sulfate. Cell lines were mycoplasma free as determined by MycoAlert mycoplasma detection kit. Female Balb/c mice at the age of 6-8 weeks were purchased from Jackson Laboratories (Bar Harbor, Me.).

Western blot analysis. Western blot analysis was performed as described in section 3.5 of the main manuscript. β-actin (catalogue no. A1978) was purchased from Sigma. Fibroblast Growth Factor Receptor 2 (FGFR2) (catalogue no. 23328) was purchased from Cell Signaling Technology.

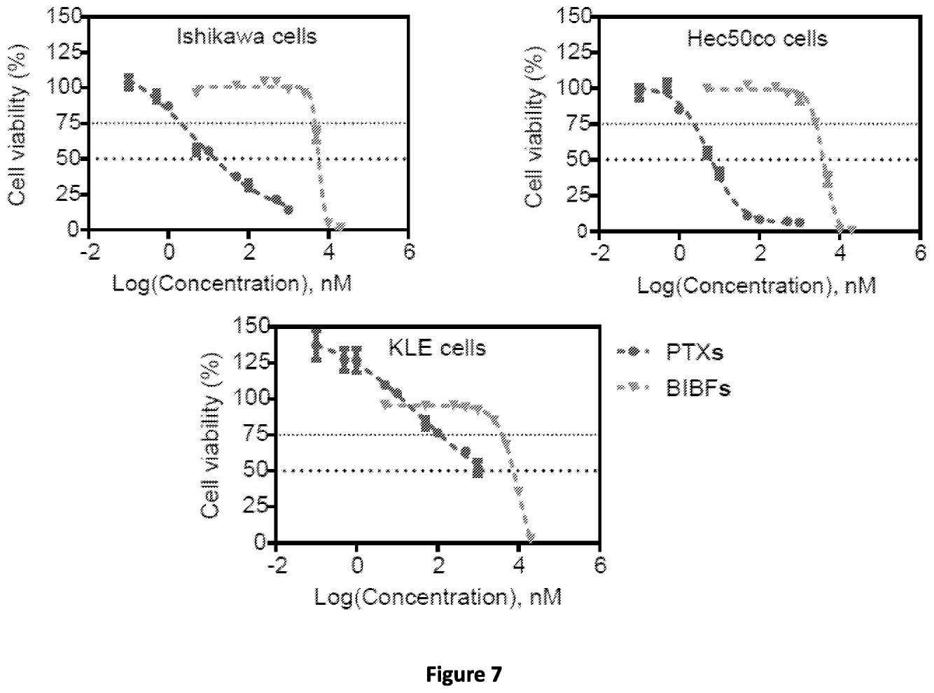

MTS cell proliferation assay. The cell viability analysis was performed as described in section 3.2 of the main manuscript. LLC-PK1-WT and LLC-PK1-MDR1 cells were plated in 96 well plates at a density of 10³ cells per well for 48 h before initiating the treatment. Treatments were added for another 72 h. For FIG. 7, Ishikawa, Hec50co and KLE cells were plated and treated as described in the main manuscript. For FIG. 10, Hec50co cells were plated in 96 well plates at density of 10³ cells per well for 48 h before initiating treatment. Cells were treated with either 5 nM PTXs, 5 nM PTXp (75/T), or Blankp (75/T) =5 nM for 24 h.

Assessment of NP uptake by blood-brain barrier (hCMEC/D3) cells. RHDp (75/T) were prepared using the nanoprecipitation method as described in section 3.3.1 of the main manuscript. Cell uptake was assessed using flow cytometry as mentioned in section 3.3.5 of the main manuscript. Briefly, blood-brain barrier cells (hCMEC/D3) were plated in 12 well plates at a density of 0.5×10⁶ cells per well. After 24 h, RHD (0.01 μg) was added to each well in serum-free medium either in particulate form (RHDp (75/T)) or in soluble form (RHDs). Untreated cells served as the control. Six hours later, the cells were washed with PBS twice, trypsinized and quenched with serum containing media. Cells were then centrifuged at 230 xg for 5 min, resuspended in 300 μL of fresh media and kept on ice until analysis was performed.

Determination of DNA content in Hec50co cells. The DNA content of Hec50co cells was assessed after treatment using CyQUANT® Direct Cell Proliferation Assay Kit (Thermo Fisher Scientific). Briefly, Hec50co cells were seeded into a 96 well plate at a density of 10³ cells per well and incubated for 48 h. After incubation, cells were treated with 5 nM PTXs, 5 nM PTXp (75/T), or Blankp (75/T) =5 nM. Untreated cells served as the control. In order to ensure there were enough viable cells for the assay, the cells were treated for only 24 h. After treatment, the media was removed from the cells and replaced with 100 μL of fresh media and 100 μL of 2× detection reagent. The cells were incubated for 30 min before measuring the fluorescence at λex 480 and λem 535 using a SpectraMax M5 multimode microplate reader. The percent DNA content was calculated as the DNA content of each treatment group normalized to the DNA content of the control cells . The fluorescence intensity of 100 μL of medium plus 100 μL of 2× detection reagent in the absence of cells was used as a blank and subtracted from all data.

Viable cell count using trypan blue in Hec50co cells. Hec50co cells were seeded into 100 mm cell culture dishes at a density of 0.5×10⁶ cells per dish in 9 mL of medium. The cells were incubated for 48 h after which, 3 mL of each treatment was added. The treatment groups were the same as in the DNA content assay. After the cells were treated for 1 day, they were trypsinized and suspended in cell culture medium. The number of viable cells in each sample was determined using trypan blue staining (J.T.Baker Chemical Co., Philipsburg, N.J.).

Determination of ATP content in Hec50co cells. ATP content was determined using the ATP Assay Kit (Abcam, Cambridge, Mass.). The same Hec50co cell samples used for the trypan blue assay were used in the ATP assay following the manufacturer's guidelines. Briefly, the cells were washed with cold PBS, resuspended in 100 μL of ATP buffer and homogenized by pipetting up and down. The insoluble material was pelleted by centrifuging at 13,000×g for 5 minutes and the supernatant was transferred to a new tube. The samples were deproteinized using the Deproteinizing Sample Preparation Kit—TCA (Abcam) according to the manufacturer's protocol. After deproteinization, 50 μL of each sample was added to a 96-well plate along with 50 μL of the reaction mix. A standard curve was constructed alongside the unknown samples according to the provided protocol. After 30 min of incubation at room temperature, the fluorescence intensity was measured at kex 535 and kem 587 using SpectraMax M5 multimode microplate reader. The ATP content of unknown samples was determined using the standard curve and linear regression. Finally, the samples were normalized to the number of cells determined during the trypan blue assay.

Apoptosis assay with annexin V/propidium iodide staining in Hec50co cells. Cellular apoptosis of Hec50co cells was determined using the eBioscienceTM Annexin V Apoptosis Detection Kit (Thermo Fisher Scientific). For this assay, the cells were seeded in 6-well plates at a density of 3×10⁴ cells per well in 4.5 mL of medium and incubated for 48 h. Afterwards, 1.5 mL of each treatment was added to the wells (the same treatment groups as in the DNA, ATP and trypan blue assays). After 24 h of treatment, the cells were rinsed once with PBS and once with the 1× binding buffer provided in the kit. Then, the cells were resuspended in 1× binding buffer at a concentration of 1-5×10⁶ cells/mL. Next, 5 μL of FITC-Annexin V was added to 100 μL of the cell suspension. The samples were incubated for 15 min at room temperature. After incubation, the cells were washed with 1× binding buffer and resuspended in 200 μL of 1× binding buffer. Five μL of propidium iodide (PI) staining solution was added to the cell suspensions. The samples were analyzed using flow cytometry. Data were gated as indicated in FIG. 10 to determine the percentage of cells in apoptosis.

Bright field microscopic evaluation of Hec50co cells. Hec50co cells were grown in 6-well plates at a density of 3×0⁴ cells per well in 4.5 mL of medium and incubated for 48 h. After incubation, 1.5 mL of each treatment was added to the wells (the same treatment groups as in the DNA, ATP, trypan blue and apoptosis assays). After 1 day, the cells were analyzed with 10× magnification using an Olympus inverted microscope (CKX41, Center Valley, Pa.). Images were acquired with an Olympus DP70 digital camera.

Quantitative Estimation of PTX in Murine Tumors Using LC-MS/MS

LC-MS/MS condition for PTX. A Shimadzu LC-MS/MS system (LC-MS/MS 8060, Shimadzu, Japan), LC system equipped with two pumps (LC-30 AD) and column oven (CTO-30AS) along with an auto-sampler (SIL-30AC) was used to inject 10 μL aliquots of the processed samples.

Mass spectrometric detection was performed on an 8060 mass spectrometer equipped with a DUIS source in positive mode. The MS/MS system was operated at unit resolution in the multiple reaction monitoring (MRM) mode, using precursor ion→product ion combinations of 854.30→286.15 m/z for PTX and 859.35→291.15 m/z for the internal standard (IS) (PTX-d5, Toronto Research Chemicals Inc., Toronto, ON, Canada). The compound-dependent mass spectrometer parameters, such as temperature, voltage, gas, and pressure, were optimized by auto method optimization via precursor ion search for each analyte and the internal standard (IS) using a 0.5 μg/mL solution in acetonitrile. PTX and PTX-d5 were detected in the positive ionization mode with the following instrument dependent mass spectrometer parameters: nebulizer gas: 2.0 L/min; heating gas: 10 L/min; drying gas: 10 L/min; interface temperature: 375° C.; desolvation line temperature: 250° C.; heat block temperature: 400° C. and interface. UPLC and MS systems were controlled by LabSolutions LCMS Ver.5.6. (Shimadzu Scientific, Inc).

The compound PTX resolution and acceptable peak shape were achieved on a ACE Excel C18 (1.7 μm, 2.1×100 mm, Advance Chromatography Technologies, LTD., UK) column protected with a C18 guard column (Phenomenex, Torrance CA). The PTX-d5 was used as the IS. The mobile phase consisted of 0.2% formic acid in water (mobile phase A) and acetonitrile (ACN) (mobile phase B), at a total flow rate of 0.25 mL/min. The chromatographic separation was achieved using 7 min gradient elution. The initial mobile phase composition was 50% B, gradually increased to 95% B over 5 min, then held constant at 95% B for 1.0 min, and finally brought back to initial condition of 500% B in 0.5 min followed by 1-min re-equilibration. The injection volume of all samples was 10 μL.

Stock, standard and quality control samples preparation. Stock solutions (1 mg/mL) of PTX, and PTX-d5 (IS) were made in acetonitrile. The calibration standard stocks of analytes were prepared by step-wise dilution of the stock solution in acetonitrile over the concentration range of 0.5-1000 ng/mL. Quality control samples (QCs) at four different concentrations were used: lower limit of quantification (LLOQ—0.5 ng/mL), low quality control (LQC—2 ng/mL), middle quality control (MQC—200 ng/mL) and high quality control (HQC—750 ng/mL). QCs were prepared separately in three replicates, independent of the calibration standards. The IS was diluted to 1000 ng/mL in acetonitrile for spiking into tumor samples.

Sample preparation. The plasma and tumor samples were processed using a solid phase extraction technique (SPE). The samples were prepared by spiking 10 μL of appropriate calibration stock into 200 μL of blank tumor homogenate, and 10 μL of the IS solution (1000 ng/mL) was added. Tumor was homogenized in water (1:4) and tumor samples were centrifuged for 5 min at 3500 rpm prior to loading to the SPE cartridge. The SPE was carried out using Agilent bond Elute C18, 50 mg 1 mL Cartridge (Agilent). Cartridges were conditioned with 1 mL acetonitrile and followed by 1 mL water. Tumor samples (200 μL) spiked with 10 μL spiking standard and 10 μL IS, were diluted to 0.8 mL with 0.1% formic acid (FA) and then loaded into the SPE cartridges. The cartridges were washed with 1 mL of aqueous 5% acetonitrile and 0.5% formic acid. Analytes were eluted with 2 mL of acetonitrile. The eluents were collected in glass tubes and evaporated to dryness under nitrogen in water bath set at 50° C. The dry residues were finally reconstituted in 100 μL 0.1% formic acid: acetonitrile (50:50) and 10 μL supernatant injected onto the HPLC.

Method Validation. The developed LC-MS/MS method was validated as per US-FDA guidance with respect to selectivity, specificity, lower limit of quantification (LLOQ), accuracy, precision, and matrix effect¹.

The sensitivity of the method was determined from the signal-to-noise ratio (S/N) of the response of analyte in calibration standards. The S/N ratio should be greater than three for the limit of detection (LOD) and greater than 10 for the LLOQ. The calibration curves were established by plotting the peak area ratio (analyte/IS) versus concentration for all analytes.

Intra- and inter-day accuracy and precision were evaluated from replicate PTX (n=5) of QC samples containing analytes at different concentrations (LLOQ, LQC, MQC and HQC) prepared on the same day. The precision was calculated in terms of % relative standard deviation (% R.S.D.). The accuracy was expressed as % Bias. The criteria for acceptability of the data included accuracy within ±15% standard deviation (S.D.) from the nominal values and a precision within ±15% R.S.D. except for LLOQ, where it should not exceed ±20% of accuracy as well as precision.

% Bias=(observed concentration−nominal concentration)/nominal concentration×100

The carry-over was checked by injecting two zero samples directly after injecting an HQC sample. The response of the first zero sample should be <20% of the response of a processed LLOQ sample.

The dilution effect was investigated to ensure that tumor homogenate samples could be diluted with water without affecting the result. Analytes spiked stripped serum prepared at 2000 ng/mL concentrations were diluted with stripped serum at dilution factors of 5 and 10 in five replicates and analyzed. As part of the validation, five replicates had to comply with both precision of ≤15% and accuracy of 100±15% similar to other QCs samples.

The absolute recovery of PTX and IS were calculated by comparing the peak area of QC samples (LQC, MQC and HQC, n=3) in plasma with corresponding standard concentrations prepared in reconstitution solvent. The recovery was deemed acceptable if the % coefficient of variation (CV) was ±20% among the mean recoveries at LQC and HQC levels.

Preparation of DiR-loaded PLGA NPs. The near infrared (IR) fluorescent DIR dye (1,1′-dioctadec yl-3,3,3′,3′-tetramethylindotricarbocyanine iodide, Invitrogen) was loaded in PLGA NPs (DIRp (75/T)) to aid in tracking the biodistribution of these NPs once administered intravenously. DIRp (75/T) were prepared by the nanoprecipitation method as described in section 3.3.1 of the main manuscript using 1 mg of DIR dye. The loading of DIR was determined by dissolving a known amount NPs in DMSO. A standard curve was constructed from known concentrations of DIR dissolved in DMSO. Fluorescence detection was used to quantify the amount of DIR in the NP suspension at kex 750 and kem 780 using a SpectraMax M5 multimode microplate reader.

Biodistribution study. NCI-nu/nu mice were challenged with 2×10⁶ Hec50co cells. Balb/c mice were challenged with either 3×10⁶ CT26 cells or 5×10⁶ A20 cells. Once the tumor size reached ˜500 mm³, the mice were IV injected (retro-orbital injections) with equal doses of DIRp (75/T) equivalent to 5 μg DIR. Forty-eight hours after the injection of NPs, the organs of the mice were harvested and analyzed using an IVIS-200 instrument (Xenogen, PerkinElmer, Waltham, Mass.) with an ICG filter. Images were analyzed using Living Image software by measuring the total flux from each organ. The baseline flux for each organ was determined from the control sample and was subtracted from all data. To determine percent total flux, the individual flux measurements from each organ of the mouse was summed. Then, the flux contribution from each organ was divided by the total flux from the summation of all organs and multiplied by 100 (see the equation below).

$$Total Flux ( \% ) = \frac{Flux of individual organ}{Total flux of all organs summed together} \times 100$$

Supplementary Results and Discussion for the LC-MS/MS Quantification of PTX

Chromatographic and mass spectrometric conditions optimization. To obtain the selectivity and sensitivity for all analytes, several chromatographic and mass spectrometric conditions were optimized. The selection of the ionization mode was based on the comparison of obtained sensitivity with electro spray ionization (ESI) and atmospheric pressure chemical ionization (APCI) source. The results showed that ESI in positive mode could offer much higher intensity for the analytes than APCI (data not shown). The fragmentation of PTX and IS were auto optimized via precursor ion search of approximately 1000 ng/mL of stock solution of each analyte. The most abundant precursor>product ions in terms of better sensitivity for PTX and PTX-d5 at m/z 854.30→286.15 and 859.35→291.15 (FIG. 12a&b). These ions represented the fragmentation at the ester bond and a loss of the taxane structure. The compound dependent parameters such as voltage potential Q1 −26 and −28 (V) and Q3 −20 and −30 (V), collision energy (CE) −20 and −22, were also optimized to obtain the highest signal intensity for PTX and IS, respectively.

Chromatographic conditions, especially the composition of mobile phase and different analytical columns were optimized to achieve good resolution and symmetrical peak shapes of the analytes, as well as a short run time. The suitability and robustness of the method were evaluated using different varieties of reverse phase HPLC columns ranging from 50 to 150 mm in length (data not shown). Complete and rapid chromatographic resolution of analytes and IS was achieved on ACE Excel C18 column (1.7 μm, 100×2.1 mm) equipped with a C18 guard column. A better chromatogram with symmetrical peak shape was obtained using 0.2% FA and acetonitrile at a flow rate of 0.25 mL/min. with 40° C. as the column temperature. The representative overlay chromatograms with blank tumor homogenate in FIG. 12c&d show no interference of endogenous compounds at the retention time of PTX (3.2 min) and PTX-d5 (3.2 min) for samples spiked at 1.0 ng/mL concentration. The PTX-d5 was selected as the IS for PTX in this method. They had similar chromatographic behaviors and similar ionization responses in ESI mass spectrometry to that of analytes.

Method Validation. The method was validated for PTX using three calibration curves prepared on three days. The calibration curves were established by plotting the peak area ratio (peak area analyte/peak area IS) versus nominal concentration least-squares linear regression analysis with a weighting factor of 1/x². The calibration curves were linear over the concentration range of 0.5-1000 ng/mL with a correlation coefficient r²≥0.9980±0.0023 (FIG. 12e&f). The intra-day inter-day accuracy and precision at five replicates of four different QCs (LLQC-QC, LQC, MQC and HQC) was found within acceptable 85-115% limits. A processed zero blank sample (Blank+IS) injected after ULOQ samples showed peak area <5% of LLOQ resulting in no carry over effect.

The precision for dilution integrity of 1:5 and 1:10 dilution were within acceptable limit for PTX, which is within the acceptance limits of ±15% for precision (CV) and 85.0-115.0% for accuracy. The results suggested that plasma or tumor samples whose concentrations above upper limit of quantitation can be determined by appropriate dilution.

The % mean recovery was determined by measuring the response of the extracted plasma quality control samples at HQC, MQC and LQC against un-extracted quality control samples at HQC, MQC and LQC. The mean recovery of all three QC levels was 95.60%, whereas the mean recovery of IS was 90.71%.

Supplementary References

1. Food and Drug Administration Centre for Drug Evaluation and Research (FDA). Guidance for Industry-Bioanalytical Method Validation. Silver Spring, Md: Center for Drug Evaluation and Research, US Department for Health and Human Services, May 2001, 2013.

2. Konecny, G. E. et al. Activity of the fibroblast growth factor receptor inhibitors dovitinib (TKI258) and NVP-BGJ398 in human endometrial cancer cells. Mol Cancer Ther 12, 632-642, doi:10.1158/1535-7163.MCT-12-0999 (2013).

3. Winterhoff, B. & Konecny, G. E. Targeting fibroblast growth factor pathways in endometrial cancer. Curr Probl Cancer 41, 37-47, doi:10.1016/j.currproblcancer.2016.11.002 (2017).

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

1. A pharmaceutical composition comprising as components:
(a) a cytoskeletal drug that blocks progression of cells through mitosis;
(b) an anti-angiogenic drug;
(c) nanoparticles, wherein the nanoparticles comprise the cytoskeletal drug, the anti-angiogenic drug, or both of the cytoskeletal drug and the anti-angiogenic drug either in separate nanoparticles or mixed in the same nanoparticles;
(d) optionally a surfactant; and
(e) optionally liposomes and/or components of liposomes.

2. The composition of claim 1, wherein the cytoskeletal drug is paclitaxel (PTX) or a derivative thereof, or wherein the anti-angiogenic drug is a tyrosine kinase inhibitor that inhibits a receptor selected from the group consisting of vascular endothelial growth factor receptor (VEGFR), fibroblast growth factor receptor (FGFR), platelet-derived growth factor receptor (PDGFR), or any combination thereof.

3. (canceled)

4. The composition of claim 1, wherein the anti-angiogenic drug is BIBF-1120.

5. The composition of claim 1, wherein the nanoparticles comprise the cytoskeletal drug at a concentration of at least about 5, 10, 20, 30, 40, 50, 100, or 200 μg/mg nanoparticle or within a concentration range bounded by any of these values, or wherein the nanoparticles comprise the anti-angiogenic drug at a concentration of at least about 5, 10, 20, 30, 40, 50, 100, or 200 μg/mg nanoparticle or within a concentration range bounded by any of these values.

6. (canceled)

7. (canceled)

8. The composition of claim 1, wherein the nanoparticles have an average effective diameter of <500 nm, and preferably have an average effective diameter of <400, 300, 200, 150, 100, or 50 nm, or have an average effective diameter within a range bounded by any of these values.

9. The composition of claim 1, wherein the nanoparticles are biodegradable nanoparticles that comprise a biodegradable polymer.

10. The composition of claim 9, wherein the biodegradable polymer of the biodegradable nanoparticles comprises polymerized carbohydrate monomers.

11. The composition of claim 9, wherein the biodegradable nanoparticles comprise poly(lactic-co-glycolic acid) (PLGA).

12. The composition of claim 11, wherein the wherein the biodegradable nanoparticles comprise PLGA 75:25 or PLGA 50:50.

13. The composition of claim 1, wherein the composition comprises a surfactant and the surfactant comprises a water soluble polymer coupled to a hydrophobic molecule.

14. The composition of claim 1, wherein the composition comprises a surfactant and the surfactant is polyethylene glycol coupled to a tocopherol, preferably D-a-tocopherol glycol 1000 succinate (i.e., TPGS).

15. The composition of claim 1, wherein one or more of the components of the pharmaceutical composition inhibits the P-glycoprotein (P-gp) efflux transporter.

16. (canceled)

17. The composition of claim 1, wherein the composition comprises a surfactant (e.g., TPGS) and the surfactant inhibits the P-glycoprotein (P-gp) efflux transporter.

18. The composition of claim 1, wherein the composition further comprises a T-cell stimulatory agent.

19. The composition of claim 1, wherein the composition further comprises an immune checkpoint inhibitor.

20. The composition of claim 1, comprising:
(a) PTX;
(b) BIBF-1120;
(c) nanoparticles, wherein the nanoparticles comprise PTX, BIBF-1120, or both of PTX and BIBF-1120 either in separate nanoparticles or mixed in the same nanoparticles; and
(d) TPGS.

21. A method for treating a subject having a cancer characterized by loss-of-function of the p53 protein, the method comprising administering to the subject the pharmaceutical composition of claim 1.

22. (canceled)

23. (canceled)

24. A method for treating a subject having a cancer characterized by loss-of-function of the p53 protein, the method comprising:
(a) administering to the subject a cytoskeletal drug that blocks progression of the cancer cells through mitosis, preferably PTX; and
(b) administering to the subject an anti-angiogenic drug, preferably BIBF-1120.

25.-27. (canceled)

28. A method for treating a subject having a cancer susceptible to synthetic lethality, the method comprising administering to the subject a composition comprising nanoparticles and one or more cytotoxic and/or chemotherapeutic drugs that induce synthetic lethality.

29.-33. (canceled)

34. A method for treating a subject having a cancer characterized by p53 deficiency or downregulation, the method comprising administering to the subject a pharmaceutical composition comprising nanoparticles, a cytoskeletal drug that block progression of cancers cells through mitosis, and an inhibitor of the p38 MAPK pathway, wherein less than about 100 mg of the inhibitor of the p38 MAPK pathway is administered to the subject.

35. (canceled)
